(19) United States
(12) Patent Application Publication
(10) Pub. No.: US 2005/0197336 A1
(43) Pub. Date: Sep. 8, 2005

(54) Inhibitors of histone deacetylase

(75) Inventors: Sampath K. Anandan, Fremont, CA (US); Xiao-Yi Xiao, San Diego, CA (US); Dinesh V. Patel, Fremont, CA (US); John S. Ward, Reedwood City, CA (US)

Correspondence Address: FOLEY & LARDNER LLP, 1530 PAGE MILL ROAD, PALO ALTO, CA, 94304, US

(73) Assignee: Miikana Therapeutics Corporation

(21) Appl. No.: 10/992,303

(22) Filed: Nov. 17, 2004

Related U.S. Application Data
(60) Provisional application No. 60/551,644, filed on Mar. 8, 2004.

(51) Int. Cl. A61K 31/5377; A61K 31/496; C07D 417/14; C07D 417/04

(52) U.S. Cl. 514/235.5; 544/120; 544/369; 514/254.02

(57) ABSTRACT

Disclosed are compounds which inhibit histone deacetylase (HDAC) enzymatic activity. Also disclosed are pharmaceutical compositions comprising such compounds as well as methods to treat conditions, particularly proliferative conditions, mediated at least in part by HDAC.

### CROSS-REFERENCE TO RELATED CASES

This application claims the benefit under 35 U.S.C. §119 (e) of U.S. Provisional Application Ser. No. 60/551,644 filed Mar. 8, 2004 which application is incorporated herein by reference in its entirety.

### BACKGROUND OF THE INVENTION

Field of the Invention

This invention relates to compounds which inhibit histone deacetylase (HDAC) enzymatic activity. This invention is also directed to pharmaceutical compositions comprising such compounds as well as to their use to treat conditions, particularly proliferative conditions, mediated at least in part by HDAC.

### REFERENCES

The following publications are cited in this application as superscript numbers:
¹ Marks, et al., Nature Reviews: Cancer 1: 194-202 (2001)
² Finnin, et al., Nature, 401:188-193 (1999)
³ Geerts, et al., European Patent Application Publication No. 0 827 742, published Mar. 11, 1998

All of the above publications are incorporated herein by reference in their entirety to the same extent as if each individual publication, patent or patent application was specifically and individually indicated to be incorporated by reference in its entirety.

### STATE OF THE ART

In all eukaryotic cells, genomic DNA in chromatine associates with histones to form nucleosomes. Each nucleosome consists of a protein octamer made up of two copies of each histone: H2A, H₂B, H3 and H4. DNA winds around this protein core, with the basic amino acids of the histones interacting with the negatively charged phosphate groups of the DNA. The most common posttranslational modification of these core histones is the reversible acetylation of the ε-amino groups of conserved highly basic N-terminal lysine residues. The steady state of histone acetylation is established by the dynamic equilibrium between competing histone acetyltransferase(s) and histone deacetylase(s) herein referred to as HDAC. Histone acetylation and deacetylation has long been linked to transcriptional control. The recent cloning of the genes encoding different histone acetyltransferases and histone deacetylases provides a possible explanation for the relationship between histone acetylation and transcriptional control. The reversible acetylation of histones can result in chromatin remodeling and as such act as a control mechanism for gene transcription. In general, hyperacetylation of histones facilitates gene expression, whereas histone deacetylation is correlated with transcriptional repression. Histone acetyltransferases were shown to act as transcriptional coactivators, whereas deacetylases were found to belong to transcriptional repression pathways.

The dynamic equilibrium between histone acetylation and deacetylation is essential for normal cell growth. Inhibition of histone deacetylation results in cell cycle arrest, cellular differentiation, apoptosis and reversal of the trans-formed phenotype. Therefore, HDAC inhibitors can have great therapeutic potential in the treatment of cell proliferative diseases or conditions.¹

The study of inhibitors of histone deacetylases (HDAC) indicates that indeed these enzymes play an important role in cell proliferation and differentation. The inhibitor Trichostatin A (TSA) causes cell cycle arrest at both the G1 and G2 phases, reverts the transformed phenotype of different cell lines, and induces differentiation of Friend leukemia cells and others. TSA (and subroylanilide hydroxamic acid SAHA) have been reported to inhibit cell growth, induce terminal differentiation, and prevent formation of tumors in mice.²

Trichostatin A has also been reported to be useful in the treatment of fibrosis, e.g., liver fibrosis and liver chirrhosis.³

In view of the above, there is an ongoing need for inhibitors/antagonists of HDAC.

### SUMMARY OF THE INVENTION

This invention provides compounds that inhibit HDAC activity and, accordingly, are useful as anti-proliferative agents in the treatment of proliferative diseases.

Accordingly, in one of its composition aspects, this invention is directed to a compound of formula I: 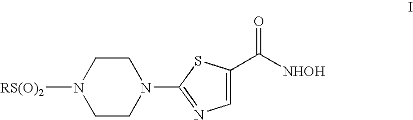
wherein:
R is selected from the group consisting of alkyl, substituted alkyl, alkenyl, substituted alkenyl, amino, substituted amino, aryl, substituted aryl, cycloalkyl, substituted cycloalkyl, heteroaryl, substituted heteroaryl, heterocyclic and substituted heterocyclic;
and tautomers, isomers, prodrugs and pharmaceutically acceptable salts thereof
with the proviso that R is not 4-aminophenyl.

In one embodiment, R is preferably aryl and more preferably is phenyl or naphthyl (e.g., 2-napthyl).

In another embodiment, R is preferably substituted aryl and more preferably, R is a substituted phenyl group selected from the group consisting of:
3-methoxyphenyl, 4-methoxyphenyl, 3,4-dimethoxyphenyl, 4-trifluoro-methoxyphenyl, 2-trifluroromethylphenyl, 3-trifluroromethylphenyl, 4-tri-fluoromethylphenyl, 4-nitrophenyl, 4-acetylphenyl, 4-[(N-morpholino)-methyl]phenyl, 4-[(N-pyrrolidinyl)methyl]phenyl, 4-(N,N-dimethylamino-methyl)phenyl, 5-(N,N-dimethylamino)naphthyl, 4-pyrrolind-1-ylphenyl, 4-acetamidophenyl, 4-methyl-2,3-dihydrobenzisoxazinyl, 2,3-dihydro-benzofuran-5-yl, 2,1,3-benzothiadiazol-5-yl, 2,3-dihydro-1,4-benzodioxin-6-yl, 2-chlorophenyl, 2-chloro-6-methylphenyl, 3-chloro-2,5-dimethylphenyl, 3-chlorophenyl, 3,4-dichlorophenyl, 3-chloro-2-methylphenyl, 3-chloro-4-methylphenyl, 4-chlorophenyl, 3-cyanophenyl, 2-fluorophenyl, 3-fluorophenyl, 3,4-difluorophenyl, 3,5-difluorophenyl, 4-fluorophenyl, 4-fluoro-2-methylphenyl, 3-fluoro-4-methylphenyl, 5-fluoro-2-methylphenyl, 4-methyl-sulfonylphenyl, 2-methylphenyl, 3-methylphenyl, 3-hydroxymethylphenyl, 3-(N,N-dimethylaminomethyl)phenyl, 3-(pyrrolidin-1-yl)methylphenyl, 4-ethylphenyl, 4-methylphenyl, 3,4-dimethylphenyl, 3,5-dimethylphenyl, 4-iso-propylphenyl, 4-n-propylphenyl, t-butylphenyl, (4-pyrazol-1-yl)phenyl, 3-biphenyl, 4-biphenyl, 4-(3-N,N-dimethylaminomethylphenyl)phenyl, 4-(3-N,N-dimethylaminophenyl)phenyl, 4-[(3-pyrrolind-N-ylmethyl) phenyl]phenyl, 4-[(N-morpholinocarbonyl)phenyl]phenyl, 4-(N,N-dimethylaminocarbonylphenyl)phenyl, 4-(4-fluorophenyl)phenyl, 4-(pyrid-4-yl)phenyl, 4-(3-chlorophenyl)phenyl, 4-(2-chlorophenyl)phenyl, 4-(3-fluorophenyl)-phenyl, 4-(2-furanyl)phenyl, 4-[2-(pyrrrolidin-N-ylmethyl)thien-3-yl]phenyl, 4-[5-(pyrrrolidin-N-ylmethyl)thien-2-yl]phenyl, 4-[3-(pyrrrolidin-N-ylmethyl)thien-2-yl]phenyl, 4-[3-(4-methylpiperidin-1-yl)phenyl]phenyl, 4-[(3-(N-methyl-N-{2-N,N-dimethyleth-1-yl}aminomethyl)phenyl]phenyl, 4-[(3-(N-methyl-N-ethylaminomethyl)phenyl]phenyl, 4-[(3-(N-methyl-N-isopropyl}aminomethyl)phenyl]phenyl, 4-(methylsulfonamidophenyl)phenyl, 4-[1,3-(benzodioxol-5-yl)]phenyl, 4-(pyrimidin-5-yl)phenyl, 2-(2-methyl-thiopyrimidin-4-yl)thien-5-yl, 4-[4-(acetamidophenyl)]phenyl, 4-(2-N,N-dimethylaminothien-3-yl)phenyl, and 4-(pyrid-3-yl)phenyl.

In another embodiment, R is preferably heteroaryl. Preferred heteroaryls include, by way of example, thien-2yl, pyrid-2-yl, pyrid-3-yl, and benzothiofuran-2-yl.

In another embodiment, R is preferably substituted heteroaryl. Preferred substituted heteroaryls include, by way of example, 3,5-di-methylisoxazol-4-yl, 2-(4-morpholino)pyrid-5-yl, and 2-phenoxypyrid-5-yl.

In another embodiment, R is preferably alkyl or substituted alkyl. Preferred alkyl and substituted alkyl include, by way of example, n-butyl, benzyl, and 2-phenylethyl.

In yet another embodiment, R is preferably alkenyl or substituted alkenyl. Preferred alkenyl and substituted alkenyl include, by way of example, trans-2-phenylethen-1-yl.

Still further, R is preferably amino or substituted amino such as dimethylamino.

Even still further, R is a substituted heterocyclic group such as 1-methyl-imidazol-4-yl.

One preferred aspect of this invention is directed to compounds of formula II as follows: 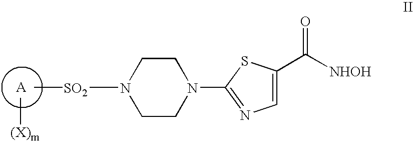
wherein:
A is selected from the group consisting of aryl and heteroaryl;
X is selected from the group consisting of acyl, acylamino, alkyl, substituted alkyl, alkoxy, substituted alkoxy, amino, substituted amino, aminoacyl, aryloxy, substituted aryloxy, cyano, halo, heterocyclic, substituted heterocyclic, nitro, thioalkyl, substituted thioalkyl, and R²—S(O)₂(NH)ₙ— where R² is selected from the group consisting of alkyl, substituted alkyl, aryl, substituted aryl, heteroaryl and substituted heteroaryl;
m is zero, one, two or three; and
n is zero or one;
with the proviso that -A-(X)ₘ is not 4-aminophenyl.

Preferred examples of substituents defined by the formula: 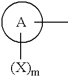
include by way of example only, phenyl, naphthyl, 3-methoxyphenyl, 4-methoxyphenyl, 3,4-dimethoxyphenyl, 4-trifluoromethoxyphenyl, 2-trifluroromethylphenyl, 3-trifluroromethylphenyl, 4-trifluroromethylphenyl, 4-nitrophenyl, 4-acetylphenyl, 4-[(N-morpholino)methyl]phenyl, 4-[(N-pyrrolidinyl)methyl]phenyl, 4-(N,N-dimethylaminomethyl)phenyl, 5-(N,N-dimethylamino)naphthyl, 4-pyrrolind-1-ylphenyl, 4-acetamidophenyl, 4-methyl-2,3-dihydrobenzisoxazinyl, 2,3-dihydrobenzofuran-5-yl, 2,1,3-benzothiadiazol-5-yl, 2,3-dihydro-1,4-benzodioxin-6-yl, 2-chlorophenyl, 2-chloro-6-methylphenyl, 3-chloro-2,5-dimethylphenyl, 3-chlorophenyl, 3,4-dichlorophenyl, 3-chloro-2-methylphenyl, 3-chloro-4-methylphenyl, 4-chlorophenyl, 3-cyanophenyl, 2-fluorophenyl, 3-fluorophenyl, 3,4-difluorophenyl, 3,5-difluorophenyl, 4-fluorophenyl, 4-fluoro-2-methylphenyl, 3-fluoro-4-methylphenyl, 5-fluoro-2-methylphenyl, 4-methylsulfonylphenyl, 2-methylphenyl, 3-methylphenyl, 3-hydroxymethyl-phenyl, 3-(N,N-dimethylaminomethyl)phenyl, 3-(pyrrolidin-1-yl)methylphenyl, 4-ethylphenyl, 4-methylphenyl, 3,4-dimethylphenyl, 3,5-dimethylphenyl, 4-iso-propylphenyl, 4-n-propylphenyl, t-butylphenyl, thien-2-yl, pyrid-2-yl, pyrid-3-yl, benzothiofuran-2-yl, 3,5-dimethylisoxazol-4-yl, 2-(4-morpholino)pyrid-5-yl, and 2-phenoxypyrid-5-yl.

Another preferred aspect of this invention is directed to compounds of formula III as follows: 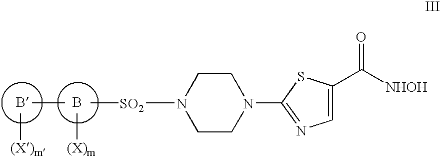
wherein:
B and B′ are independently selected from the group consisting of aryl and heteroaryl;
X and X′ are independently selected from the group consisting of acyl, acylamino, alkyl, substituted alkyl, alkoxy, substituted alkoxy, amino, substituted amino, aminoacyl, aryloxy, substituted aryloxy, cyano, halo, heteroaryl, substituted heteroaryl, heterocyclic, substituted heterocyclic, nitro, thioalkyl, substituted thioalkyl, R²—S(O)₂(NH)ₙ—, where R² is selected from the group consisting of alkyl, substituted alkyl, aryl, substituted aryl, heteroaryl and substituted heteroaryl, and
m is zero, one, two or three;
m′ is zero, one or two; and
n is zero or one.

Preferably, m is zero and m′ is one. When m′ is one, X′ is preferably substituted alkyl and more preferably is represented by the formula:
—CR³R⁴NR⁵R⁶
wherein R³, R⁴, R⁵, or R⁶ are independently hydrogen, alkyl, substituted alkyl, aryl, substituted aryl, heteroaryl and substituted heteroaryl or R⁵ and R⁶ together along with N form a heterocylic or substituted heterocyclic ring containing 3-10 atoms.

Preferred examples of substituents defined by the formula: 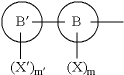
include, by way of example only, (4-pyrazol-1-yl)phenyl, 3-biphenyl, 4-biphenyl, 4-(3-N,N-dimethylaminomethylphenyl)phenyl, 4-(3-N,N-dimethylaminophenyl)phenyl, 4-[(3-pyrrolind-N-ylmethyl)phenyl]phenyl, 4-[(N-morpholinocarbonyl)phenyl]phenyl, 4-(N,N-dimethylaminocarbonyl-phenyl)phenyl, 4-(4-fluorophenyl)phenyl, 4-(pyrid-4-yl)phenyl, 4-(3-chloro-phenyl)phenyl, 4-(2-chlorophenyl)phenyl, 4-(3-fluorophenyl)phenyl, 4-(2-furanyl)phenyl, 4-[2-(pyrrrolidin-N-ylmethyl)thien-3-yl]phenyl, 4-[5-(pyrrrolidin-N-ylmethyl)thien-2-yl]phenyl, 4-[3-(pyrrrolidin-N-ylmethyl)thien-2-yl]phenyl, 4-[3-(4-methylpiperidin-1-yl)phenyl]phenyl, 4-[(3-(N-methyl-N-{2-N,N-dimethyleth-1-yl}aminomethyl)phenyl]phenyl, 4-[(3-(N-methyl-N-ethylaminomethyl)phenyl]phenyl, 4-[(3-(N-methyl-N-isopropyl}aminomethyl)phenyl]phenyl, 4-(methylsulfonamidophenyl)phenyl, 4-[1,3-(benzodioxol-5-yl)]phenyl, 4-(pyrimidin-5-yl)phenyl, 2-(2-methyl-thiopyrimidin-4-yl)thien-5-yl, 4-[4-(acetamidophenyl)]phenyl, 4-(2-N,N-dimethylaminomethyl-thien-3-yl)phenyl, and 4-(pyrid-3-yl)phenyl.

In one of its pharmaceutical composition aspect, this invention is directed to a pharmaceutical composition comprising an effective amount of one or more compounds according to formula I, II and/or III and a pharmaceutically inert carrier.

In another of its pharmaceutical aspects, this invention is directed to pharmaceutical compositions comprising an effective amount of one or more compounds according to formula I, II and/or III and an effective amount of at least one anti-cancer agent, and a pharmaceutically inert carrier.

In one of its method aspects, this invention is directed to a method for inhibiting a proliferative disorder in a mammalian patient which method comprises administering to said patient a pharmaceutical composition comprising a pharmaceutically acceptable carrier and a therapeutically effective amount of one or more compounds of formula I, II and/or III.

In another of its method aspects, this invention is directed to a method for inhibiting a proliferative disorder in a mammalian patient which method comprises administering to said patient a pharmaceutical composition comprising a pharmaceutically acceptable carrier, an effective amount of at least one anti-cancer agent, and a therapeutically effective amount of one or more compounds of formula I, II and/or III.

In yet another of its method aspects, this invention is directed to a method for inhibiting a proliferative disorder in a mammalian patient which method comprises administering to said patient a pharmaceutical composition comprising a pharmaceutically acceptable carrier and a therapeutically effective amount of a compound of formula I-III or a mixture thereof in combination with at least one anti-cancer agent.

For the treatment of the above conditions, the compounds of the invention may be advantageously employed in combination with one or more other medicinal agents, more particularly, with other anti-cancer agents. Examples of anti-cancer agents are: platinum coordination compounds, for example, cisplatin, carboplatin or oxalyplatin; taxane compounds, for example, paclitaxel or docetaxel; topoisomerase I inhibitors such as camptothecin compounds, for example, irinotecan or topotecan; topoisomerase II inhibitors such as anti-tumour podophyllotoxin derivatives, for example, etoposide or teniposide; anti-tumour vinca alkaloids, for example, vinblastine, vincristine or vinorelbine; anti-tumor nucleoside derivatives, for example, 5-fluorouracil, gemcitabine or capecitabine; alkylating agents such as nitrogen mustard or nitrosourea, for example, cyclophosphamide, chlorambucil, carmustine or lomustine; anti-tumour anthracycline derivatives, for example, daunorubicin, doxorubicin, idarubicin or mitoxantrone; HER2 antibodies, for example, trastuzumab; estrogen receptor antagonists or selective estrogen receptor modulators, for example, tamoxifen, toremifene, droloxifene, faslodex or raloxifene; aromatase inhibitors such as exemestane, anastrozole, letrazole and vorozole; differentiating agents such as retinoids, vitamin D and retinoic acid metabolism blocking agents (RAMBA), for example, accutane; DNA methyl transferase inhibitors, for example, azacytidine; kinase inhibitors, for example, flavoperidol, imatinib mesylate or gefitinib; farnesyltransferase inhibitors; or other HDAC inhibitors.

In another of its method aspects, this invention is directed to a method for treating a mammalian patient with one or more diseases or disorders including hematological disorders, e.g., hemoglobinopathies (thalassemias, sickle cell anemias); autosomal dominant disorders, e.g., spinal muscular atrophy and Huntington's disease; genetic related metabolic disorders, e.g., cystic fibrosis and adrenoleukodystrophy; psoriasis; fibrosis, e.g., liver fibrosis, cirrhosis and fibrotic skin diseases, e.g., hypertrophic scars, keloid and Dupuytren's contracture; autoimmune diseases, e.g., systemic lupus erythematosus; acute or chronic degenerative conditions or diseases of the eye, e.g., glaucoma, dry age-related macular degeneration, retinitis pigmentosa and other forms of heredodegenerative retinal disease, retinal detachment and tears; macular pucker, ischemia affecting the outer retina, cellular damage associated with diabetic retinopathy and retinal ischemia, damage associated with laser therapy (grid, focal, and panretinal) including photodyramic therapy, trauma, surgical (retinal translocation, subretinal surgery, or vitrectomy) or light-induced iatrogenic retinopathy, and preservation of retinal transplants; ocular neovascular or edematous diseases and disorders, e.g., diabetic retinopathy, rubeosis iritis, uveitis, Fuch's heterochromatic iridocyclitis, neovascular glaucoma, corneal neovascularization, neovascularization resulting from combined vitrectomy and lensectomy, retinal ischemia, choroidal vascular insufficinency, choroidal thrombosis, carotid artery ischemia, contusive ocular injury, retinopathy of permaturity, retinal vein occlusion, proliferative vitreoretinopathy, corneal angiogenesis, retinal microvasculopathy, or retinal eduema; connective tissue disease, e.g., rheumatoid arthritis, progressive systemic sclerosis, sjorgren's syndrome, dermatomyositis or mixed connective tissue disease; cardiac hypertrophy and heart failure; insulin resistance; amyotrophic lateral sclerosis; multiple sclerosis; Alzheimer's disease; neurodegenerative diseases; and lung diseases, e.g., cystic fibrosis, chronic obstructive pulmonary disease, asthma or acute and chronic bronchitis. Such methods comprise administering to said patient a pharmaceutical composition comprising a pharmaceutically acceptable carrier and a therapeutically effective amount of one or more compounds of formula I, II and/or III.

Preferred compounds of this invention include those found in the Tables below:

TABLE I

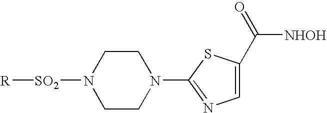

| R | Name |
|---|---|
| 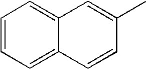 | 2-[4-(naphtha-2-yl-4-sulfonyl)-piperazin-1-yl]- thiazole-5-carboxylic acid hydroxyamide |
| 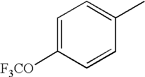 | 2-[4-(4-trifluoromethoxy-benzene sulfonyl)- piperazin-1-yl]-thiazole-5-carboxylic acid hydroxyamide |
| 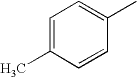 | 2-[4-(4-toluene-4-sulfonyl)-piperazin-1-yl]- thiazole-5-carboxylic acid hydroxyamide |
| 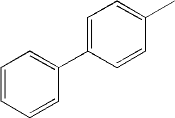 | 2-[4-(biphenyl-4-sulfonyl)-piperazin-1-yl]- thiazole-5-carboxylic acid Hydroxyamide |
| 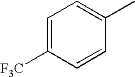 | 2-[4-(4-trifluoromethyl-benzenesulfonyl)- piperazin-1-yl]-thiazole-5-carboxylic acid hydroxyamide |
| 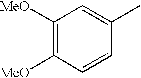 | 2-[4-(3,4-dimethoxy-benzene sulfonyl)-piperazin- 1-yl]-thiazole-5-carboxylic acid hydroxyamide |
| 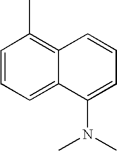 | 2-[4-(5-dimethylamino-naphthalene-1-sulfonyl)- piperazin-1-yl]-thiazole-5-carboxylic acid hydroxyamide |
| 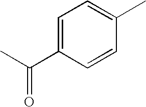 | 2-[4-(4-acetyl-benzenesulfonyl)-piperazin-1-yl]thiazole-5-carboxylic acid hydroxyamide |
| 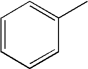 | 2-[4-(benzenesulfonyl)-piperazin-1-yl]-thiazole-5- carboxylic acid hydroxyamide |
| 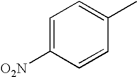 | 2-[4-(4-nitro-benzenesulfonyl)-piperazin-1-yl]- thiazole-5-carboxylic acid hydroxyamide |
| 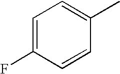 | 2-[4-(4-fluoro-benzenesulfonyl)-piperazin-1-yl]- thiazole-5-carboxylic acid hydroxyamide |
| 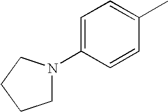 | 2-[4-(4-pyrolidinylbenzenesulfonyl)-piperazin-1- yl]-thiazole-5-carboxylic acid hydroxyamide |
| 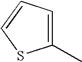 | 2-[4-(thiophene-2-benzenesulfonyl)-piperazin-1- yl]-thiazole-5-carboxylic acid hydroxyamide |
| 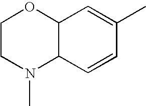 | 2-[4-(N-methyl-2,3- dihydrobenzisoxazinylsulfonyl)-piperazin-1-yl]thiazole-5-carboxylic acid hydroxyamide |
| 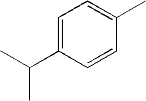 | 2-[4-(4-isopropylphenylsulfonyl)-piperazin-1-yl]- thiazole-5-carboxylic acid hydroxyamide |
| 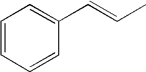 | 2-[4-(trans-2-phenylethanelsulfonyl)-piperazin-1- yl]-thiazole-5-carboxylic acid hydroxyamide |
| 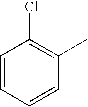 | 2-[4-(2-chlorophenylsulfonyl)-piperazin-1-yl]- thiazole-5-carboxylic acid hydroxyamide |
| 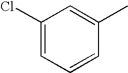 | 2-[4-(3-chlorophenylsulfonyl)-piperazin-1-yl]- thiazole-5-carboxylic acid hydroxyamide |
| 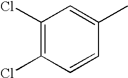 | 2-[4-(3,4-dichlorophenylsulfonyl)-piperazin-1-yl]- thiazole-5-carboxylic acid hydroxyamide |
| 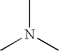 | 2-[4-(N,N-dimethylsulfonyl)-piperazin-1-yl]- thiazole-5-carboxylic acid hydroxyamide |
| 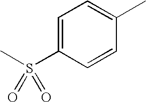 | 2-[4-(4-methylsufonylphenylsulfonyl)-piperazin- 1-yl]-thiazole-5-carboxylic acid hydroxyamide |
| 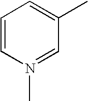 | 2[4-(pyridine-3-sulfonyl)-piperazin-1-yl]thiazole-5-carboxylic acid hydroxyamide |
| 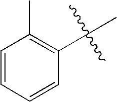 | 2-[4-(2-methylphenylsulfonyl)-piperazin-1-yl]- thiazole-5-carboxylic acid hydroxyamide |
| 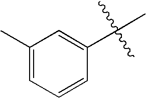 | 2-[4-(3-methylphenylsulfonyl)-piperazin-1-yl]- thiazole-5-carboxylic acid hydroxyamide |
| 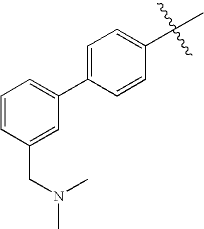 | 2-{4-[(3′-[(dimethylamino)methy 1]-1,1′- biphenylsulfonyl]piperazin-1-yl}-1,3-thiazole-5- carboxylic acid hydroxyamide |
| 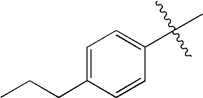 | 2-[4-(4-n-propylphenylsulfonyl)-piperazin-1-yl]- thiazole-5-carboxylic acid hydroxyamide |
| 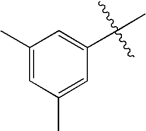 | 2-[4-(3,5-dimethylphenylsulfonyl)-piperazin-1- yl]-thiazole-5-carboxylic acid hydroxyamide |
| 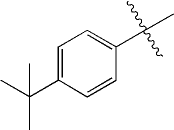 | 2-[4-(4-t-butylphenylsulfonyl)-piperazin-1-yl]- thiazole-5-carboxylic acid hydroxyamide |
| 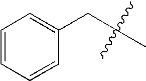 | 2-[4-(benzylsulfonyl)-piperazin-1-yl]-thiazole-5- carboxylic acid hydroxyamide |
| 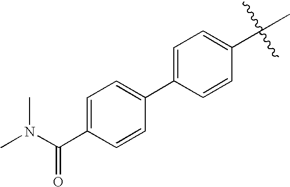 | 2-[4-(4′-N,N-dimethylcarboxamido-1,1′- biphenylsulfonyl)piperazin-1-yl]-1,3-thiazole-5- carboxylic acid hydroxyamide |
| 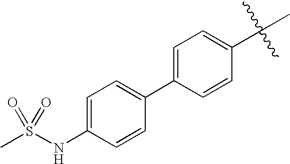 | 2-[4-(4′-methylsulfonylamino-1,1′- biphenylsulfonyl)piperazin-1-yl]-1,3-thiazole-5- carboxylic acid hydroxyamide |
| 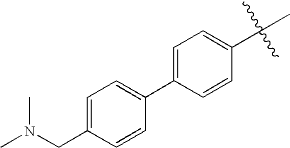 | 2-{4-[(4′-((dimethylamino)methyl)-1,1′- biphenylsulfonyl]piperazin-1-yl}-1,3-thiazole-5- carboxylic acid hydroxyamide |
| 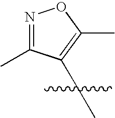 | 2-[4-(3,5-dimethylisoxazolesulfonyl)-piperazin-1- yl]-thiazole-5-carboxylic acid hydroxyamide |
| 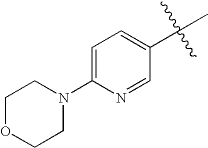 | 2-[4-({4-morpholino}-3-pyridylsulfonyl)- piperazin-1-yl]-thiazole-5-carboxylic acid hydroxyamide |
| 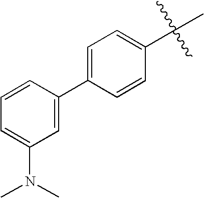 | 2-[4-(3′-(dimethylamino)-1,1′- biphenylsulfonyl)piperazin-1-yl]-1,3-thiazole-5- carboxylic acid hydroxyamide |
| 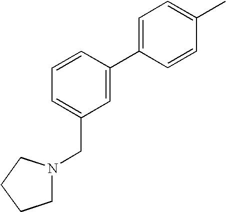 | 2-{4-[(3′-(pyrrolidin-1-ylmethyl)-1,1′- biphenylsulfonyl]piperazin-1-yl}-1,3-thiazole-5- carboxylic acid hydroxyamide |
| 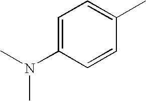 | 2-[4-(4-dimethylaminophenylsulfonyl)-piperazin- 1-yl]-thiazole-5-carboxylic acid hydroxyamide |
| 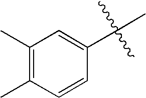 | 2-[4-(3,4-dimethylphenylsulfonyl)-piperazin-1- yl]-thiazole-5-carboxylic acid hydroxyamide |
| 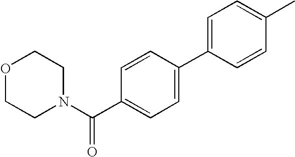 | 2-[4-(4′-(morpholin-4-ylcarbonyl)-1,1′- biphenylsulfonyl)piperazin-1-yl]-1,3-thiazole-5- carboxylic acid hydroxyamide |
| 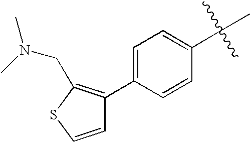 | 2-(4-{4-[(2-((dimethylamino)-methyl)thien-3- yl]phenylsulfonyl}piperazin-1-yl)-1,3-thiazole-5- carboxylic acid hydroxyamide |
| 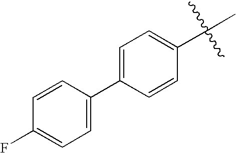 | 2-{4-[(4′-fluoro-1,1′-biphenyl-4- yl)sulfonyl]piperazin-1-yl}-1,3-thiazole-5- carboxylic acid hydroxyamide |
| 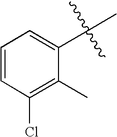 | 2-[4-(3-chloro-2-methylphenylsulfonyl)- piperazin-1-yl]-thiazole-5-carboxylic acid hydroxyamide |
| 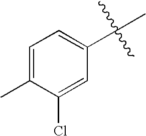 | 2-[4-(3-chloro-4-methylphenylsulfonyl)- piperazin-1-yl]-thiazole-5-carboxylic acid hydroxyamide |
| 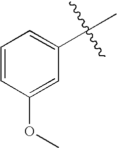 | 2-[4-(4-methoxyphenylsulfonyl)-piperazin-1-yl]thiazole-5-carboxylic acid hydroxyamide |
| 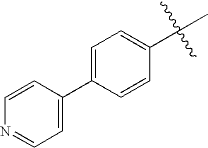 | 2-{4-[4-(pyridin-4-yl)phenylsulfonyl]piperazin-1- yl}-1,3-thiazole-5-carboxylic acid hydroxyamide |
| 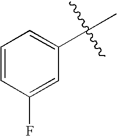 | 2-[4-(3-fluorophenylsulfonyl)-piperazin-1-yl]- thiazole-5-carboxylic acid hydroxyamide |
| 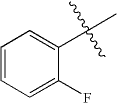 | 2-[4-(2-fluorophenylsulfonyl)-piperazin-1-yl]- thiazole-5-carboxylic acid hydroxyamide |
| 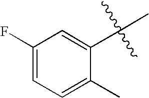 | 2-[4-(2-methyl-5-fluorophenylsulfonyl)-piperazin- 1-yl]-thiazole-5-carboxylic acid hydroxyamide |
| 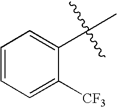 | 2-[4-(2-trifluoromethylphenylsulfonyl)-piperazin- 1-yl]-thiazole-5-carboxylic acid hydroxyamide |
| 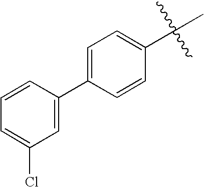 | 2-{4-[(3′-chloro-1,1′-biphenyl-4- yl)sulfonyl]piperazin-1-yl}-1,3-thiazole-5- carboxylic acid hydroxyamide |
| 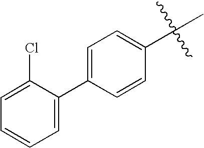 | 2-{4-[(2′-chloro-1,1′-biphenyl-4- yl)sulfonyl]piperazin-1-yl}-1,3-thiazole-5- carboxylic acid hydroxyamide |
| 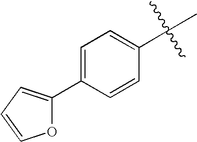 | 2-(4-{[4-(2-furyl)phenyl]sulfonyl}piperazin-1-yl)- 1,3-thiazole-5-carboxylic acid hydroxyamide |
| 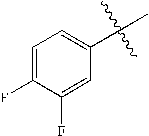 | 2-[4-(3,4-difluorophenylsulfonyl)-piperazin-1-yl]thiazole-5-carboxylic acid hydroxyamide |
| 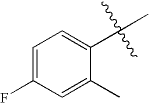 | 2-[4-(4-fluoro-2-methylphenylsulfonyl)-piperazin- 1-yl]-thiazole-5-carboxylic acid hydroxyamide |
| 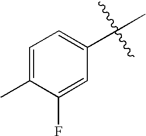 | 2-[4-(3-fluoro-4-methylphenylsulfonyl)-piperazin- 1-yl]-thiazole-5-carboxylic acid hydroxyamide |
| 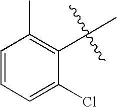 | 2-[4-(2-methyl-6-chlorophenylsulfonyl)- piperazin-1-yl]-thiazole-5-carboxylic acid hydroxyamide |
| 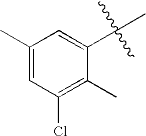 | 2-[4-(2,5-dimethyl-4-chlorophenylsulfonyl)- piperazin-1-yl]-thiazole-5-carboxylic acid hydroxyamide |
| 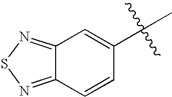 | 2-[4-(2,1,3-benzothiadiazole-5-sulfonyl)- piperazin-1-yl]-thiazole-5-carboxylic acid hydroxyamide |
| 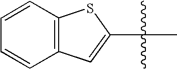 | 2-[4-(2-benzothiphenesulfonyl)-piperazin-1-yl]- thiazole-5-carboxylic acid hydroxyamide |
| 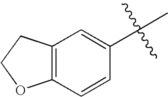 | 2-[4-(2,3-dihydrobenzofuransulfonyl)-piperazin- 1-yl]-thiazole-5-carboxylic acid hydroxyamide |
| 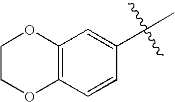 | 2-[4-(3,4-benzodioxansulfonyl)-piperazin-1-yl]- thiazole-5-carboxylic acid hydroxyamide |
| 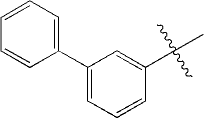 | 2-[4-(3-biphenylsulfonyl)-piperazin-1-yl]- thiazole-5-carboxylic acid hydroxyamide |
| 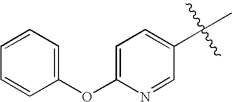 | 2-[4-(2-phenoxypyridine-5-sulfonyl)-piperazin-1- yl]-thiazole-5-carboxylic acid hydroxyamide |
| 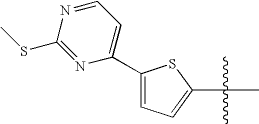 | 2-[4-(2-{2-methylthiopyrimidine-4-yl}-5- thiophenesulfonyl)-piperazin-1-yl]-thiazole-5- carboxylic acid hydroxyamide |
| 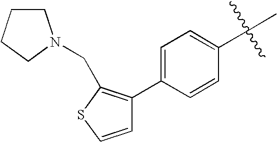 | 2-(4-[4-{(2-(pyrrolidin-1-ylmethyl)-thien-3- yl)phenylsulfonyl}]piperazin-1-yl)-1,3-thiazole-5- carboxylic acid hydroxyamide |
| 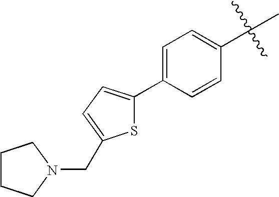 | 2-(4-{4-[(5-pyrrolidin-1-ylmethyl)-thien-2- yl]phenylsulfonyl}piperazin-1-yl)-1,3-thiazole-5- carboxylic acid hydroxyamide |
| 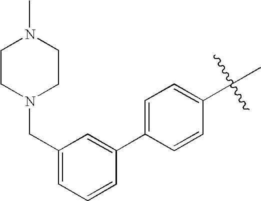 | 2-(4-[(3′-((4-methylpiperazin-1-yl)methyl)-1,1′- biphenylsulfonyl]piperazin-1-yl}-1,3-thiazole-5- carboxylic acid hydroxyamide |
| 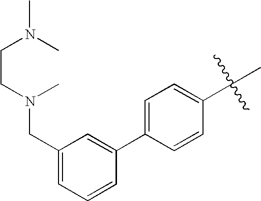 | 2-{4-[3′-{[(2- (dimethylamino)ethyl)(methyl)amino]methyl}- 1,1′-biphenylsulfonyl]piperazin-1-yl}-1,3- thiazole-5-carboxylic acid hydroxyamide |
| 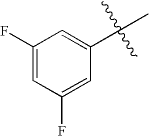 | 2-[4-(3,5-difluorophenylsulfonyl)-piperazin-1-yl]- thiazole-5-carboxylic acid hydroxyamide |
| 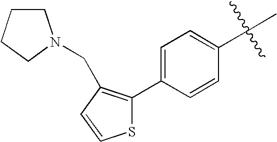 | 2-(4-{4-[(3-(pyrrolidin-1-ylmethyl)-thien-2- yl]phenylsulfonyl}piperazin-1-yl)-1,3-thiazole-5- carboxylic acid hydroxyamide |
| 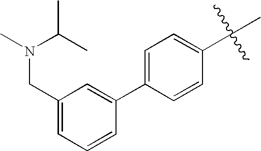 | 2-(4-[(3′-[isopropyl(methyl)amino]methyl]-1,1′- biphenylsulfonyl]piperazin-1-yl}-1,3-thiazole-5- carboxylic acid hydroxyamide |
| 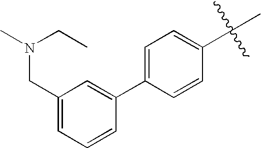 | 2-{4-[(3′-[ethyl(methyl)amino]methyl]-1,1′- biphenylsulfonyl]piperazin-1-yl}-1,3-thiazole-5- carboxylic acid hydroxyamide |
| 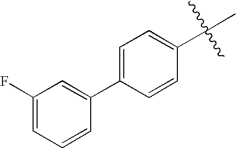 | 2-{4-[(3′-fluoro-1,1′-biphenylsulfonyl]piperazin- 1-yl}-1,3-thiazole-5-carboxylic acid hydroxyamide |
| 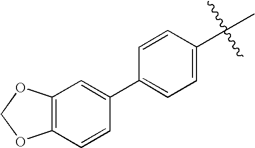 | 2-(4-{[4-(1,3-benzodioxol-5- yl)phenyl]sulfonyl}piperazin-1-yl)-1,3-thiazole-5- carboxylic acid hydroxyamide |
| 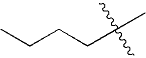 | 2-[4-(n-butylsulfonyl)-piperazin-1-yl]-thiazole-5- carboxylic acid hydroxyamide |
| 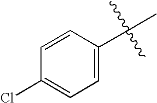 | 2-[4-(chlorophenylsulfonyl)-piperazin-1-yl]thiazole-5-carboxylic acid hydroxyamide |
| 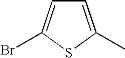 | 2-{4-[(5-bromothien-2-yl)sulfonyl]piperazin-1- yl}-N-hydroxy-1,3-thiazole-5-carboxamide |
| 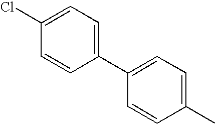 | 2-{4-[(4′-chloro-1,1′-biphenylsulfonyl]piperazin- 1-yl}-1,3-thiazole-5-carboxylic acid hydroxyamide |
| 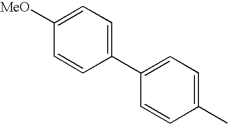 | 2-{4-[(4′-methoxy-1,1′- biphenylsulfonyl]piperazin-1-yl}-1,3-thiazole-5- carboxylic acid hydroxyamide |
| 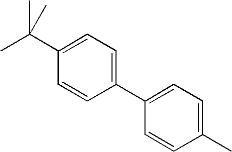 | 2-(4-[(4′-(2,2-dimethylpropyl)-1,1′- biphenylsulfonyl]piperazin-1-yl}-1,3-thiazole-5- carboxylic acid hydroxyamide |
| 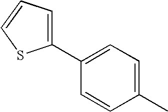 | 2-{4-[(4-thien-2-ylphenyl)sulfonyl]piperazin-1- yl}-1,3-thiazole-5-carboxylic acid hydroxamide |
| 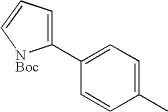 | 2-(4-{[4-(1-(2,2-dimethylprop-oxycarbonyl)-1H- pyrrol-2-yl)phenyl]-sulfonyl}-piperazin-1-yl)-1,3- thiazole-5-carboxylic acid hydroxyamide |
| 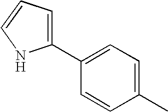 | 2-(4-{[4-(1H-pyrrol-2- yl)phenyl]sulfonyl}piperazin-1-yl)-1,3-thiazole-5- carboxylic acid hydroxyamide |
| 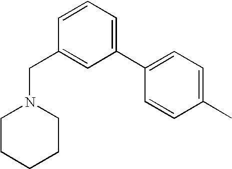 | 2-{4-[(3′-(piperidin-1-ylmethyl)-1,1′- biphenylsulfonyl]piperazin-1-yl}-1,3-thiazole-5- carboxylic acid hydroxyamide |
| 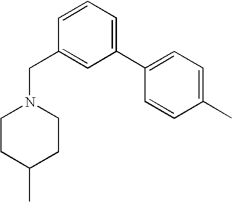 | 2-{4-[(3′-(4-methylpiperidin-1-ylmethyl)-1,1′- biphenylsulfonyl]piperazin-1-yl}-1,3-thiazole-5- carboxylic acid hydroxyamide |
| 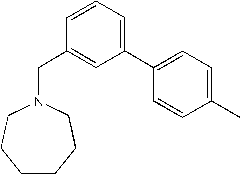 | 2-{4-[(3′-(hexahydroazepin-1-ylmethyl)-1,1′- biphenylsulfonyl]piperazin-1-yl}-1,3-thiazole-5- carboxylic acid hydroxyamide |
| 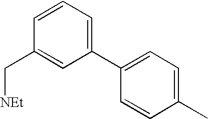 | 2-{4-[(3′-((diethylamino)methyl)-1,1′- biphenylsulfonyl]piperazin-1-yl}-1,3-thiazole-5- carboxylic acid hydroxyamide |
| 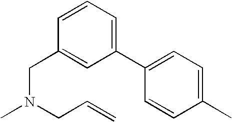 | 2-{4-[(3′-((methyl(3- propenyl)amino)methyl)phenylsulfonyl]piperazin- 1-yl}-1,3-thiazole-5-carboxylic acid hydroxyamide |
| 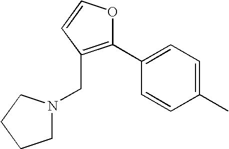 | 2-(4-{4-[(3-(pyrrolidin-1-ylmethyl)-2- furyl]phenylsulfonyl}piperazin-1-yl)-1,3-thiazole- 5-carboxylic acid hydroxyamide |
| 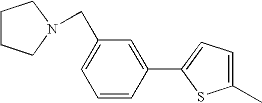 | 2-(4-{5-[3-(pyrrolidin-1- ylmethyl)phenyl]thiophene-2-sulfonyl}piperazin 1-yl)-1,3-thiazole-5-carboxylic acid hydroxyamide |
| 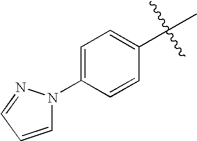 | 2-[4-(4-pyrzaol-1-yl-4-sulfonyl)-piperazin-1-yl]- thiazole-5-carboxylic acid hydmxyamide |
| 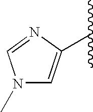 | 2-[4-(4-{1-methylimidazol-4-yl}-4-sulfonyl)- piperazin-1-yl]-thiazole-5-carboxylic acid hydroxyamide |
| 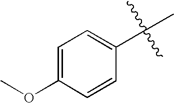 | 2-[4-(4-methoxyphenyl-4-sulfonyl)-piperazin-1- yl]-thiazole-5-carboxylic acid hydroxyamide |
| 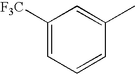 | 2-[4-{3-trifluoromethylphenyl}-4-sulfonyl)- piperazin-1-yl]-thiazole-5-carboxylic acid hydroxyamide |
| 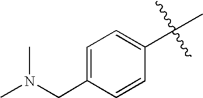 | 2-[4-(4-{N,N-dimethylaminomethylphenyl}-4- sulfonyl)-piperazin-1-yl]-thiazole-5-carboxylic acid hydroxyamide |
| 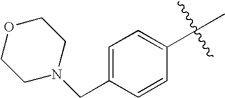 | 2-[4-(4-{N-morpholinomethylphenyl}-4- sulfonyl)-piperazin-1-yl]-thiazole-5-carboxylic acid hydroxyamide |
| 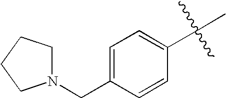 | 2-[4-(4-{N-pyrrolidinylmethylphenyl}-4- sulfonyl)-piperazin-1-yl]-thiazole-5-carboxylic acid hydroxyamide |
| 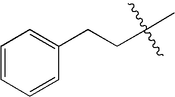 | 2-[4-(4-phenethylphenyl-4-sulfonyl)-piperazin-1- yl]-thiazole-5-carboxylic acid hydroxyamide |
| 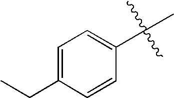 | 2-[4-(4-ethylphenyl-4-sulfonyl)-piperazin-1-yl]- thiazole-5-carboxylic acid hydroxyamide |
| 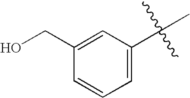 | 2-[4-(3-hydroxymethylphenyl-4-sulfonyl)- piperazin-1-yl]-thiazole-5-carboxylic acid hydroxyamide |
| 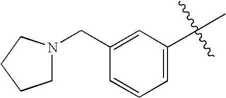 | 2-[4-(3 -pyrrolidin-1-ylmethylphenyl-4-sulfonyl)- piperazin-1-yl]-thiazole-5-carboxylic acid hydroxyamide |
| 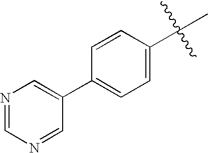 | 2-[4-(4-pyrimid-5-ylphenyl-4-sulfonyl)-piperazin- 1-yl]-thiazole-5-carboxylic acid hydroxyamide |
| 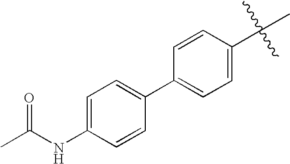 | 2-{4-[(4′-(acetamidophenyl)- phenylsulfonyl]piperazin-1-yl}-1,3-thiazole-5- carboxylic acid hydroxyamide |
| 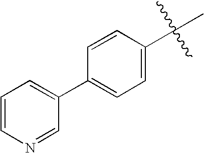 | 2-[4-(3-pyridylphenyl-4-sulfonyl)-piperazin-1-yl]- thiazole-5-carboxylic acid hydroxyamide |
| 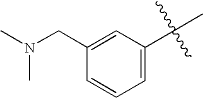 | 2-[4-(3-{N,N-dimethylaminomethylphenyl}-4- sulfonyl)-piperazin-1-yl]-thiazole-5-carboxylic acid hydroxyamide |
| 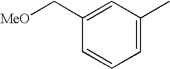 | 2-[4-(3-methoxymethylbenzenesulfonyl)- piperazin-1-yl]-thiazole-5-carboxylic acid hydroxyamide |
| 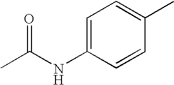 | 2-[4-(4-acetamido-4-sulfonyl)-piperazin-1-yl]thiazole-5-carboxylic acid hydroxyamide |
| 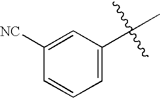 | 2-[4-(3-cyanophenyl}-4-sulfonyl)-piperazin-1-yl]- thiazole-5-carboxylic acid hydroxyamide |

and tautomers, isomers, prodrugs and pharmaceutically acceptable salts thereof.

### DETAILED DESCRIPTION OF THE INVENTION

As noted above, this invention is directed to compounds, pharmaceutical compositions and methods for inhibiting histone deacetylase (HDAC) enzymatic activity. However, prior to describing this invention in more detail, the following terms will first be defined.

Definitions

Unless otherwise limited by a specific recitation herein, the following terms have the following meanings;

“Alkyl” refers to monovalent alkyl groups having from 1 to 10 carbon atoms, preferably from 1 to 5 carbon atoms and more preferably 1 to 3 carbon atoms. This term is exemplified by groups such as methyl, ethyl, n-propyl, iso-propyl, n-butyl, t-butyl, n-pentyl and the like.

“Substituted alkyl” refers to a monovalent alkyl group having from 1 to 3, and preferably 1 to 2, substituents selected from the group consisting of alkoxy, substituted alkoxy, acyl, acylamino, amino, substituted amino, aminoacyl, aryl, substituted aryl, aryloxy, substitutedaryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Alkoxy” refers to the group “alkyl-O-” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O-”.

“Acyl” refers to the groups H—C(O)—, alkyl-C(O)—, substituted alkyl-C(O)—, alkenyl-C(O)—, substituted alkenyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)—.

“Aminoacyl” refers to the group —C(O)NR¹⁰R¹⁰ where each R¹⁰ is independently selected from the group consisting of hydrogen, alkyl, substituted alkyl, alkenyl, substituted alkenyl, aryl, substituted aryl, cycloalkyl, substituted cycloalkyl, heteroaryl, substituted heteroaryl, heterocyclic, substituted heterocyclic and where each R¹⁰ is joined to form together with the nitrogen atom a heterocyclic or substituted heterocyclic ring.

“Alkenyl” refers to a monovalent alkenyl group having from 2 to 6 carbon atoms and more preferably 2 to 4 carbon atoms and having at least 1 and preferably from 1-2 sites of alkenyl unsaturation. The term “alkenyl” encompasses any and all combinations of cis and trans isomers arising from the presence of unsaturation.

“Substituted alkenyl” refers to alkenyl groups having from 1 to 3 substituents, and preferably 1 to 2 substituents, selected from the group consisting of alkoxy, substituted alkoxy, acyl, acylamino, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic provided that any hydroxyl substitution is not on a vinyl carbon atom.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, aryl, substituted aryl, cycloalkyl, substituted cycloalkyl, heteroaryl, substituted heteroaryl, heterocyclic, substituted heterocyclic and where R′ and R″ are joined, together with the nitrogen bound thereto to form a heterocyclic or substituted heterocylic group provided that R′ and R″ are both not hydrogen. When R′ is hydrogen and R″ is alkyl, the substituted amino group is sometimes referred to herein as alkylamino. When R′ and R″ are alkyl, the substituted amino group is sometimes referred to herein as dialkylamino.

“Acylamino” refers to the groups —NR¹¹C(O)alkyl, —NR¹¹C(O)substituted alkyl, —NR¹¹C(O)cycloalkyl, —NR¹¹C(O)substituted cycloalkyl, —NR¹¹C(O)alkenyl, —NR¹¹C(O)substituted alkenyl, —NR¹¹C(O)aryl, —NR¹¹C(O)substituted aryl, —NR¹¹C(O)heteroaryl, —NR¹¹C(O)substituted heteroaryl, —NR¹¹C(O)heterocyclic, and —NR¹¹C(O)substituted heterocyclic where R¹¹ is hydrogen or alkyl.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is to an aromatic ring atom. Preferred aryls include phenyl and naphthyl, e.g, 2-naphthyl.

“Substituted aryl” refers to aryl groups which are substituted with from 1 to 3 substituents, and preferably 1 to 2 substituents, selected from the group consisting of hydroxy, acyl, acylamino, alkyl, substituted alkyl, alkoxy, substituted alkoxy, alkenyl, substituted alkenyl, amino, substituted amino, aminoacyl, aryl, substituted aryl, aryloxy, substituted aryloxy, cycloalkoxy, substituted cycloalkoxy, carboxyl, carboxyl esters, cyano, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Aryloxy” refers to the group aryl-O— that includes, by way of example, phenoxy, naphthoxy, and the like.

“Substituted aryloxy” refers to substituted aryl-O— groups.

“Carboxyl” refers to —COOH or pharmaceutically acceptable salts thereof.

“Carboxyl esters” refers to the groups —C(O)O-alkyl, —C(O)O-substituted alkyl, —C(O)Oaryl, and —C(O)O-substituted aryl wherein alkyl, substituted alkyl, aryl and substituted aryl are as defined herein.

“Cycloalkyl” refers to monovalent cyclic alkyl groups of from 3 to 10 carbon atoms having single or multiple condensed rings which condensed rings may or may not be cycloalkyl provided that the point of attachment is to a cycloalkyl ring atom. Examples of cycloalkyl groups include, by way of example, adamantyl, cyclopropyl, cyclobutyl, cyclopentyl, cyclooctyl and the like.

“Substituted cycloalkyl” refers to a cycloalkyl group, having from 1 to 5 substituents selected from the group consisting of oxo (═O), thioxo (═S), alkyl, substituted alkyl, alkoxy, substituted alkoxy, acyl, acylamino, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Cycloalkoxy” refers to —O-cycloalkyl groups.

“Substituted cycloalkoxy” refers to —O-substituted cycloalkyl groups.

“Halo” or “halogen” refers to fluoro, chloro, bromo and iodo and preferably is fluoro or chloro.

“Heteroaryl” refers to a monovalent aromatic group of from 1 to 15 carbon atoms, preferably from 1 to 10 carbon atoms, and 1 to 4 heteroatoms selected from the group consisting of oxygen, nitrogen, and sulfur within the ring. Optionally, either or both of any nitrogen and/or sulfur atoms within the ring can be oxidized. Such heteroaryl groups can have a single ring (e.g., pyridyl or furyl) or multiple condensed rings (e.g., indolizinyl or benzothienyl) provided that the point of attachment is through a heteroaryl ring atom. Preferred heteroaryls include pyridyl, pyrrolyl, indolyl, thiophenyl, and furyl.

“Substituted heteroaryl” refers to heteroaryl groups that are substituted with from 1 to 3 substituents selected from the same group of substituents defined for substituted aryl.

“Heteroaryloxy” refers to the group —O-heteroaryl and “substituted heteroaryloxy” refers to the group —O-substituted heteroaryl.

“Heterocycle” or “heterocyclic” refers to a monovalent saturated or unsaturated group having a single ring or multiple condensed rings, from 1 to 10 carbon atoms and from 1 to 4 hetero atoms selected from the group consisting of nitrogen, sulfur and oxygen within the ring wherein, in fused ring systems, one or more the rings can be aryl or heteroaryl provided that the point of attachment is to a heterocyclic (non-aromatic) ring atom. Optionally, either or both of any nitrogen and/or sulfur atoms within the ring can be oxidized.

“Substituted heterocyclic” refers to heterocyclic groups that are substituted with from 1 to 3 of the same substituents as defined for substituted cycloalkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydro-benzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

“Thioalkyl” refers to the group —S-alkyl.

“Substituted thioalkyl” refers to the group —S-substituted alkyl.

“Pharmaceutically acceptable salt” refers to pharmaceutically acceptable salts of any of the compounds of Formula I, II, and/or III which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “platinum coordination compound” is used herein to denote any tumor cell growth inhibiting platinum coordination compound which provides platinum in the form of an ion.

The term “taxane compounds” indicates a class of compounds having the taxane ring system and related to or derived form extracts from certain species of yew (Taxus) trees.

The term “topisomerase inhibitors” is used to indicate enzymes that are capable of altering DNA topology in eukaryotic cells. They are critical for important cellular functions and cell proliferation. There are two classes of topoisomerases in eukaryotic cells, namely type I and type II. Topoisomerase I is a monomeric enzyme of approximately 100,000 molecular weight. The enzyme binds to DNA and introduces a transient single-strand break, unwinds the double helix (or allows it to unwind) and subsequently reseals the break before dissociating from the DNA strand. Topisomerase II has similar mechanism of action which involves the introduction of DNA strand breaks of the formation of free radicals.

The term “camptothecin compounds” is used to indicate compounds that are related to or derived from the parent camptothecin compound which is water-insoluble alkaloid derived from the Chinese tree Camptothecin acuminate and the Indian tree Nothapodytes foetida.

The term “podophyllotoxin compounds” is used to indicate compounds that are related to or derived from the parent podophyllotoxin, which is extracted from the mandrake plant.

The term “anti-tumour vinca alkaloids” is used to indicate compounds that are related to or derived from extracts of the periwinkle plant (Vinca rosea).

The term “alkylating agents” encompass a diverse group of chemicals that have the common feature that they have the capacity to contribute, under physiological conditions, alkyl groups to biologically vital macromolecules such as DNA. With most of the more important agents such as the nitrogen mustards and the nitrosoureas, the active alkylating moieties are generated in vivo after complex degradative reactions, some of which are enzymatic. The most important pharmacological actions of the alkylating agents are those that disturb the fundamental mechanisms concerned with cell proliferation in particular DNA synthesis and cell division. The capacity of alkylating agents to interfere with DNA function and integrity in rapidly proliferating tissues provides the basis for their therapeutic applications and for many of their toxic properties.

The term “anti-tumour anthracycline derivatives” comprise antibiotics obtained from the fungus Strep. peuticus var. caesius and their derivatives, characterized by having a tetracycline ring structure with an unusual sugar, daunosamine, attached by a glycosidic linkage.

Amplification of the human epidermal growth factor receptor 2 protein (HER 2) in primary breast carcinomas has been shown to correlate with a poor clinical prognosis for certain patients. Trastuzumab is highly purified recombinant DNA-derived humanized monoclonal IgG1 kappa antibody that binds with high affinity and specificity to the extracellular domain of the HER2 receptor.

Many breast cancers have estrogen receptors and growth of these tumors can be stimulated by estrogen. The terms “estrogen receptor antagonists” and “selective estrogen receptor modulators” are used to indicate competitive inhibitors of estradiol binding to the estrogen receptor (ER). Selective estrogen receptor modulators, when bound to the ER, induces a change in the three-dimensional shape of the receptor, inhibiting its binding to the estrogen responsive element (ERE) on DNA.

In postmenopausal women, the principal source of circulating estrogen is from conversion of adrenal and ovarian androgens (androstenedione and testosterone) to estrogens (estrone and estradiol) by the aromatase enzyme in peripheral tissues. Estrogen deprivation through aromatase inhibition or inactivation is an effective and selective treatment for some postmenopausal patients with hormone-dependent breast cancer.

The term “antiestrogen agent” is used herein to include not only estrogen receptor antagonists and selective estrogen receptor modulators but also aromatase inhibitors as discussed above.

The term “differentiating agents” encompass compounds that can, in various ways, inhibit cell proliferation and induce differentiation. Vitamin D and retinoids are known to play a major role in regulating growth and differentiation of a wide variety of normal and malignant cell types. Retinoic acid metabolism blocking agents (RAMBA's) increase the levels of endogenous retinoic acids by inhibiting the cytochrome P450-mediated catabolism of retinoic acids.

DNA methylation changes are among the most common abnormalities in human neoplasia. Hypermethylation within the promoters of selected genes is usually associated with inactivation of the involved genes. The term “DNA methyl transferase inhibitors” is used to indicate compounds that act through pharmacological inhibition of DNA methyl transferase and reactivation of tumour suppressor gene expression.

The term “kinase inhibitors” comprises potent inhibitors of kinases that are involved in cell cycle progression and programmed cell death (apoptosis).

The term “farnesyltransferase inhibitors” is used to indicate compounds that were designed to prevent farnesylation of Ras and other intracellular proteins. They have been shown to have effect on malignant cell proliferation and survival.

Compound Preparation

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

As to the synthesis of compounds of this invention, Scheme 1 below illustrates a general method for synthesis of compounds of this invention: 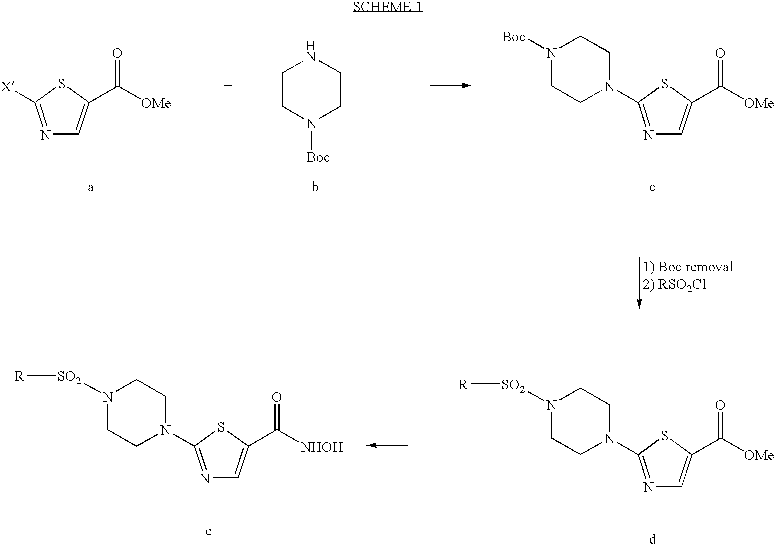
where X′ is a halogen such as bromo or chloro, Pg is a carboxyl protecting group such as an alkyl group (in Scheme 1, methyl) and R is as defined above.

Specifically, commercially available methyl 2-halo-5-carboxylthiazole, compound a, is condensed with at least an equivalent and preferably and excess of mono-protected 1-t-butoxycarbonyl (Boc) piperazine, compound b, under conventional conditions to provide for methyl 2-[(1-t-butoxycarbonyl)piperazin-4-yl]-5-carboxylthiazole, compound c. The reaction is typically conducted in an inert solvent such as acetonitrile, chloroform, and the like in the presence of a suitable base such as potassium carbonate which scavenges the acid generated during the reaction. The reaction is typically conducted at an elevated temperature of from about 40° to 100° C. for a period of time sufficient for substantial completion of the reaction which typically occurs within about 2 to 48 hours. The resulting product, compound c, can be recovered by conventional methods, such as chromatography, filtration, crystallization, evaporation and the like or, alternatively, used in the next step without purification and/or isolation.

Conventional deprotection of the Boc-protected amino group (e.g., TFA) of methyl 2-[(1-t-butoxycarbonyl)piperazin-4-yl]-5-carboxylthiazole, compound c, provides for the corresponding methyl 2-(piperazin-4-yl)-5-carboxylthiazole, not shown, which is then reacted with a suitable sulfonyl chloride (RSO₂Cl) to provide for the corresponding sulfonyl amide, compound d. This latter reaction is typically conducted by combining preferably from about 1.5 to about 2.5 equivalents, of the sulfonyl chloride in an inert diluent such as dichloromethane and the like. Generally, the reaction is conducted at a temperature ranging from about 0° C. to about 40° C. for about 1 to about 24 hours. Preferably, this reaction is conducted in the presence of a suitable base to scavenge the acid generated during the reaction. Suitable bases include, by way of example, tertiary amines, such as triethylamine, diisopropylethylamine, N-methylmorpholine and the like. Alternatively, the reaction can be conducted under Schotten-Baumann-type conditions using aqueous alkali, such as sodium hydroxide and the like, as the base. Upon completion of the reaction, the resulting methyl 2-[1-(R-sulfonylpiperazin-4-yl)]-5-carboxylthiazole, compound d is recovered by conventional methods including neutralization, extraction, precipitation, chromatography, filtration, evaporation and the like.

The sulfonyl chlorides employed in the above reaction are either known compounds or compounds that can be prepared from known compounds by conventional synthetic procedures. Such compounds are typically prepared from the corresponding sulfonic acid, i.e., from compounds of the formula RSO₃H where R is as defined above, using phosphorous trichloride and phosphorous pentachloride. This reaction is generally conducted by contacting the sulfonic acid with about 2 to 5 molar equivalents of phosphorous trichloride and phosphorous pentachloride, either neat or in an inert solvent, such as dichloromethane, at temperature in the range of about 0 to about 80° C. for about 1 to about 48 hours to afford the sulfonyl chloride. Alternatively, the sulfonyl chlorides can be prepared from the corresponding thiol compound, i.e., from compounds of the formula R—SH where R is as defined herein, by treating the thiol with chlorine (Cl₂) and water under conventional reaction conditions.

Examples of sulfonyl chlorides suitable for use in this invention include, but are not limited to, methanesulfonyl chloride, 2-propanesulfonyl chloride, 1-butanesulfonyl chloride, benzenesulfonyl chloride, 1-naphthalene-sulfonyl chloride, 2-naphthalenesulfonyl chloride, p-toluenesulfonyl chloride, 2-methylphenylsulfonyl chloride, 4-acetamidobenzenesulfonyl chloride, 4-tert-butylbenzenesulfonyl chloride, 4-bromobenzenesulfonyl chloride, 2-carboxybenzenesulfonyl chloride, 4-cyanobenzenesulfonyl chloride, 3,4-dichlorobenzenesulfonyl chloride, 3,5-dichlorobenzenesulfonyl chloride, 3,4-dimethoxybenzenesulfonyl chloride, 3,5-ditrifluoromethylbenzenesulfonyl chloride, 4-fluorobenzenesulfonyl chloride, 4-methoxybenzenesulfonyl chloride, 2-methoxycarbonylbenzenesulfonyl chloride, 4-methylamidobenzenesulfonyl chloride, 4-nitrobenzenesulfonyl chloride, 4-thioamidobenzenesulfonyl chloride, 4-trifluoromethyl-benzenesulfonyl chloride, 4-trifluoromethoxybenzenesulfonyl chloride, 2,4,6-trimethylbenzenesulfonyl chloride, 2-phenylethanesulfonyl chloride, 2-thiophenesulfonyl chloride, 5-chloro-2-thiophenesulfonyl chloride, 2,5-dichloro-4-thiophenesulfonyl chloride, 2-thiazolesulfonyl chloride, 2-methyl-4-thiazolesulfonyl chloride, 1-methyl-4-imidazolesulfonyl chloride, 1-methyl-4-pyrazolesulfonyl chloride, 5-chloro-1,3-dimethyl-4-pyrazolesulfonyl chloride, 3-pyridinesulfonyl chloride, 2-pyrimidinesulfonyl chloride and the like. If desired, a sulfonyl fluoride, sulfonyl bromide or sulfonic acid anhydride may be used in place of the sulfonyl chloride in the above reaction to form the N-sulfonyl amino acids.

The methyl carboxyl group of compound d can then be converted to a hydroxyamide by reaction with a 2-20 fold excess of hydroxylamine. The reaction is typically conducted in a suitable diluent such as a 5:2 mixture of methanol to water under basic conditions, e.g, the addition of sodium hydroxide. The reaction is typically conducted at a temperature of from about −20° to 20° C. for a period of time sufficient for substantial completion of the reaction which typically occurs within about 0.5 to 10 hours. The resulting amide, compound e, can be recovered by conventional methods, such as chromatography, filtration, crystallization, evaporation and the like.

Alternatively, an ester d is converted to the hydroxamic acid e as shown in Scheme 2. 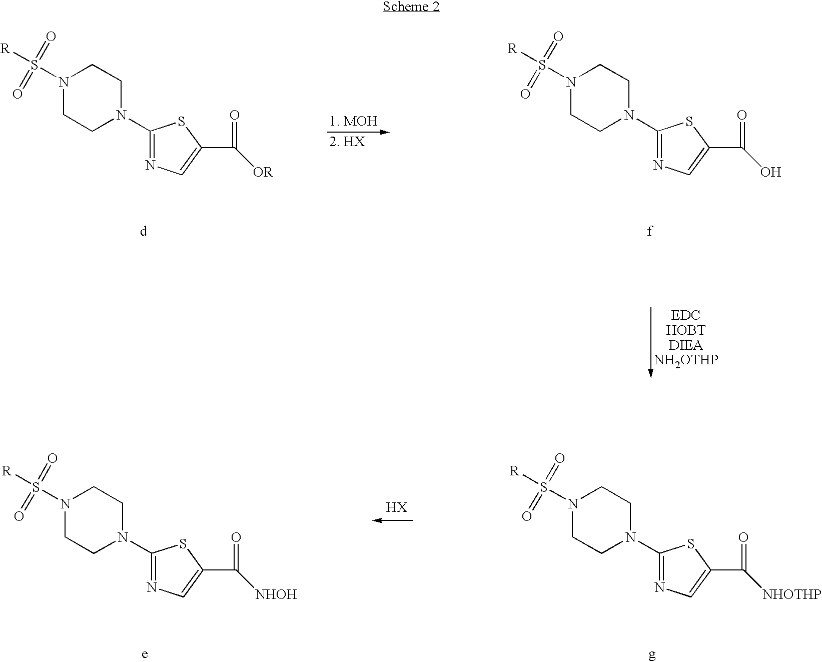
where X is a halo group such as chloro or bromo and M is an alkali metal such as sodium, potassium and the like.

The ester prepared by the methods of Scheme 1 is hydrolyzed to a carboxylic acid f with about 1-20 equivalents of an alkali metal hydroxide in a mixture of water and a suitable organic solvent in about one to 48 hours at about 20 to 100° C. Suitable organic solvents include, but are not limited to, tetrahydrofuran, ethanol, methanol, or dioxane. The reaction mixture is neutralized with an inorganic acid such as hydrochloric, hydrobromic, or sulfuric acid and the solvents are evaporated. The residue is suspended in a suitable solvent and treated with about one to five equivalents of a tertiary amine such as, but not limited to, triethylamine or diisopropylethylamine (DIEA), about one to five equivalents of N-hydroxybenzotriazole (HOBT), and about one to five equivalents of a carbodiimide coupling reagent such as, but not limited to, dicyclohexylcarbodiimide or 1-[3-(dimethylamino)propyl]-1-ethylcarbodiimide (EDC) and about one to five equivalents of O-(tetrahydro-2H-pyran-2-yl)hydroxylamine (NH₂OTHP) for about one to 48 hours at about 20 to 100° C. to produce a protected hydroxamic acid g. A solution of about 1 to 50% strong acid such as, but not limited to, hydrochloric acid or trifluoroacetic acid in an organic solvent such as, but not limited to, dichloromethane, dichloroethane, methanol, ethanol, or dioxane at about 0° to 80° C. in about one minute to 24 hours converts g to the hydroxamic acid e that is recovered by the means previously described.

Scheme 3, below, illustrates an alternative general synthesis of the compounds of this invention of general Formula III. 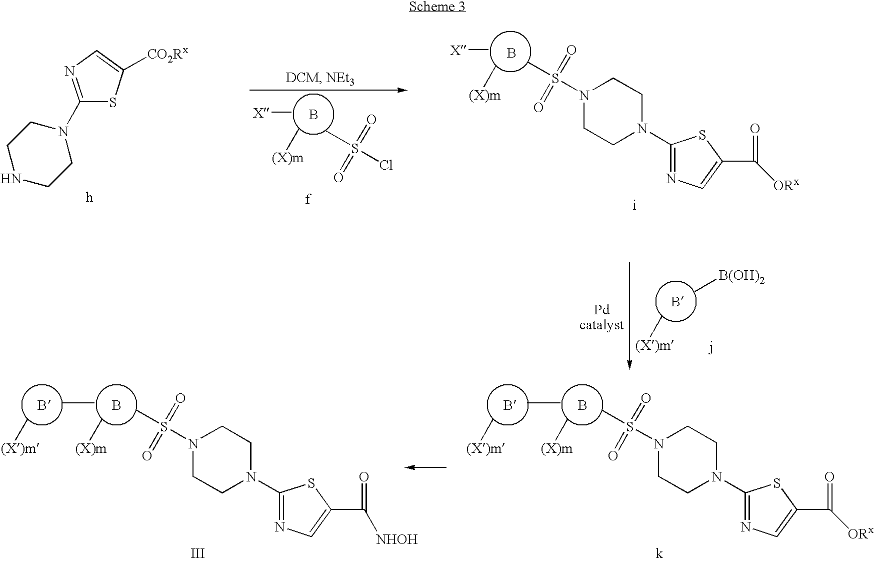
where B, B′, X, X′, m, m′ are as defined, R^{x} is alkyl and X″ is a halo group.

The compounds of Formula III are synthesized wherein an aryl or heteroaryl sulfonamide i, prepared by the methods of Scheme 1, and bearing a halo group X″, preferably chloro, bromo, or iodo, reacts with about one to three equivalents of an aryl or heteroaryl boronic acid i in the presence of about one to three equivalents of a base such as an alkali metal carbonate and about 0.1 to 20 mole percent of a palladium catalyst in a suitable solvent in about one to 72 hours at about 20 to 150° C. to provide substituted biaryl, heteroaryl-aryl, aryl-heteroaryl or heteroaryl-heteroaryl sulfonamides k. The preferred R^{x} groups are methyl and ethyl. Examples of suitable solvents include, but are not limited to, dimethylformamide, dimethylacetamide, dioxane, and tetrahydrofuran. Examples of palladium catalysts include, but are not limited to, diacetoxybis(triphenylphospine)palladium, dichlorobis(triphenylphospine)-palladium, and tetrakis(triphenylphosphine)-palladium. Examples of suitable alkali metal carbonates include, but are not limited to, sodium, potassium or cesium carbonate. Subsequent conversion of the ester k to the hydroxamic acids of Formula III are accomplished by any one of the means described in Scheme 1 or 2.

Scheme 4, below, describes the synthesis of compounds of this invention of Formula III where X or X′ is a group —CR³R⁴NR⁵R⁶ wherein R³, R⁴, R⁵, and R⁶ are as previously defined. Preferably, substituents R³ and R⁴ are hydrogen.

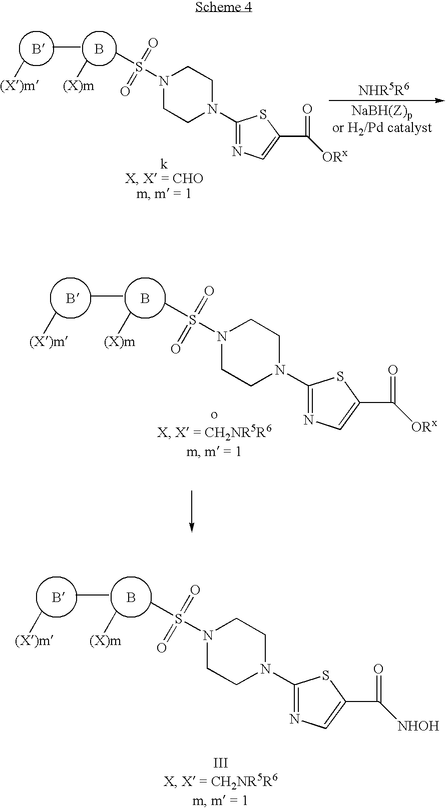

A sulfonamide k, prepared as in Scheme 3, wherein X or X′ is an aldehyde group is reductively aminated with one to 50 equivalents of an amine, NHR⁵R⁶, or hydroxylamine in a suitable solvent at from about 0° to 80° C. for about one to 72 hours in the presence of about one to ten equivalents of a suitable borohydride reducing agent. Alternatively, the suitable borohydride reducing agent can be replaced by about 0.05 to 1 equivalents of a suitable palladium catalyst and about one to ten atmospheres of hydrogen. Preferred R^{x} groups are methyl and ethyl. Suitable solvents include, but or not limited to, methylene chloride, tetrahydrofuran, dioxane, ethanol, trimethylorthoformate, tetramethylorthoformate, ether, dichloroethane, or ethylacetate. Suitable borohydride reducing reagents include, but are not limited to, sodium borohydride, sodium cyanoborohydride, and sodium triacetoxyborohydride. Suitable palladium catalysts include, but are not limited to, palladium on carbon, palladium on alumina, palladium on barium carbonate, or palladium oxide. Subsequent conversion of the ester o to a hydroxamic acid is accomplished by any of the means described in Scheme 1 or 2.

Pharmaceutical Formulations

When employed as pharmaceuticals, the compounds of this invention are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions which contain, as the active ingredient, one or more of the compounds of formula I-VII above associated with pharmaceutically acceptable carriers. In making the compositions of this invention, the active ingredient is usually mixed with an excipient, diluted by an excipient or enclosed within such a carrier which can be in the form of a capsule, sachet, paper or other container. The excipient employed is typically an excipient suitable for administration to human subjects or other mammals. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh. Alternatively, poorly water soluble compounds can be prepared in the form of nanoparticles to enhance their solubility. See, for example, International Patent Application Publication No. WO 03/024424 for “Stabilization of Active Agents by Formulation into Nanoparticulate Form” which is incorporated herein by reference in its entirety.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The compounds of the present invention maybe administered to patients either alone or in combination with other known anti-tumor agents. When administered alone about 0.005 to about 100 mg/kg, more preferably about 0.005 to about 10 mg/kg, are administered to the patient. Higher and lower dosages may be used. Administration may occur once a day, or several times in a day. In addition the treatment may be repeated every 7, 14, 21 or 28 days.

When administered in combination with other anti-cancer agents, the compounds of the present invention may be prepared in a formulation that includes both one or more of the compounds of Formula I-III and one or more other anti-cancer agents. Alternatively the other anti-cancer agents may be administered in a separate formulation which may be administered before, after or simultaneously with the compounds of this invention. When administered in combination with at least one other anti-cancer agent, about 0.005 to about 100 mg/kg, more preferably about 0.5 to about 10 mg/kg, of one or more compounds of this invention are administered to the patient. Higher and lower dosages may be used. The dosages of the other anti-cancer agents are known in the art. Administration may occur once a day, or several times in a day. In addition the treatment may be repeated every 7, 14, 21 or 28 days.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices which deliver the formulation in an appropriate manner.

The following formulation examples illustrate the pharmaceutical compositions of the present invention.

### FORMULATION EXAMPLE 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### FORMULATION EXAMPLE 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### FORMULATION EXAMPLE 3

A dry powder inhaler formulation is prepared containing the following components:

| Ingredient | Weight % |
|---|---|
| Lactose | 5 |
| Active Ingredient | 95 |

The active mixture is mixed with the lactose and the mixture is added to a dry powder inhaling appliance.

### FORMULATION EXAMPLE 4

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

### FORMULATION EXAMPLE 5

Capsules, each containing 40 mg of medicament are made as follows:

| | Quantity | |
|---|---|---|
| Ingredient | (mg/capsule) | |
| Active Ingredient | 40.0 | mg |
| Starch | 109.0 | mg |
| Magnesium stearate | 1.0 | mg |
| Total | 150.0 | mg |

The active ingredient, cellulose, starch, an magnesium stearate are blended, passed through a No. 20 mesh U.S. sieve, and filled into hard gelatin capsules in 150 mg quantities.

### FORMULATION EXAMPLE 6

Suppositories, each containing 25 mg of active ingredient are made as follows:

| Ingredient | Amount | |
|---|---|---|
| Active Ingredient | 25 | mg |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### FORMULATION EXAMPLE 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The medicament, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### FORMULATION EXAMPLE 8

| | Quantity | |
|---|---|---|
| Ingredient | (mg/capsule) | |
| Active Ingredient | 15.0 | mg |
| Starch | 407.0 | mg |
| Magnesium stearate | 3.0 | mg |
| Total | 425.0 | mg |

The active ingredient, cellulose, starch, and magnesium stearate are blended, passed through a No. 20 mesh U.S. sieve, and filled into hard gelatin capsules in 560 mg quantities.

### FORMULATION EXAMPLE 9

An intravenous formulation may be prepared as follows:

| Ingredient | Quantity | |
|---|---|---|
| Active Ingredient | 250.0 | mg |
| Isotonic saline | 1000 | mL |

### FORMULATION EXAMPLE 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermnal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intraarterial infusion of hypertonic solutions which can transiently open the blood-brain barrier.

Utility

Deacetylases are found in transcriptional repression pathways. In addition, histone deacetylases (HDAC) play an important role in cell proliferation and differentiation. Inhibition of histone deacetylation results in cell cycle arrest, cellular differentiation, apoptosis and reversal of the transformed phenotype. Therefore, HDAC inhibitors are useful in the treatment and/or amelioration of cell proliferative diseases or conditions, such as cancers. Other diseases in which said HDAC inhibitors are useful are hematological disorders, e.g., hemoglobinopathies (thalassemias, sickle cell anemias); autosomal dominant disorders, e.g., spinal muscular atrophy and Huntington's disease; genetic related metabolic disorder, e.g., cystic fibrosis and adrenoleukodystrophy (U.S.2004/0029903 A1, U.S. Pat. No. 6,124,495); psoriasis (McLaughlin, F.; La Thangue, N. B., Current Drug Targets-Inflammation, 2004, 3, 213-219); fibrosis, e.g., liver fibrosis, cirrhosis and fibrotic skin diseases, e.g., hypertrophic scars, keloid and Dupuytren's contracture (U.S. Pat. No. 5,993,845); autoimmune diseases, e.g., systemic lupus erythematosus (U.S.2003/0082666 A1); acute or chronic degenerative conditions or diseases of the eye, e.g., glaucoma, dry age-related macular degeneration, retinitis pigmentosa and other forms of heredodegenerative retinal disease, retinal detachment and tears; macular pucker, ischemia affecting the outer retina, cellular damage associated with diabetic retinopathy and retinal ischemia, damage associated with laser therapy (grid, focal, and panretinal) including photodynamic therapy, trauma, surgical (retinal translocation, subretinal surgery, or vitrectomy) or light-induced iatrogenic retinopathy, and preservation of retinal transplants (U.S.2004/0092431 A1); ocular neovascular or edematous diseases and disorders, e.g., diabetic retinopathy, rubeosis iritis, uveitis, Fuch's heterochromatic iridocyclitis, neovascular glaucoma, corneal neovascularization, neovasculariztion resulting from combined vitrectomy and lensectomy, retinal ischemia, choroidal vascular insufficiency, choroidal thrombosis, carotid artery ischemia, contusive ocular injury, retinopathy of prematurity, retinal vein occlusion, proliferative vitreoretinopathy, corneal angiogenesis, retinal microvasculopathy, or retinal edema (U.S. 2004/0092558 A1); connective tissue disease, e.g., rheumatoid arthritis, progressive systemic sclerosis, sjorgren's syndrome, dermatomyositis or mixed connective tissue disease (U.S. 2003/0206946 A1); cardiac hypertrophy and heart failure (U.S. Pat. No. 6,706,686 B2); insulin resistance (U.S. 2004/0058868 A1); amyotrophic lateral sclerosis (U.S. 2004/0077591 A1); multiple sclerosis (U.S. 2004/0077591 A1); Alzheimer's disease (U.S. 2004/0077591 A1); neurodegenerative diseases (U.S. 2004/0087657 A1); and lung diseases, e.g., cystic fibrosis, chronic obstructive pulmonary disease, asthma or acute and chronic bronchitis (U.S. 2004/0167184 A1). Each of the above references are incorporated herein by reference in their entirety.

The following synthetic and biological examples are offered to illustrate this invention and are not to be construed in any way as limiting the scope of this invention. Unless otherwise stated, all temperatures are in degrees Celsius.

### EXAMPLES

In the examples below, the following abbreviations have the following meanings. If an abbreviation is not defined, it has its generally accepted meaning.
bm=broad multiplet
bs=broad singlet
bt=broad triplet
Boc=N-tert-butoxycarbonyl
d=doublet
dd=doublet of doublets
DCM=Dichloromethane
DIEA=diisopropylethylamine
DMEM=Delbaco's minimum eagle's medium
DMF=N,N-dimethylformamide
DMSO=Dimethylsulfoxide
EDC=1-[3-(dimethylaminopropyl]-1-ethylcarbodiimide
EtOAc=ethyl acetate
g=grams
h=hour
HOBt=N-hydroxybenzotriazole
HPLC=high performance liquid chromatography
HPLC %=Percent purity
L=Liter
LCMS or LC/MS=Liquid chromatography/mass spectrum
m=multiplet
M=molar
M+1=molecular weight+1
Me=methyl
MeOH=methanol
min=minutes
mg=milligram
mL=milliliter
mm=millimeter
mM=millimolar
mmol=millimol
MHz=Megahertz
N=normal
nm=nanometers
NMR=nuclear magnetic resonance
m/e or m/z=mass to charge ratio in mass spectrum
q=quartet
q.s.=means adding a quantity sufficient to achieve a certain state
RPHPLC=reverse phase high performance liquid chromatography
rt=room temperature
Rt=retention time
s=singlet
sec=seconds
t=triplet
TFA=Trifluoroacetic acid
THF=tetrahydrofuran
TLC or tlc=thin layer chromatography
w/v=weight to volume
v/v=volume to volume
μL=Microliter
μM=Micromolar
μm=Micron

All the chemicals starting materials were obtained from commercial suppliers and used without further purification.

Flash column chromatography was performed with silica (60-120 mesh). Analytical RPHPLC was done using Shimadzu HPLC equipped with a PDA detector using the following columns and systems: a Thermo Hypersil BDS, 4.6×150 mm, 5 μm particle size, C-18 column, isocratic using acetonitrile:0.1% TFA in water (60:40), flow rate=0.5 mL/min (System-1); Thermo Hypersil BDS, 4.6×250 mm, 5 μm particle size, C-18 column, linear gradient A-acetonitrile: B-0.1% TFA in water; 0.01 min A (10%):B (90%); 5.00 min A (10%):B (90%); 15.00 min A (90%):B (10%); 20.00 min A (90%):B (10%); 25.00 min A (10%):B (90%); 30.00 min A (10%):B (90%); 30.00 min Stop; flow rate=1.5 mL/min (System-2).

¹H NMR spectra were recorded at 200 or 300 MHz and the proton chemical shifts are expressed in ppm relative to internal tetramethylsilane (TMS) and coupling constants (J) are expressed in hertz (Hz). Mass spectra (MS) were carried out using a Micromass model.

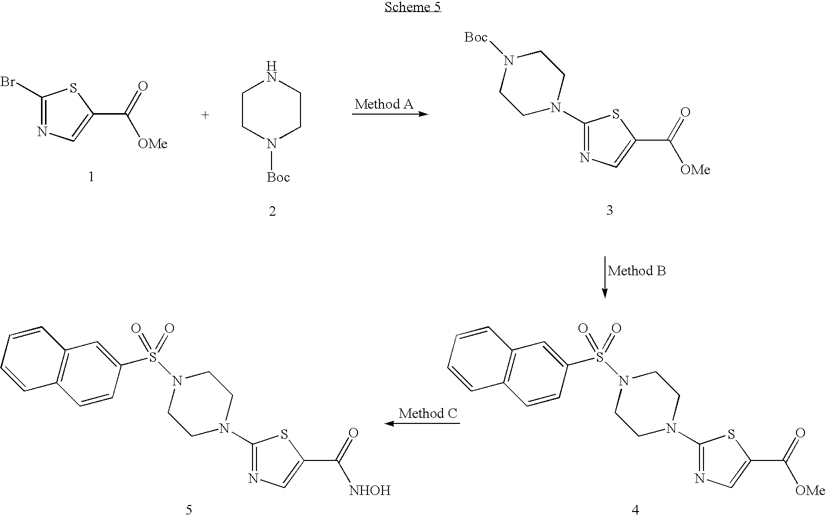

### Example 1

### Synthesis of 2-[4-(naphtha-2-yl-4-sulfonyl)-piperazin-1-yl]-thiazole-5-carboxylic acid hydroxyamide

### Alternative Nomenclature—1-(2-natphthylsulfonyl)-4-(5-hydroxyaminocarbonylthiazol-2-yl) piperazine

Method A: To bromothiazole 1 (1 g, 4.18 mmol) in acetonitrile (40 mL) was added potassium carbonate (1.32 g, 10 mmol) followed by N-Boc piperizine 2 (0.935 g, 5 mmol). The reaction mixture was held at 80° C. for 16 h. At the end of the reaction time, acetonitrile was removed on roto-evaporation and the residue was taken in ethyl acetate (50 mL) and washed with brine (30 mL). The crude product 3 obtained (1.4 g, 99%) on removal of solvent was taken as such for the next reaction.

TLC (Rf): 0.41 (30% EtOAc in hexanes).

¹H NMR (300 MHz, CDCl₃) δ: 7.76 (s, 1), 3.78 (s, 3), 3.66-3.69 (m, 4), 3.19-3.22 (m, 4), 1.49 (s, 9). MS (ES+): 328 (M+1).

Method B: To the crude product 3 obtained from method A (1.4 g, 4.15 mmol) TFA (20%) in dichloromethane was added and stirred at room temperature for an h. After removing the solvent, the residue was kept under high vacuum for 1 h. The residue was then redissolved in DCM (20 mL) to which triethylamine (6.0 mL, 41.5 mmol) and 2-naphthalene sulfonyl chloride (1.85 g, 8.2 mmol) was added and stirred at room temperature over night. Subsequently more DCM (50 mL) was added and washed with 1N hydrochloric acid (20 mL). The crude product obtained on removal of solvent was purified on a column chromatography using ethyl acetate in hexanes (1:1) to obtain product 4 (1.15 g, %) as white crystalline solid.

TLC (Rf): 0.41 (30% EtOAc in hexanes).

¹H NMR (300 MHz, CDCl₃): δ 8.32 (d, J=1.5 Hz, 1H), 7.89-7.98 (m, 4H), 7.76 (s, 1H), 7.70-7.73 (dd, J=1.8, 8.4 Hz, 1H), 7.61-7.66 (m, 2H), 3.78 (s, 3H), 3.66-3.69 (m, 4H), 3.19-3.22 (m, 4H). MS (ES+): 418 (M+1).

Method C: To the product 4 (200 mg, 0.46 mmol) in methanol (5 mL), aqueous hydroxylamine (30 μL, 4.60 mmol, 50% solution) and sodium hydroxide (118 mg, 3.22 mmol, 2 mL) in water (2 mL) was added and the reaction mixture was held at 0° C. for 4 hours. After acidification with 1N HCl, the solvent was removed and the residue was taken up in ethyl acetate and washed with brine. The product 5 obtained (100 mg) on removal of solvent was purified on a RPHPLC.

TLC (Rf): 0.41 (30% EtOAc in hexanes).

¹H NMR (300 MHz, CD₃OD): δ 8.41 (m, 1H), 7.97-8.10 (m, 3H), 7.76-7.80 (dd, J=1.8, 8.4 Hz, 1H), 7.76 (s, 1H), 7.64-7.69 (m, 2H), 3.66-3.69 (m, 4H), 3.23-3.20 (m, 4H). MS m/e: 418 (M+1).

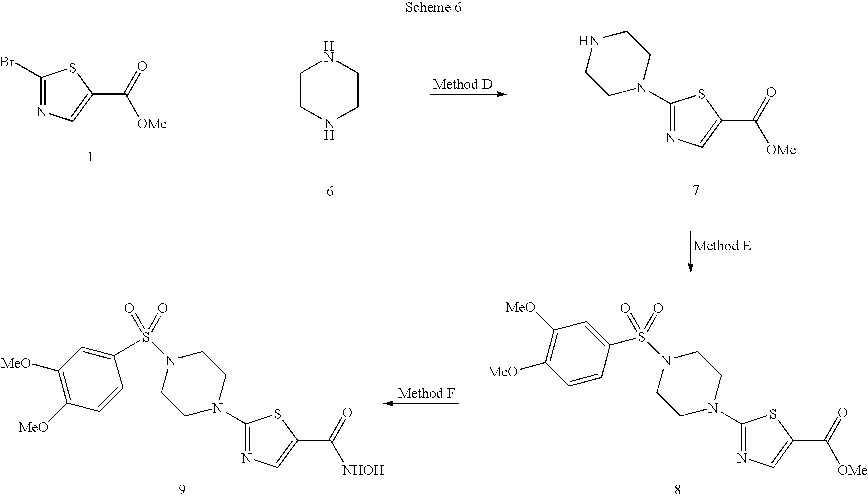

### Preparation of 2-[4-(3,4-dimethoxy-benzene sulfonyl)-piperazin-1-yl]-thiazole-5-carboxylic acid hydroxyamide

Method D: To a solution of methyl 2-bromothiazole-5-carboxylate 1 (5.00 g, 22.50 mmol) in acetonitrile (50 mL) were added piperazine 6 (2.32 g, 26.97 mmol) and potassium carbonate (6.22 g, 45.05 mmol) under a N₂ atmosphere. The reaction mixture was heated to reflux at 80° C. for 10 h. The reaction mixture was filtered through Celite and the filtrate was concentrated under reduced pressure. The residue was purified by column chromatography using silica gel to give 2-piperazin-1-yl-thiazole-5-carboxylic acid methyl ester 7 as a solid (4.10 g, 79.8%).

HPLC: 92% (Rt=3.883 min).

¹H NMR (CDCl₃, 200 MHz) δ=7.88 (1H, s), 3.89 (3H, s), 3.55 (4H, t, J=6.0 Hz), 2.98 (4H, t, J=6.0 Hz). MS (m/z): 228 (M+1).

Method E: To a solution of intermediate 7 (200 mg, 0.886 mmol) in dichloromethane (7.5 mL) were added 3,4-dimethoxybenzenesulfonyl chloride (320 mg, 1.320 mmol) and triethylamine (220 mg, 2.169 mmol) under a N₂ atmosphere. The reaction mixture was stirred at room temperature for 4 h. Water (10 mL) followed by dichloromethane (10 mL) were added to the reaction mixture and the organic layer was separated, dried on sodium sulfate, filtered and concentrated under reduced pressure. The residue was purified by column chromatography using silica gel to give the compound 2-[4-(3,4-dimethoxy-benzene sulfonyl)-piperazin-1-yl]-thiazole-5-carboxylic acid methyl ester (8) as a solid (200 mg, 53.0%).

HPLC: 99% (Rt 6.507 min).

¹H NMR (CDCl₃, 200 MHz): δ=7.82 (1H, s), 7.38 (1H, dd, J=2.2, 8.6 Hz), 7.19 (1H, d, J=2.2 Hz), 6.96 (1H, d, J=8.6 Hz), 3.93 (6H, 2s), 3.83 (3H, s), 3.68 (4H, t, J=5.2 Hz), 3.14 (4H, t, J=5.2 Hz).

MS (m/z): 427 (M+1).

Method F: To a solution of compound 8 (125 mg, 0.292 mmol) in 1,4-dioxane (2 mL) were added hydroxylamine hydrochloride (202 mg, 2.92 mmol) and a freshly prepared solution of sodium methoxide in methanol (100 mg, 4.35 mmol of sodium dissolved in 1 mL of methanol) under N₂ atmosphere. The reaction mixture was stirred at room temperature for 2 h. The reaction mixture was acidified to pH˜6 with 1M HCl and the formed precipitates were filtered off. The filtrate was diluted with ethylacetate (5 mL) and water (2 mL) and the organic layer was separated. The aqueous layer was washed with ethyl acetate (5 mL) and the combined organic layers were dried on sodium sulfate, filtered and concentrated under reduced pressure. The residue was purified by preparative HPLC to give 9 as a solid.

HPLC: 86.09% (Rt=12.51 min).

¹H NMR (CD₃OD, 200 MHz): δ 8.14 (1H, s), 7.61-7.08 (3H, m), 3.83 (6H, s), 3.54 (4H, m), 3.03 (4H, m).

MS m/e: 429 (M+1).

Following the procedures set forth in Example 2 above, the compounds of following examples were prepared using the appropriate starting materials and the ¹H NMR data, HPLC and/or mass spectral data are presented below.

### Example 3

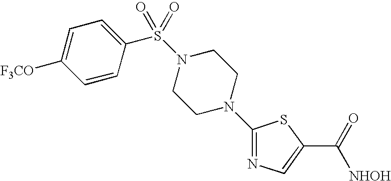

### 2-[4-(4-trifluoromethoxy-benzene sulfonyl)-piperazin-1-yl]-thiazole-5-carboxylic acid hydroxyamide

HPLC: 92% (Rt=14.16 min).

¹H NMR (CD₃OD, 200 MHz): δ 8.00 (3H, m), 7.57 (2H, m), 3.64 (4H, m), 3.19 (4H, m);

MS (m/z): 453 (M+1).

### Example 4

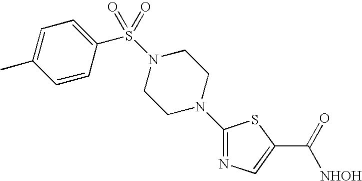

### 2-[4-(4-toluene-4-sulfonyl)-piperazin-1-yl]-thiazole-5-carboxylic acid hydroxyamide

¹H NMR (CD₃OD, 200 MHz): δ 7.79 (3H, m), 7.44 (2H, d, J=8.6 Hz), 3.65 (4H, t, J=5.4 Hz), 3.12 (4H, t, J=5.4 Hz), 2.46 (3H, s).

MS (m/z): 382.6 (M+1).

### Example 5

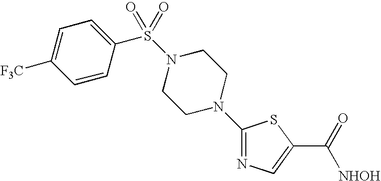

### 2-[4-(4-trifluoromethyl-benzenesulfonyl)-piperazin-1-yl]-thiazole-5-carboxylic acid hydroxyamide

HPLC: 85.42% (Rt=14.11 min).

¹H NMR (CD₃OD, 200 MHz): δ 8.03 (4H, m), 7.81 (1H, s), 3.75 (4H, m), 3.21 (4H, m).

MS (m/z): 436 (M+1).

### Example 6

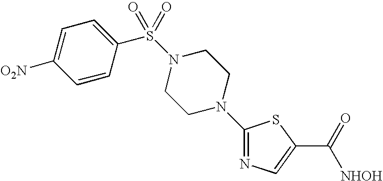

### 2-[4-(4-nitro-benzenesulfonyl)-piperazin-1-yl]-thiazole-5-carboxylic acid hydroxyamide

MS (m/z): 414 (M+1).

### Example 7

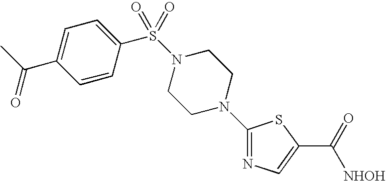

### 2-[4-(4-acetyl-benzenesulfonyl)-piperazin-1-yl]-thiazole-5-carboxylic acid hydroxyamide

HPLC: 92.26% (Rt=12.51 min).

¹H NMR (CD₃OD, 200 MHz): δ 7.86-7.68 (5H, m), 3.64 (4H, m), 3.17 (4H, m), 2.26 (3H, s).

MS (m/z): 445 (M+1).

### Example 8

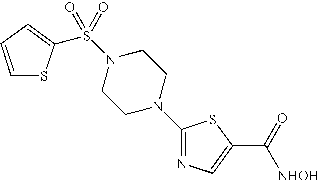

### Preparation of 2-[4-(thiophene-2-benzenesulfonyl)-piperazin-1-Yl]-thiazole-5-carboxylic acid hydroxyamide

HPLC: 58.9% (Rt=12.55 min).

¹H NMR (CD₃OD, 200 MHz): δ 8.67 (1H, m), 7.85 (1H, m), 7.78 (1H, s), 7.59 (1H, m), 7.20 (1H, m), 3.67 (4H, m), 3.17 (4H, m).

MS (m/z): 375 (M+1).

### Example 9

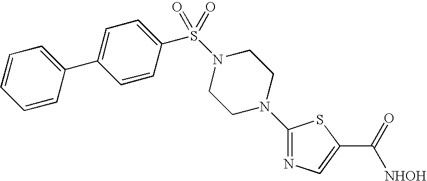

### 2-[4-(biphenyl-4-sulfonyl)-piperazin-1-yl]-thiazole-5-carboxylic acid hydroxyamide

HPLC: 97.51% (Rt=14.61 min).

¹H NMR (CD₃OD, 200 MHz): δ 7.91 (1H, s), 7.76-7.41 (9H, m), 3.69 (4H, m), 3.21 (4H, m).

MS (m/z): 445 (M+1).

### Example 10

### 2-[4-(5-dimethylamino-naphthalene-1-sulfonyl)-piperazin-1-yl]-thiazole-5-carboxylic acid hydroxyamide

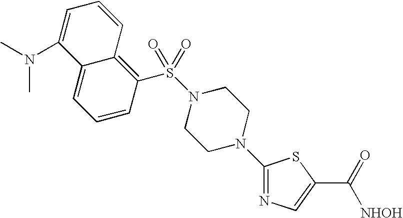

HPLC: 90.69% (Rt=12.77 min).

¹H NMR (CD₃OD, 200 MHz): δ 8.12 (2H, m), 8.33 (1H, m), 7.86-7.59 (4H, m), 3.62 (4H, m), 3.36 (4H, m), 3.16 (6H, s).

MS (m/z): 462 (M+1).

### Example 11

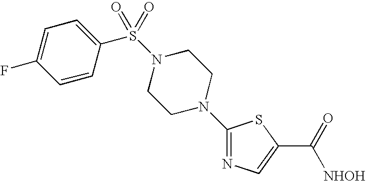

### 2-[4-(4-fluoro-benzenesulfonyl)-piperazin-1-yl]-thiazole-5-carboxylic acid hydroxyamide

MS (m/z): 387 (M+1).

### Example 91

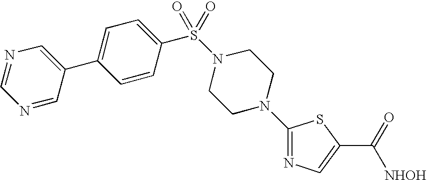

### 2-[4-{(4-pyrimidine)benzenesulfonyl}-piperazin-1-yl]-thiazole-5-carboxylic acid hydroxyamide

HPLC: 94.58% (Rt=11.98 min);

¹H NMR (CD₃OD, 200 MHz): δ 9.22 (1H, s), 9.16 (2H, s), 8.01 (4H, s), 7.75 (1H, bs), 3.69 (4H, m) 3.24 (4H, m);

MS (m/z) 447 (M+1).

### Example 92

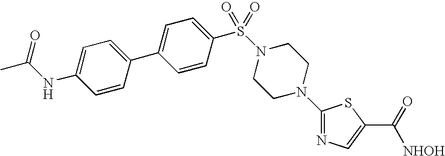

### 2-[4-{(4-acetamidophenyl)benzenesulfonyl{-piperazine-1-yl-thiazole-5-carboxylic acid hydroxyamide

HPLC: 90.18% (Rt=12.90 min);

¹H NMR (CD₃OD, 200 MHz) δ 7.88 (4H, bs), 7.70 (5H, bs), 3.68 (4H, m), 3.22 (4H, m), 2.16 (3H, s);

MS (m/z) 502 (M+1).

### Example 93

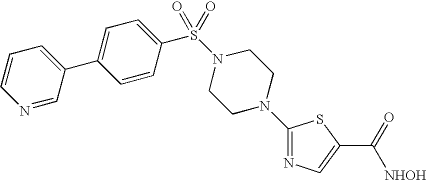

### 2-[4-{(3-pyrido)benzenesulfonyl}-piperazin-1-yl]-thiazole-5-carboxylic acid hydroxyamide

HPLC: 93.06% (Rt=11.23 min);

¹H NMR (CD₃OD, 200 MHz) δ 8.93 (1H, s), 8.65 (1H, d, J=4.8 Hz), 8.22 (1H, m), 7.98 (4H, bs), 7.73 (1H, m), 7.61 (1H, m), 3.71 (4H, m), 3.24 (4H, m); MS (m/z) 446 (M+1).

### Example 94

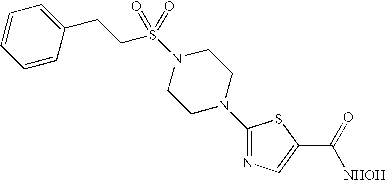

### 2-[4-(phenethylsulfonyl)-piperazin-1-yl]-thiazole-5-carboxylic acid hydroxyamide

HPLC: 97.63% (Rt=13.41 min);

¹H NMR (CD₃OD, 200 MHz) δ 7.81 (1H, bs), 7.31 (5H, m), 3.74 (4H, m), 3.53 (4H, m), 3.45 (2H, m), 3.13 (2H, m);

MS (m/z) 397 (M+1).

### Example 95

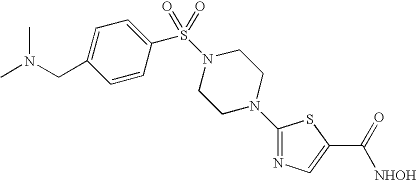

### 2-[4-(4-N,N-dimethylbenzenesulfonyl)-piperazin-1-yl]-thiazole-5-carboxylic acid hydroxyamide

HPLC: 71.37 (Rt=11.17 min);

¹H NMR (CD₃OD, 200 MHz) δ 8.00-7.79 (5H, m), 4.43 (2H, s), 3.81 (4H, m), 3.37 (4H, m), 2.95 (6H, s); MS (m/z) 426 (M+1).

### Example 96

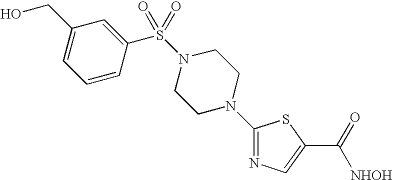

### 2-[4-(4-hydroxymethylbenzenesulfonyl)-piperazin-1-yl]-thiazole-5-carboxylic acid hydroxyamide

HPLC: 84.57% (Rt=11.41 min);

¹H NMR (CD₃OD, 200 MHz) δ 7.82 (1H, bs), 7.66 (4H, m), 4.72 (2H, s), 3.66 (4H, m), 3.16 (4H, m); MS (m/z) 399 (M+1).

### Example 97

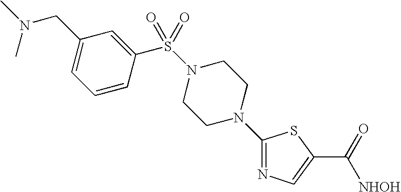

### 2-[4-(3-N,N-dimethylaminobenzenesulfonyl)-piperazin-1-yl]-thiazole-5-carboxylic acid hydroxyamide

HPLC: 80.24% (Rt 10.91 min);

¹H NMR (CD₃OD, 200 MHz) δ 7.93-7.71 (5H, m), 4.08 (2H, s), 3.68 (4H, m), 3.18 (4H, m), 2.62 (6H, s);

MS (m/z) 426 (M⁺, 100.00).

### Example 98

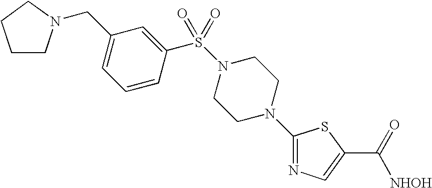

### 2-[4-(3-pyrrolidonobenzenesulfonyl)-piperazin-1-yl]-thiazole-5-carboxylic acid hydroxyamide

HPLC: 99.21% (Rt=11.13 min);

¹H NMR (CD₃OD, 200 MHz) δ 7.82-7.60 (5H, m), 3.76 (2H, s), 3.65 (4H, m), 3.16 (4H, m), 2.58 (4H, m), 1.83 (4H, m);

MS (m/z) 452 (M+1).

### Example 99

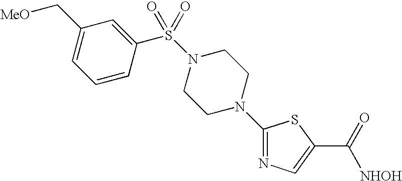

### 2-[4-(3-methoxymethylbenzenesulfonyl)-piperazin-1-yl]-thiazole-5-carboxylic acid hydroxyamide

HPLC: 93.25% (Rt=12.71 min);

¹H NMR (CD₃OD, 200 MHz) δ 7.79-7.60 (5H, m), 4.57 (2H, s), 3.56 (4H, m), 3.44 (3H, s), 3.15 (4H, m);

MS (m/z) 413 (M+1).

### Example 100

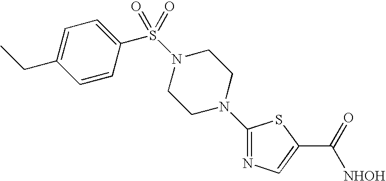

### 2-[4-(4-Ethylbenzenesulfonyl)piperazin-1-yl]-thiazole-5-carboxylic acid hydroxyamide

HPLC: 96.32% (R=14.02 min);

¹HNMR (CD₃OD, 200 MHz) δ 7.73 (3H, m), 7.48 (2H, m), 3.66 (4H, m), 3.14 (4H, m), 2.77 (2H, q, J=7.4 Hz), 1.28 (3H, t, J=7.5 Hz);

MS (m/z): 396 (M+1)

### Example 101

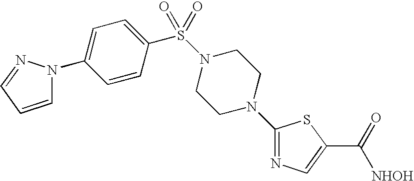

### 2-[4-(4-pyrazolylbenzenesulfonyl)-piperazin-1-yl]-thiazole-5-carboxylic acid hydroxyamide

HPLC: 93.63% (Rt=12.87 min);

¹HNMR (CD₃OD, 200 MHz) δ 8.20 (1H, s), 8.13 (2H, d, J=8.5 Hz), 7.91 (2H, d, J=8.5 Hz), 7.81 (1H, s), 7.52 (1H, m), 6.64 (1H, s), 3.79 (4H, m), 3.27 (4H, m);

MS (m/z) 435 (M+1).

### Example 102

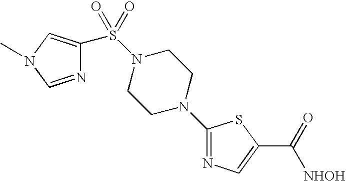

### 2-[4-(1-Methyl-4,5-dihydroimidazolulfonyl)piperazin-1-yl]-thiazole-5-carboxylic acid hydroxyamide

HPLC: 96.97% (Rt=7.69 min);

¹H NMR (CD₃OD, 200 MHz) δ 8.74 (1H, s), 8.16 (1H, s), 7.78 (1H, bs), 3.95 (3H, s), 3.87 (4H, m), 3.42 (4H, m);

MS (m/z) 373 (M+1).

### Example 103

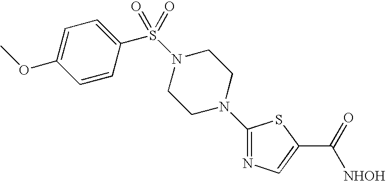

### 2-[4-(4-Methoxybenzenesulfonyl)piperazin-1-yl]-thiazole-5-carboxylic acid hydroxyamide

HPLC 88.16% (Rt=14.96 min);

¹H NMR (CD₃OD, 200 MHz) δ 7.79 (3H, m), 7.17 (2H, d, J=9.2 Hz), 3.90 (3H, s), 3.76 (4H, m), 3.28 (4H, m);

MS (m/z) 399 (M+1).

### Example 104

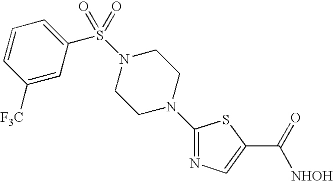

### 2-[4-(4-Trifluoromethylbenzenesulfonyl)-piperazin-1-yl]-thiazole-5-carboxylic acid hydroxyamide

HPLC: 95.02% (Rt=16.12 min);

¹H NMR (CD₃OD, 200 MHz) δ 8.11 (3H, m), 7.92 (2H, m), 3.78 (4H, m), 3.23 (4H, m);

MS (m/z) 437 (M+1).

### Example 105

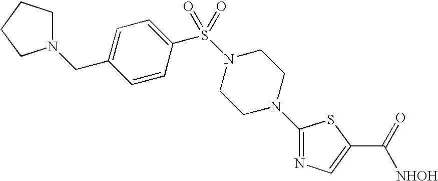

### 2-[4-(4-Pyrrolidinobenzenesulfonyl)piperazin-1-yl]-thiazole-5-carboxylic acid hydroxyamide

HPLC: 91.98% (Rt=11.05 min);

¹H NMR (CD₃OD, 200 MHz) δ 7.97-7.82 (5H, m), 4.52 (2H, s), 3.83-3.77 (4H, m), 3.60 (2H, m), 3.34-3.18 (6H, m), 2.22-2.05 (4H, m);

MS (m/z) 451 (M+1).

### Example 106

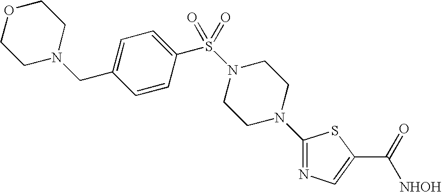

### 2-[4-(4-Morpholinibenzenesulfonyl)-piperazin-1-yl]-thiazole-5-carboxylic acid hydroxyamide

HPLC: 91.80% (RT 10.89 min);

¹H NMR (CD₃OD, 200 MHz) δ 7.95 (3H, m), 7.85 (2H, d, J=7.2 Hz), 4.51 (2H, s), 4.05 (2H, m), 3.87-3.71 (6H, m), 3.49-3.22 (8H, m);

MS (m/z) 468 (M+1).

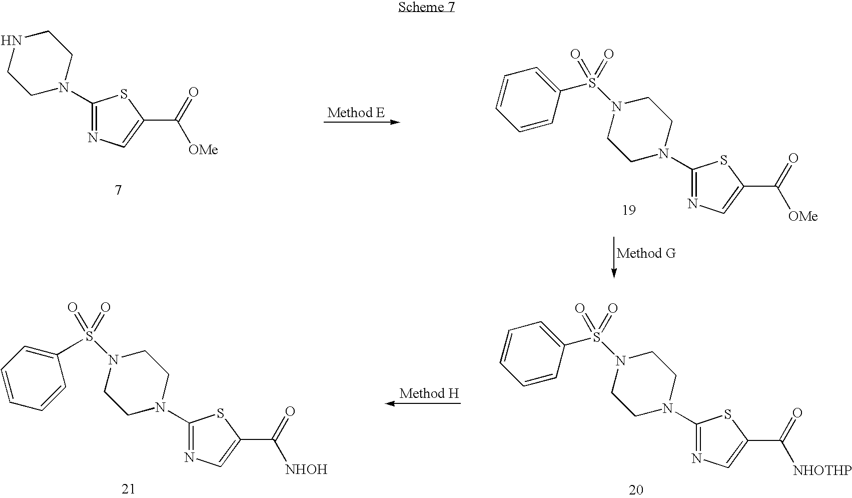

### Example 12

### 2-[4-(benzenesulfonyl)-piperazin-1-yl]-thiazole-5-carboxylic acid hydroxyamide

Method E: To a solution of compound (7) (150 mg, 0.660 mmol) in dichloromethane (7.5 mL) were added benzenesulfonyl chloride (133 mg, 0.750 mmol) and triethylamine (146 mg, 2.169 mml) under N₂ atmosphere. The reaction mixture was stirred at room temperature for 4 h (progress of the reaction was monitored by TLC analysis). Water (10 mL) followed by dichloromethane (10 mL) were added to the reaction mixture and the organic layer was separated, dried on sodium sulphate, filtered and concentrated under reduced pressure. The residue was purified by column chromatography using silica gel to give the compound 19 as a solid (190 mg).

HPLC: 96.50% (Rt=15.13 min). ¹H NMR (CDCl₃, 200 MHz) δ 7.82 (1H, s), 7.80-7.56 (5H, m), 3.81 (3H, s), 3.68 (4H, t, J=5.2 Hz), 3.14 (4H, t, J=5.2 Hz).

Method G: To a solution of compound (19) (200 mg, 0.545 mmol) in methanol (5 mL) was added a solution of sodium hydroxide (152 mg, 3.81 mmol) in water (5 mL) and the reaction mixture was stirred at room temperature for 5 h. The reaction mixture pH was adjusted to neutral by adding 1N HCl and methanol was evaporated on a rotavap. The compound was extracted thrice with ethyl acetate and the combined ethyl acetate layers were dried over sodium sulfate, filtered, concentrated and dried under vacuum for 1 h to give the corresponding acid. To the isolated acid (160 mg, 0.453 mmol) in DCM (15 mL) were added EDC (173 mg, 0.907 mmol), HOBt (91 mg, 0.679 mmol), DIEA (112 mg, 0.907 mmol) and NH₂OTHP (51 mg, 0.430 mmol) simultaneously under nitrogen atmosphere and the reaction mixture was stirred at room temperature for 12 h. The reaction mixture was quenched with water and the compound was extracted thrice with DCM and the combined DCM layers were dried over sodium sulfate, filtered, concentrated and purified over silica gel to give compound 20 (93 mg).

HPLC: 92.62% (Rt=14.17 min).

Method H: To a solution of compound 20 (93 mg, 0.205 mmol) in MeOH (2 mL) were added freshly prepared 20% solution of HCl in ether (10 mL) at 0° C., and the reaction mixture was stirred at the same temperature for 20 min (progress of the reaction was monitored by HPLC analysis). After complete disappearance of the starting material, solvent was evaporated from the reaction mixture under reduced pressure and the residue was completely dried on high vaccum pump. To the residue was added chilled ether (10 mL) to obtain compound 21 as a white solid (58 mg).

HPLC: 98.72% (Rt 12.523 min).

Following the procedures set forth in Example 12 above, the compounds of following examples were prepared using the appropriate starting materials and the ¹H NMR data, HPLC and/or mass spectral data are presented below.

### Example 13

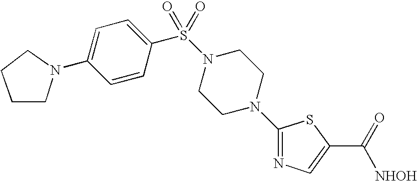

### 2-[4-(4-pyrolidinylbenzenesulfonyl)-piperazin-1-yl]-thiazole-5-carboxylic acid hydroxyamide

HPLC: 98.72% (RT 12.523 min);

MS m/e: 438 (M+1).

### Example 14

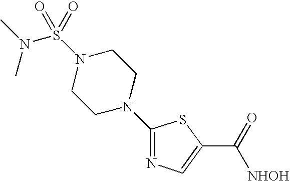

### 2-[4-(N,N-dimethylsulfonyl)-piperazin-1-yl]-thiazole-5-carboxylic acid hydroxyamide

HPLC: 95% (Rt=10.81 min).

¹H NMR (CD₃OD, 200 MHz) δ 7.92 (1H, s), 3.77 (4H, t, J=4.8 Hz), 3.48 (4H, t, J=4.8 Hz), 2.88 (6H, s);

MS m/e: 336 (M+1).

### Example 15

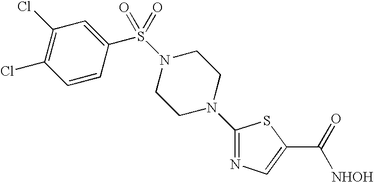

### 2-[4-(3,4-dichlorolphenylsulfonyl)-piperazin-1-yl]-thiazole-5-carboxylic acid hydroxyamide

HPLC: 94.85% (Rt=14.24 min);

MS m/e: 436 (M+1).

### Example 16

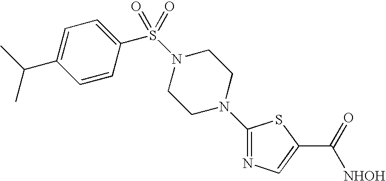

### 2-[4-(4-isopropylphenylsulfonyl)-piperazin-1-yl]-thiazole-5-carboxylic acid hydroxyamide

HPLC: 95.14% (Rt=14.20 min);

MS m/e: 411 (M+1).

### Example 17

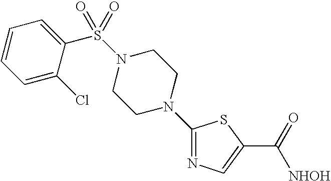

### 2-[4-(2-chlorophenalsulfonyl)-piperazin-1-yl]-thiazole-5-carboxylic acid hydroxyamide

HPLC: 95.66% (Rt=13.01 min).

¹H NMR (CD₃OD, 200 MHz): δ 8.13-7.52 (m, 5H), 3.71 (4H, t, J=5.4 Hz), 3.51 (4H, t, J=5.4 Hz); MS m/e: 402 (M+1).

### Example 18

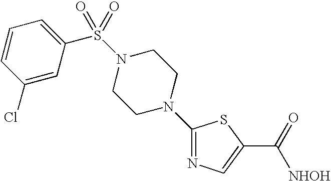

### 2-[4-(3-chlorophenylsulfonyl)-piperazin-1-yl]-thiazole-5-carboxylic acid hydroxyamide

HPLC: 96.91% (Rt=13.45 min);

MS m/e: 403 (M+1).

### Example 19

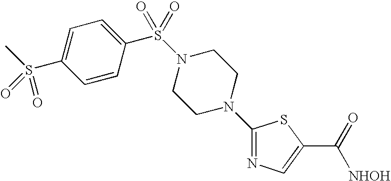

### 2-[4-(4-methylsufonylphenylsulfonyl)-piperazin-1-yl]-thiazole-5-carboxylic acid hydroxyamide

HPLC: 93.09% (Rt=12.02 min).

¹H NMR (CD₃OD, 200 MHz): δ 8.25 (2H, d, J=8.4 Hz), 8.11 (2H, d, J=8.4 Hz), 7.81 (1H, bs), 3.77 (4H, m), 3.22 (4H, m), 3.21 (3H, s);

MS m/e: 447 (M+1).

### Example 20

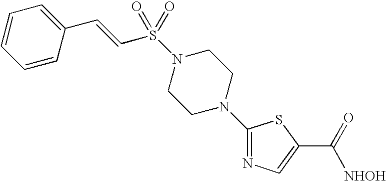

### 2-[4-(trans-2-phenylethanelsulfonyl)piperazin-1-yl]-thiazole-5-carboxylic acid hydroxyamide

HPLC: 96.66% (Rt=13.44 min).

MS m/e: 395 (M+1).

### Example 21

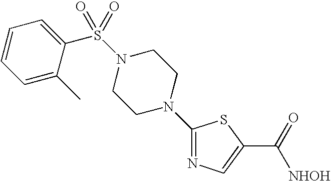

### 2-[4-(2-methylphenylsulfonyl)piperazin-1-yl]-thiazole-5-carboxylic acid hydroxyamide

HPLC: 90.21% (Rt=13.14 min).

¹H NMR (CD₃OD, 200 MHz): δ 7.92-7.31 (5H, m), 3.77 (4H, t, J=4.8 Hz), 3.48 (4H, t, J=4.8 Hz), 2.88 (3H, s);

MS m/e: 383 (M+1).

### Example 22

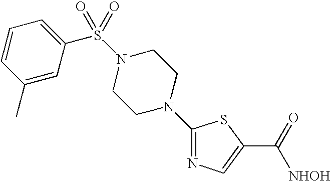

### 2-[4-(3-methylphenylsulfonyl)-piperazin-1-yl]-thiazole-5-carboxylic acid hydroxyamide

HPLC: 96.27% (Rt=13.17 min);

¹H NMR (CD₃OD, 200 MHz): δ 7.92-7.31 (5H, m), 3.77 (4H, t, J=4.8 Hz), 3.48 (4H, t, J=4.8 Hz), 2.88 (3H, s);

MS (m/z) 383 (M+1).

### Example 23

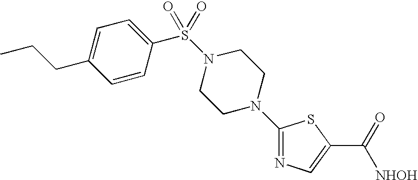

### 2-[4-(4-n-propylphenylsulfonyl)-piperazin-1-yl]-thiazole-5-carboxylic acid hydroxyamide

HPLC: 95.53% (Rt=14.35 min);

¹H NMR (CD₃OD, 200 MHz): δ 7.77 (3H, m), 7.50 (2H, d, J=8.4 Hz) 3.73 (4H, t, J=4.6 Hz), 3.23 (4H, t, J=4.6 Hz), 2.72 (2H, t, J=7.6 Hz), 1.68 (2H, m), 0.98 (2H, t, J=7.2 Hz);

MS (m/z) 411 (M+1).

### Example 24

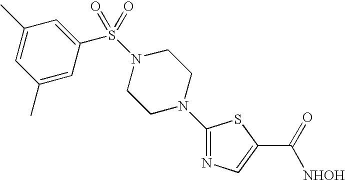

### 2-[4-(3,5-dimethylphenylsulfonyl)-piperazin-1-yl]-thiazole-5-carboxylic acid hydroxyamide

HPLC: 94.72% (Rt=13.71 min);

¹H NMR (CD₃OD, 200M Hz) δ 7.92-7.37 (4H, m), 3.77 (4H, t, J=4.8 Hz), 3.23 (4H, t, J=4.8 Hz), 2.42 (6H, s);

MS (m/z) 397 (M+1).

### Example 25

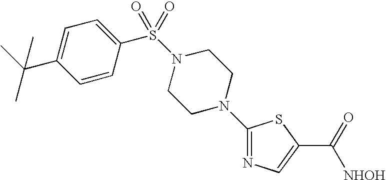

### 2-[4-(4-t-butylphenylsulfonyl)-piperazin-1-yl]-thiazole-5-carboxylic acid hydroxyamide

HPLC: 95.73% (Rt=14.62 min);

¹H NMR (CD₃OD, 200 MHz): δ 7.79-7.67 (5H, m), 3.76 (4H, m), 3.25 (4H, m), 1.34 (9H, s);

MS (m/z) 425 (M+1).

### Example 26

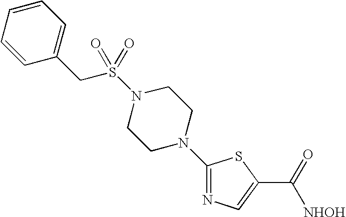

### 2-[4-(benzylsulfonyl)piperazin-1-yl]-thiazole-5-carboxylic acid hydroxyamide

HPLC: 98.36% (Rt=12.72 min);

¹HNMR (CD₃OD, 200 MHz) δ 7.83 (1H, m), 7.46-7.39 (5H, m), 4.46 (2H, s), 3.64 (4H, t, J=4.8 Hz), 3.34 (4H, t, J=4.8 Hz);

MS (m/z) 383 (M+1).

### Example 27

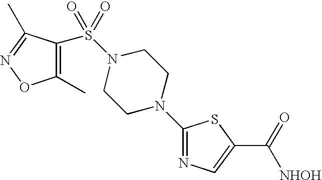

### 2-[4-(3,5-dimethylisoxazolesulfonyl)-pipiperazin-1-yl]-thiazole-5-carboxylic acid hydroxyamide

HPLC: 96.54% (Rt=12.48 min);

¹H NMR (CD₃OD, 200 MHz): δ 7.83 (1H, m), 3.78 (4H, bt), 3.37 (4H, bt), 2.69 (3H, s), 2.42 (3H, s);

MS (m/z) 388 (M+1).

### Example 28

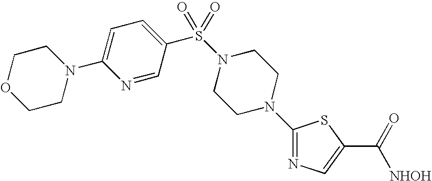

### 2-[4-({4-morpholino}-3-pyridylsulfonyl)piperazin-1-yl]-thiazole-5-carboxylic acid hydroxyamide

HPLC: 90.72% (Rt=11.98 min);

¹H NMR (CD₃OD, 200 MHz): δ 8.50 (1H, bs), 8.20-7.20 (4H, m), 3.83 (12H, m), 3.33 (4H, m);

MS (m/z) 455 (M+1).

### Example 29

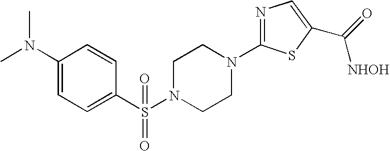

### 2-[4-(4-dimethylaminophenylsulfonyl)-piperazin-1-yl]-thiazole-5-carboxylic acid hydroxyamide

¹H NMR (DMSO-d₆, 400 MHz): δ 7.8 (bs, 1H, Ar—H), 7.5 (d, J=8 Hz, 2H, Ar—H), 6.81 (d, J=8 Hz, 2H, Ar—H), 3.54 (t, J=4 Hz, 4H, 2CH₂), 3.01 (s, 6H, N(Me)₂), 2.92 (t, J=4 Hz, 2CH₂).

MS (m/z)=412 (M+1).

### Example 30

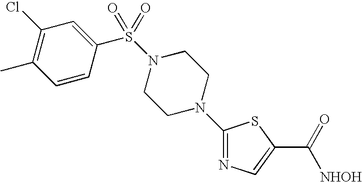

### 2-[4-(3-chloro-4-methylphenylsulfonyl)-piperazin-1-yl]-thiazole-5-carboxylic acid hydroxyamide

HPLC: 92% (Rt=14.31 min).

¹H NMR (CD₃OD, 200 MHz) δ 7.81 (2H, m), 7.62 (2H, m), 3.81 (4H, m), 3.32 (4H, m), 2.47 (3H, s);

MS m/e: 417 (M+1).

### Example 31

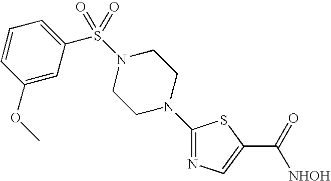

### 2-[4-(4-methoxyphenylsulfonyl)-piperazin-1-yl]-thiazole-5-carboxylic acid hydroxyamide

HPLC: 91% (Rt=13.37 min).

¹H NMR (CD₃OD, 200 MHz) δ 7.55 (2H, m), 7.33 (3H, m), 3.89 (3H, s), 3.60 (4H, m), 3.32 (4H, m);

MS m/e: 399 (M+1).

### Example 32

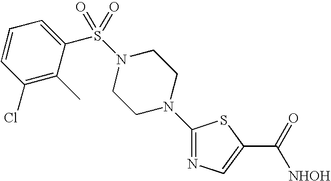

### 2-[4-(3-chloro-2-methylphenylsulfonyl)-piperazin-1-yl]-thiazole-5-carboxylic acid hydroxyamide

HPLC: 97.4% (Rt=14.45 min).

¹H NMR (CD₃OD, 200 MHz): δ 7.94 (2H, m), 7.75 (1H, m), 7.45 (1H, m), 3.78 (4H, m), 3.48 (4H, m), 2.71 (3H, s);

MS m/e: 417 (M+1).

### Example 33

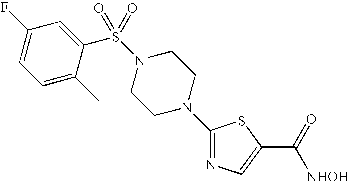

### 2-[4-(2-methyl-5-fluorophenylsulfonyl)-piperazin-1-yl]-thiazole-5-carboxylic acid hydroxyamide

HPLC: 97.5% (Rt=13.86 min).

¹H NMR (CD₃OD, 200 MHz): δ 7.70 (1H, d, J=2.6 Hz), 7.66 (1H, d, J=2.6 Hz), 7.48 (1H, m), 7.35 (1H, m), 3.81 (4H, m), 3.49 (4H, m), 2.62 (3H, s);

MS m/e: 401 (M+1).

### Example 34

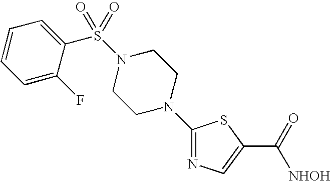

### 2-[4-(2-fluorophenylsulfonyl)-piperazin-1-yl]-thiazole-5-carboxylic acid hydroxyamide

HPLC: 95% (Rt=13.06 min).

¹H NMR (CD₃OD, 200 MHz): δ 7.90 (2H, m), 7.77 (1H, m), 7.43 (2H, m), 3.78 (4H, m), 3.39 (4H,m);

MS m/e: 387 (M+1).

### Example 35

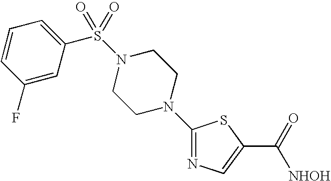

### 2-[4-(3-fluorophenylsulfonyl)-piperazin-1-yl]-thiazole-5-carboxylic acid hydroxyamide

HPLC: 90% (Rt=13.21 min).

¹H NMR (CD₃OD, 200 MHz): δ 7.92 (1H, s), 7.65 (2H, m), 7.51 (2H, m), 3.82 (4H, m), 3.34 (4H, m);

MS m/e: 387 (M+1).

### Example 36

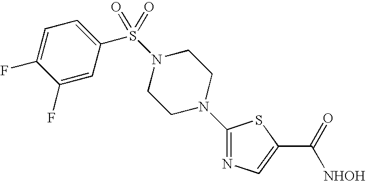

### 2-[4-(3,4-difluorophenylsulfonyl)-piperazin-1-yl]-thiazole-5-carboxylic acid hydroxyamide

HPLC: 82% (Rt=13.71 min).

¹H NMR (CD₃OD, 200 MHz): δ 7.83 (2H, m), 7.63 (2H, m), 3.84 (4H, m), 3.33 (4H,m);

MS m/e: 405 (M+1).

### Example 37

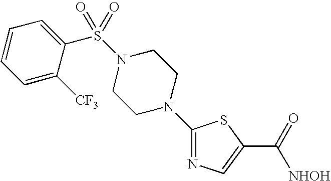

### 2-[4-(2-trifluoromethylphenylsulfonyl)-piperazin-1-yl]-thiazole-5-carboxylic acid hydroxyamide

HPLC: 95.03% (Rt=13.94).

¹H NMR (CD₃OD, 200 MHz): δ 8.21 (1H, m), 8.04 (1H, m), 7.90 (3H, m), 3.81 (4H, m), 3.53 (4H,m);

MS m/e: 437 (M+1).

### Example 38

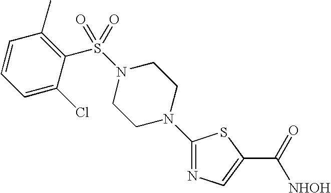

### 2-[4-(2-methyl-6-chlorophenylsulfonyl)piperazin-1-yl-thiazole-5-carboxylic acid hydroxyamide

HPLC: 90% (Rt=13.89 min).

¹H NMR (CD₃OD, 200 MHz): δ 7.93 (1H, s), 7.47 (2H, m), 7.40 (1H, m), 3.66 (4H, m), 3.32 (4H, m), 2.74 (3H, s);

MS m/e: 417 (M+1).

### Example 39

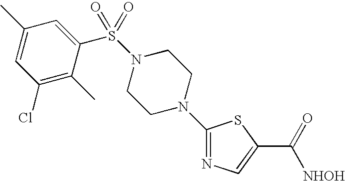

### 2-[4-(2,5-dimethyl-4-chlorophenylsulfonyl)-piperazin-1-yl]-thiazole-5-carboxylic acid hydroxyamide

HPLC: 96.3% (Rt=15.01 min).

¹H NMR (CD₃OD, 200 MHz): δ 7.85 (2H, m), 7.48 (1H, m), 3.76 (4H, m), 3.42 (4H, m), 2.59 (3H, s) 2.43 (3H, s);

MS m/e: 431 (M+1).

### Example 40

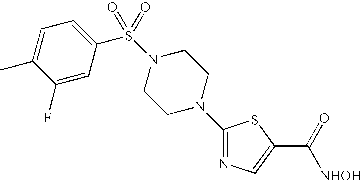

### 2-[4-(3-fluoro-4-methylphenylsulfonyl)-piperazin-1-yl]-thiazole-5-carboxylic acid hydroxyamide

HPLC: 93% (Rt=13.90 min).

¹H NMR (CD₃OD, 200 MHz): δ 7.90 (1H, s), 7.53 (3H, m), 3.77 (4H, m), 3.30 (4H, m), 2.37 (3H, s);

MS m/e: 401 (M+1).

### Example 41

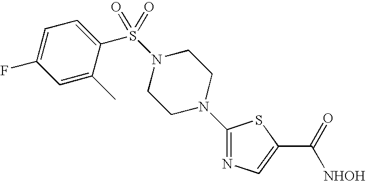

### 2-[4-(4-fluoro-2-methylphenylsulfonyl)-piperazin-1-yl]-thiazole-5-carboxylic acid hydroxyamide

HPLC: 97.5% (Rt=13.88 min).

¹H NMR (CD₃OD, 200 MHz) δ 8.01 (1H, m), 7.92 (1H, s), 7.20 (2H, m), 3.71 (4H, m), 3.43 (4H, m), 2.66 (3H, s);

MS m/e: 401 (M+1).

### Example 42

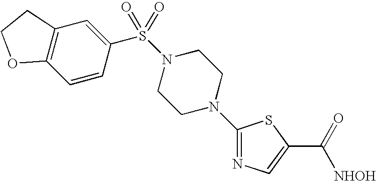

### 2-[4-(2,3-dihydrobenzofuransulfonyl)-piperazin-1-yl]-thiazole-5-carboxylic acid hydroxyamide

HPLC: 93.2% (Rt=13.14 min).

¹H NMR (CD₃OD, 200 MHz): δ 7.67 (1H, s), 7.63 (1H, d, J=2.2 Hz), 7.59 (1H, d, J=2.2 Hz), 4.69 (2H, t, J=8.8 Hz)), 3.81 (4H, m), 3.34 (2H, m), 3.24 (4H, m);

MS m/e: 411 (M+1).

### Example 43

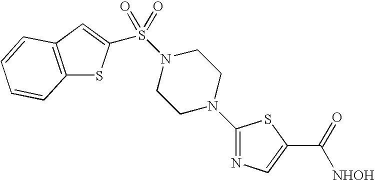

### 2-[4-(2-benzothiphenesulfonyl)-piperazin-1-yl-thiazole-5-carboxylic acid hydroxyamide

HPLC: 93% (Rt=14.30 min).

¹H NMR (CD₃OD, 200 MHz): δ 8.01 (4H, m), 7.54 (2H, m), 3.79 (4H, m), 3.44 (4H, m).

MS m/e: 425 (M+1).

### Example 44

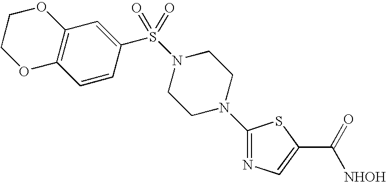

### 2-[4-(3,4-benzodioxansulfonyl)-piperazin-1-yl]-thiazole-5-carboxylic acid hydroxyamide

HPLC: 98.8% (Rt=13.14 min).

¹H NMR (CD₃OD, 200 MHz): δ 7.30 (2H, m), 7.06 (2H, m), 4.33 (4H, m), 3.75 (4H, m), 3.34 (4H, m);

MS m/e: 427 (M+1).

### Example 45

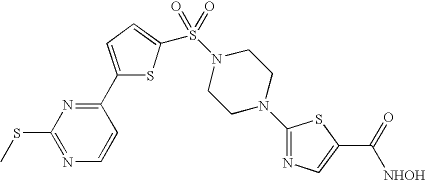

### 2-[4-(2-{2-methylthiopyrimidine-4-yl}-5-thiophenesulfonyl)-piperazin-1-yl]-thiazole-5-carboxylic acid hydroxyamide

HPLC: 96.3% (Rt=14.31 min).

¹H NMR (CD₃OD, 200 MHz): δ 8.60 (1H, d, J=5.2 Hz), 8.01 (1H, d, J=4.4 Hz), 7.68 (1H, m), 7.62 (1H, s), 7.59 (1H, s), 3.73 (4H, m), 3.31 (4H, m), 2.62 (3H, s);

MS m/e: 499 (M+1).

### Example 46

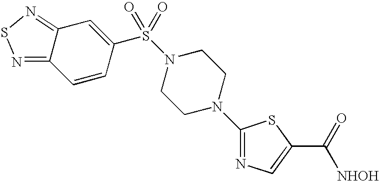

### 2-[4-(2,1,3-benzothiadiazole-5-sulfonyl)-piperazin-1-yl]-thiazole-5-carboxylic acid hydroxyamide

HPLC: 90.46% (Rt=16.04 min).

¹H NMR (CD₃OD, 200 MHz): δ 8.59 (1H, m), 8.24 (1H, m), 8.03 (1H, d, J=1.8 Hz), 7.79 (1H, m), 3.72 (4H, m), 3.32 (4H, m);

MS m/e: 427 (M+1).

### Example 47

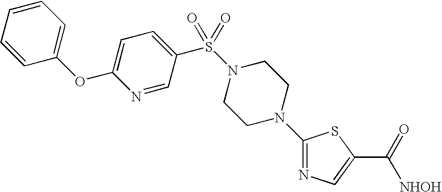

### 2-[4-(2-phenoxypyridine-5-sulfonyl)-piperazin-1-yl]-thiazole-5-carboxylic acid hydroxyamide

HPLC: 92% (Rt=14.27 min).

¹H NMR (CD₃OD, 200 MHz): δ 8.52 (1H, m), 8.19 (1H, m), 8.15 (1H, m), 7.30 (2H, m), 7.15 (2H, m), 3.68 (4H, m), 3.34 (4H, m);

MS m/e: 462 (M+1).

### Example 48

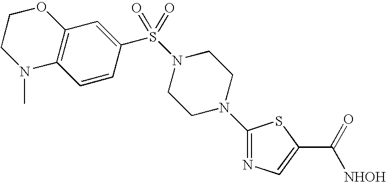

### 2-[4-(N-methyl-2,3-dihydrobenzisoxazinylsulfonyl)-piperazin-1-yl]-thiazole-5-carboxylic acid hydroxyamide

HPLC: 92%.

¹H NMR (CD₃OD, 300 MHz): δ 7.62 (s, 1), 6.99-7.03 (dd, J=1.80, 6.30 Hz, 1H), 6.95 (d, J=2.1 Hz, 1H), 6,84 (d, J=8.40 Hz, 1H), 4.31 (t, J=4.8 Hz, 2H), 3.63 (4H, m), 3.34 (4H,m), 3.10 (t, J=6.0 Hz, 2H), 2.93 (s, 3H);

MS m/e: 440 (M+1).

### Example 49

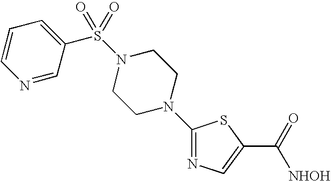

### 2-[4-(pyridine-3-sulfonyl)-piperazin-1-yl]-thiazole-5-carboxylic acid hydroxyamide

¹H NMR (CD₃OD, 300 MHz): δ 8.88 (m, 2H), 8.20 (m, 1H), 7.68 (m, 2H), 3.56 (m, 2H), 3.12 (m, 2H);

MS m/e: 370 (M+1).

### Example 50

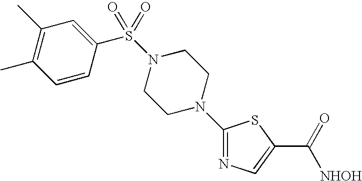

### 2-[4-(3,4-dimethylphenylsulfonyl)-piperazin-1-yl]-thiazole-5-carboxylic acid hydroxyamide

¹H NMR (CD₃OD, 300 MHz): δ 7.74 (s, 1H), 7.53 (m, 2H), 7.37 (m, 1H), 3.63 (m, 4H), 3.10 (m, 4H), 2.36 (s, 6H);

MS m/e: 397 (M+1).

### Example 51

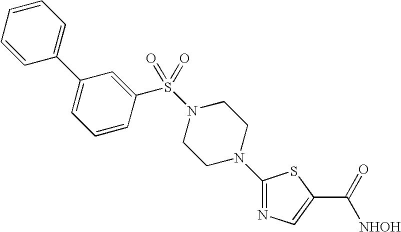

### 2-[4-(3-biphenylsulfonyl)-piperazin-1-yl]-thiazole-5-carboxylic acid hydroxyamide

¹H NMR (CD₃OD, 300 MHz): δ 7.90 (m, 2H), 7.75 (m, 6H), 7.42 (m, 2H), 3.63 (m, 4H), 3.26 (m, 4H).

MS m/e: 445 (M+1).

### Example 52

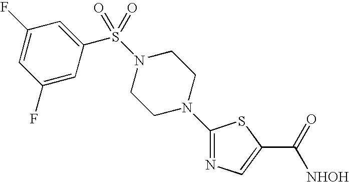

### 2-[4-(3,5-difluorophenylsulfonyl)-piperazin-1-yl]-thiazole-5-carboxylic acid hydroxyamide

HPLC: 91.7% (Rt=13.67 min);

¹H NMR (CD₃OD, 200 MHz) δ 7.8 (1H, hump), 7.46 (2H, m), 7.37 (1H, m), 3.68 (4H, m), 3.22 (4H, m);

MS (m/z) 406 (M+1).

### Example 53

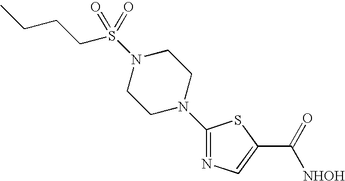

### 2-[4-(n-butylsulfonyl)piperazin-1-yl]-thiazole-5-carboxylic acid hydroxyamide

HPLC: 96% (Rt=12.92 min);

¹H NMR (CD₃OD, 200 MHz): δ 7.76 (1H, s), 3.67 (4H, m), 3.43 (4H, m), 3.07 (2H, t, J=6.8 Hz), 1.78 (2H, m), 1.49 (2H, m), 1.01 (3H, t, J=6.8 Hz);

MS (m/z) 349 (M+1).

### Example 54

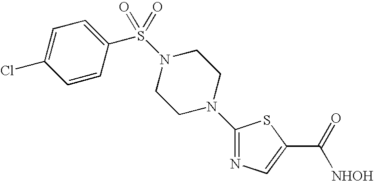

### 2-[4-(chlorophenalsulfonyl)-piperazin-1-yl]-thiazole-5-carboxylic acid hydroxyamide

HPLC: 98% (Rt=14.01 min);

¹H NMR (CD₃OD, 200 MHz): δ 7.79 (2H, m), 7.66 (3H, m), 3.65 (4H, m) 3.19 (4H, m);

MS (m/z) 403 (M+1).

### Example 55

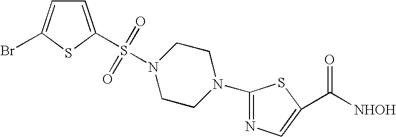

### 2-{4-[(5-bromothien-2-yl)sulfonyl]piperazin-1-yl}-N-hydroxy-1,3-thiazole-5-carboxamide

White solid (2.5 mg), MS m/e 453 (M+H⁺).

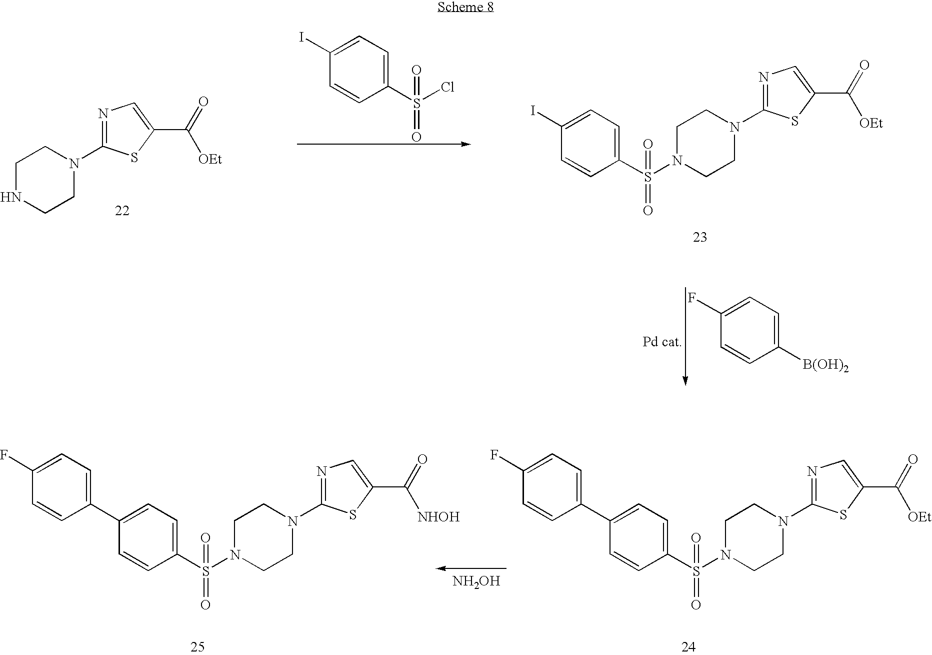

### Example 56

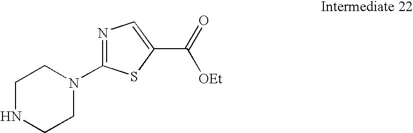

To a solution of piperazine (4.7 g, 0.55 mol) in acetonitrile (60 mL), K₂CO₃ (35 g, 0.25 mol) and ethyl 2-piperazin-1-yl-1,3-thiazole-5-carboxylate (10 g, 42.3 mmol) in acetonitrile (40 m]L) were added and refluxed overnight. The reaction mixture was filtered and concentrated in vacuo. The residue was taken up in CH₂Cl₂ (40 mL) and this layer was washed with water, brine solution, dried over anhydrous Na₂SO₄ and concentrated to afford crude intermediate 22. Crude residue was purified using silica gel column (2% MeOH in CHCl₃) to obtain pure intermediate 22 (5 g, 49%).

R_{f}=0.3;

¹H NMR (400 MHz, CDCl₃) δ=7.86 (s, 1H), 4.28 (q, J=7.1 Hz, J=14 Hz, 2H), 3.52 (t, J=5.0 Hz, 4H), 2.97 (t, J=5.1 Hz, 4H). 1.32 (t, J=7.1 Hz, 3H).

LCMS (electrospray), m/e 242 (C₁₀H₁₅N₃O₂S+H).

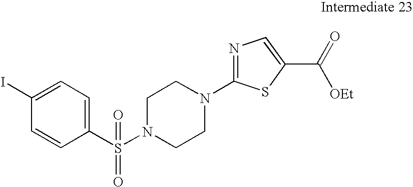

To a solution of intermediate 22 (2.8 g, 11.6 mmol) in CH₂Cl₂ (30 mL) was added triethyl amine (3.2 mL, 23 mmol) and pipsyl chloride (3.9 g, 0.13 mol) over a period of one hour and refluxed for 5 hours. The mixture was diluted with CH₂Cl₂ (50 mL), washed with water, brine solution, dried over anhydrous Na₂SO₄ and concentrated in vacuo. The crude product was purified using silica gel column (1% MeOH in CHCl₃) to obtain pure intermediate 23 (4.1 g, 69%).

R_{f}=0.8; ¹H NMR (400 MHz, CDCl₃)

δ=7.93 (d, J=7.8 Hz, 2H), 7.48 (d, J=7.8 Hz, 2H), 4.31 (q, J=6.7 Hz, 2H), 3.71 (t, J=4.8 Hz, 4H), 3.16 (t, J=4.8 Hz, 4H), 1.35 (t, J=6.7 Hz, 3H).

LCMS (electrospray), m/e 508 (C₁₆H₁₈IN₃O₄S₂+H).

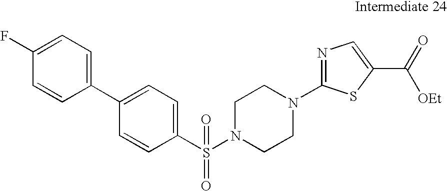

To a solution of intermediate 23 (0.5 g, 0.99 mmol) in DMF (15 mL, 4-fluorophenylphenylboronic acid (0.276 g, 1.97 mmol), palladium acetate (0.066 g, 0.296 mmol), triphenyl phosphine (0.078 g, 0.296 mmol) and cesium carbonate (0.964 g, 2.96 mmol) were added under nitrogen atmosphere and heated at 80° C. overnight. The crude material was as such used for column purification (50% EtOAc in petroleum ether) to afford intermediate 24 (0.54 g, 57%).

R_{f}=0.6;

¹H NMR (400 MHz, CDCl₃) δ=7.85-7.56 (m, 7H), 7.22-7.18 (m, 2H), 4.29 (q, J=7.1 Hz, 2H), 3.73 (t, J=5.0 Hz, 4H), 3.22 (t, J=5.0 Hz, 4H), 1.31 (t, J=7.1 Hz, 3H);

LCMS (electrospray), mile 476 (M⁺+1) (C₂₂H₂₂FN₃O₄S₂+H)

### Example 56

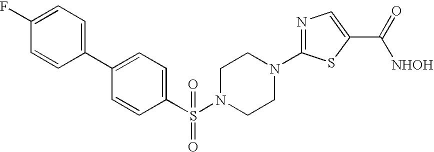

### 2-{4-[(4′-fluoro-1,1′-biphenyl-4-yl)sulfonyl]piperazin-1-yl}-1,3-thiazole-5-carboxylic acid hydroxyamide

[Alternative nomenclature: N-hydroxy-2-{4-[(4′-fluoro-1,1′-biphenyl-4-yl)sulfonyl]piperazin-1-yl}-1-1,3-thiazole-5-carboxamide]

To a stirred solution of Suzuki coupled intermediate 24 (0.5 g, 0.315 moles) in dioxane (7.0 mL) at 0° C. was added hydroxylamine (50% solution in water, 0.2 mL, 3.15 mmol) and the resulting mixture was stirred for 1.0 h at 0° C. The reaction mixture was brought to rt and stirred overnight. After removal of dioxane, water (2.0 mL) was added and acidified with 1.5 N HCl. The mixture was concentrated and subjected to preparative HPLC purification to get corresponding hydroxamic acid 14.0 mg (11%).

¹H NMR (400 MHz, DMSO-d₆) δ=7.95-7.8 (m, 6H, 6Ar—H), 7.36 (t, 3H, 3Ar—H), 3.67 (d, 4H, 2CH₂), 3.1 (d, 4H, 2CH₂).

LCMS (electrospray), m/e 462.8 (M⁺) (M, 462.5 Calcd for C₂₀H₁₉FN₄O₄S₂)

Following the procedures set forth in Example 56 above, the compounds of following Examples 57-72 were prepared according to Scheme 8 were prepared using the appropriate starting materials and the ¹H NMR data, HPLC and/or mass spectral data are presented below.

### Example 57

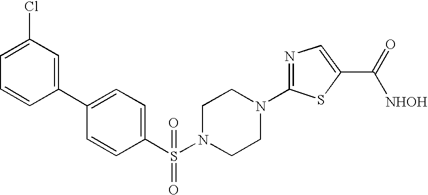

### 2-{4-[(3′-chloro-1,1′-biphenyl-4-yl)sulfonyl]piperazin-1-yl}-1,3-thiazole-5-carboxylic acid hydroxyamide

Yield (35 mg, 12%)

¹H NMR (DMSO-d₆, 400 MHz) δ=7.99 (d, J=8 Hz, 2H, Ar—H), 7.84 (d, J=8 Hz, 2H, Ar—H), 3.55 (d, 4H, 2CH₂), 3.04 (d, 4H, 2CH₂).

LCMS electrospray), m/e 478.8 (M⁺) (M, 478.9 Calcd for C₂₀H₁₉ClN₄O₄S₂)

### Example 58

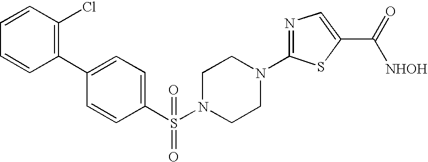

### 2-{4-[(2′-chloro-1,1′-biphenyl-4-yl)sulfonyl]piperazin-1-yl}-1,3-thiazole-5-carboxylic acid hydroxyamide

Yield (35 mg, 12%)

¹H NMR (DMSO-d₆, 400 MHz) δ=7.86 (d, J=8 Hz, 2H, Ar—H), 7.73 (d, J=8.3 Hz, 2H, 2Ar—H), 7.64-7.22 (m, 5H, 5Ar—H), 3.6 (t, J=4.7 Hz, 4H, 2CH₂), 3.1 (t, J=4.8 Hz, 2CH₂).

LCMS (electrospray), m/e 478.8 (M⁺) (M, 478.9 Calcd for C₂₀H₁₉ClN₄O₄S₂).

### Example 59

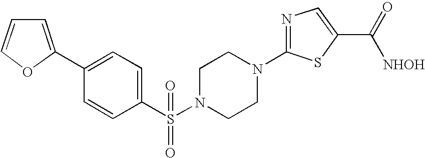

### 2-(4-{[4-(2-furyl)phenyl]sulfonyl}piperazin-1-yl)-1,3-thiazole-5-carboxylic acid hydroxyamide

Yield (17.0 mg, 9%).

¹H NMR (DMSO-d₆, 400 MHz) δ=7.96-7.65 (m, 5H, 5Ar—H), 7.23-6.68 (m, 3H, 3Ar—H), 3.6 (t, J=4.1 Hz, 4H, 2CH₂), 3.06 (t, J=4.7 Hz, 4H, 2CH₂).

LCMS=434.8 (M⁺) (M, 434.49 Calcd for C₁₈H₁₈N₄O₅S₂).

### Example 60

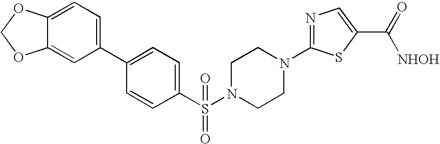

### 2-(4-{[4-(1,3-benzodioxol-5-yl)phenyl]sulfonyl}piperazin-1-yl)-1,3-thiazole-5-carboxylic acid hydroxyamide

Off-white solid.

¹H NMR (300 MHz, DMSO-d6) δ=7.87 (2H, d, J=8.8 Hz), 7.75 (2H, d, J=8.8 Hz), 7.65 (1H, bs), 7.34 (1H, d, J=2.2 Hz), 7.26 (1H, m), 7.23 (1H, d, J=2.2 Hz), 7.04 (1H, s), 7.02 (1H, s), 6.08 (2H, s), 3.52-3.60 (4H, m), 3.03-3.09 (4H, m);

LC/MS (electrospray), m/e 489 (C₂₁H₂₀N₄O₆S₂+H)

### Example 61

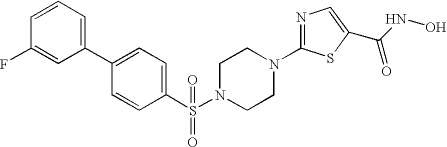

### 2-{4-[(3′-fluoro-1,1′-biphenylsulfonyl]piperazin-1-yl}-1,3-thiazole-5-carboxylic acid hydroxyamide

Off-white solid.

¹H NMR (300 MHz, DMSO-d6) δ=7.97 (2H, m), 7.81 (2H, m), 7.49-7.69 (4H, m), 7.27 (1H, m), 3.89 (2H, s), 3.11 (4H, m);

LC/MS (electrospray), m/e 463 (C₂₀H₁₉FN₄O₄S₂+H)⁺.

### Example 62

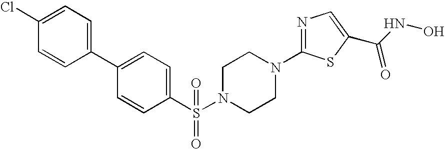

### 2-{4-[(4′-chloro-1,1′-biphenylsulfonyl]piperazin-1-yl}-1,3-thiazole-5-carboxylic acid hydroxyamide

Off-white solid.

¹H NMR (300 MHz, DMSO-d6) δ=7.94 (2H, d, J=8.4 Hz), 7.7-7.9 (4H, m), 7.56 (2H, d, J=8.4 Hz), 5.31 (1H, s), 2.28 (4H, m), 3.98 (4H, m), 2.35 (4H, m);

LC/MS (electrospray), m/e 479 (C₂₀H₁₉ClN₄O₄S₂+H)⁺.

### Example 63

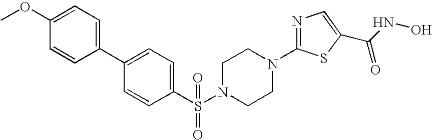

### 2-{4-[(4′-methoxy-1,1′-biphenylsulfonyl]piperazin-1-yl}-1,3-thiazole-5-carboxylic acid hydroxyamide

Off-white solid.

¹H NMR (300 MHz, DMSO-d6) δ=7.88 (2H, d, J=8.4 Hz), 7.76 (2H, d, J=8.4 Hz), 7.69 (2H, d, J=8.3 Hz), 7.65 (1H, brs), 7.05 (2H, d, J=8.3 Hz), 3.83 (3H, s), 3.57 (4H, m), 3.06 (4H, m);

LC/MS (electrospray), m/e 475 (C₂₁H₂₂N₄O₅S₂+H)⁺.

### Example 64

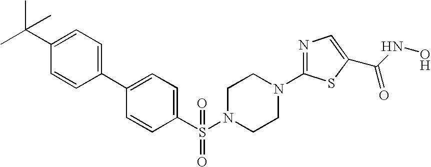

### 2-{4-[(4′-(2,2-dimethylpropyl)-1,1′-biphenylsulfonyl]piperazin-1-yl}-1,3-thiazole-5-carboxylic acid hydroxyamide

Off-white solid.

¹H NMR (300 MHz, DMSO-d6) δ=7.91 (2H, d, J=9 Hz), 7.79 (2H, d, J=9 Hz), 7.66 (2H, d, J=8.4 Hz), 7.51 (2H, d, J=8.4 Hz), 3.58 (4H, m), 3.08 (4H, m), 1.36 (9H, s);

LC/MS (electrospray), m/e 501 (C₂₄H₂₈N₄O₄S₂+H)⁺.

### Example 65

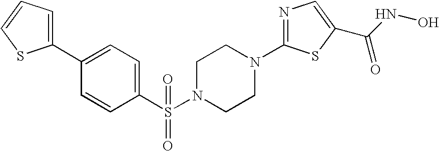

### 2-{4-[(4-thien-2-ylphenyl)sulfonyl]piperazin-1-yl}-1,3-thiazole-5-carboxylic acid hydroxamide

Off-white solid.

¹H NMR (300 MHz, DMSO-d6) δ=7.91 (2H, d, J=7.8 Hz), 7.56-7.82 (5H, m), 7.19 (1H, t, J=4.2 Hz), 3.65 (4H, m), 3.18 (4H, m);

LC/MS (electrospray), m/e 451 (C₁₈H₁₈N₄O₄S₃+H)⁺.

### Example 66

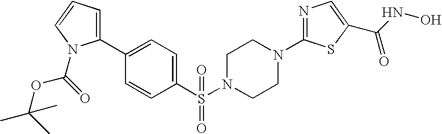

### 2-(4-{[4-(1-(2,2-dimethylprop-oxycarbonyl)-1H-pyrrol-2-yl)phenyl]-sulfonyl}-pipiperazin-1-yl)-1,3-thiazole-5-carboxylic acid hydroxyamide

Off-white solid:

¹H NMR (300 MHz, DMSO-d6) δ=7.5-7.8 (5H, m), 7.41 (1H, bs), 6.39 (1H, bs), 6.32 (1H, t, J=4.1 Hz), 3.61 (4H, m), 3.15 (4H, m), 1.24 (9H, s);

LC/MS (electrospray), m/e 534 (C₂₃H₂₇N₅O₆S₂+H)⁺.

### Example 67

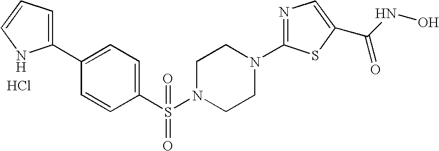

### 2-(4-{[4-(1H-pyrrol-2-yl)phenyl]sulfonyl}piperazin-1-yl)-1,3-thiazole-5-carboxylic acid hydroxyamide

Off-white solid.

¹H NMR (300 MHz, DMSO-d6) δ=7.83 (2H, d, J=8.48 Hz), 7.56-7.78 (3H, m), 6.96 (1H, s), 6.72 (1H, s), 6.17 (1H, s), 3.56 (4H, m), 3.04 (4H, m;

LC/MS (electrospray), m/e 470 (C₁₈H₂₀ClN₅O₄S₂+H)⁺.

### Example 68

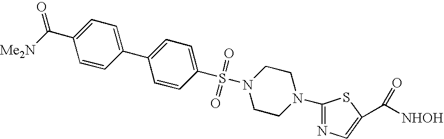

### 2-[4-(4′-N,N-dimethylcarboxamido-1,1′-biphenylsulfonyl)piperazin-1-yl]-1,3-thiazole-5-carboxylic acid hydroxyamide

A white solid (15 mg).

m/e 516 (M+H⁺).

### Example 69

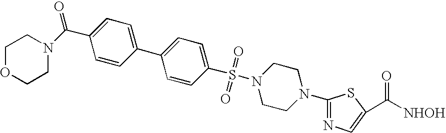

### 2-[4-(4′-(morpholin-4-ylcarbonyl)-1,1′-biphenylsulfonyl)piperazin-1-yl]-1,3-thiazole-5-carboxylic acid hydroxyamide

¹HNMR DMSO-D6 δ: 3.15 (m, 4H), 3.40-3.65 (m, 6H), 7.50 (d, 2H,), 7.62 (bs, 1H), 7.80 (dd, 4H), 8.0 (d, 2H).

### Example 70

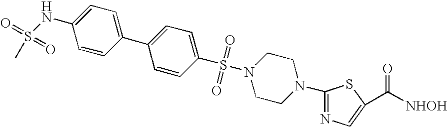

### 2-[4-(4′-methylsulfonylamino-1,1′-biphenylsulfonyl)piperazin-1-yl]-1,3-thiazole-5-carboxylic acid hydroxyamide

MS m/e: 538 (M+H⁺).

### Example 71

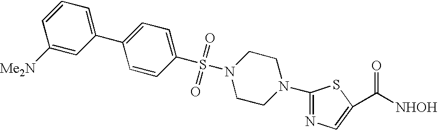

### 2-[4-(3′-(dimethylamino)-1,1′-biphenylsulfonyl)piperazin-1-yl]-1,3-thiazole-5-carboxylic acid hydroxyamide

(24 mg) M488 (M+H⁺).

### Example 72

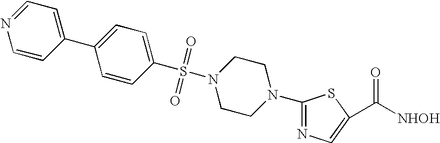

### 2-{4-[4-(pyridin-4-yl)phenylsulfonyl]piperazin-1-yl}-1,3-thiazole-5-carboxylic acid hydroxyamide

¹HNMR DMSO-D6 δ: 3.10 (m, 4H, CH2), 3.60 (bm, 4H, CH2), 7.70 (bs, 1H, olefinic), 8.0 (d, 2H, aromatic), 8.10 (d, 2H, aromatic), 8.15 (d, 2H, aromatic), 8.85 (d, 2H, aromatic), 10.0 (bs, 1H).

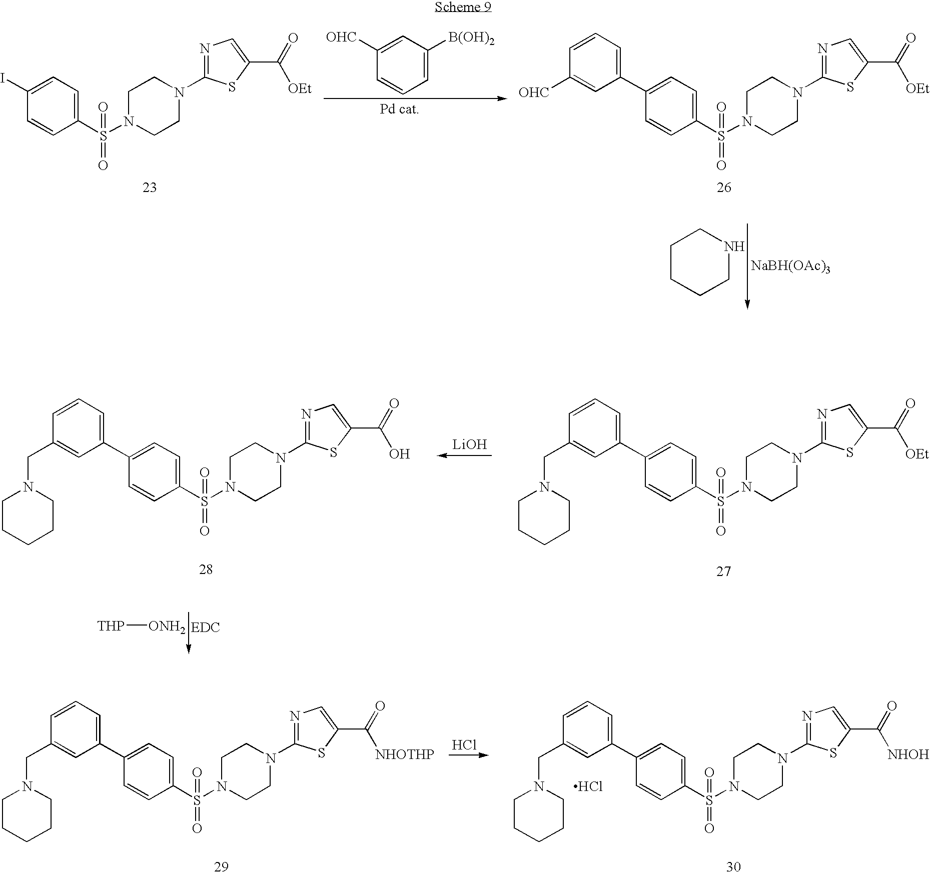

### Example 73

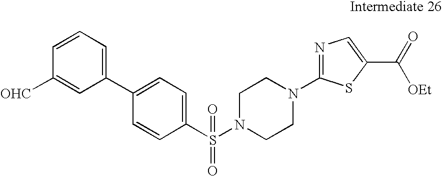

To a stirred solution of 3-formyl phenyl boronic acid (0.5 g, 0.00333 moles) in THF:water (40 mL:10 mL)) was added iodo intermediate 23 (0.85 g, 0.001675 moles) at rt. After bubbling nitrogen gas in the reaction mixture for 10.0 minutes, freshly prepared tetrakis(triphenylphosphine)palladium (0.184 g, 0.0001674 moles), CsCO₃ (4.4 g, 0.0135 moles) were added. The reaction mixture was refluxed for 8.0 h. The mixture was concentrated and the residue was partitioned between methylene chloride (100 mL) and water (50 mL). The layers were separated and the organic layer was washed with brine (25 mL), dried over anhydrous Na₂SO₄ and concentrated in vacuo. The residue was purified using silica gel column chromatography (5% MeOH in methylene chloride) to give intermediate 26 (0.6 g, 74%).

Rf=0.8.

¹H (CDCl₃, 400 MHz) δ=10.12 (s, 1H, CHO), 8.1 (s, 1H, Ar—H), 7.96 (d, J=8 Hz, 1H, Ar—H), 7.88 (d, J=8 Hz, 3H, 3Ar—H), 7.82 (d, J=8 Hz, 3H, 3Ar—H), 2.28 (q, J=8 Hz, 2H, CH2), 3.73 (t, J=4 Hz, 4H, 2CH₂), 3.22 (t, J=8 Hz, 4H, 4CH₂), 1.33 (t, J=8 Hz, 3H, CH₃).

LCMS=486 (M++1) (M, 485.571 Calcd for C₂₃H₂₃N₃O₅S).

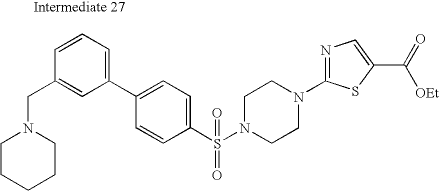

To a stirred solution of Suzuki coupled intermediate 26 (1.0 g, 0.00206 mol) in dry methylene chloride (50 mL) was added piperidine (0.21 g, 0.00246 mol) and the mixture stirred under N₂ atmosphere. After stirring for 10.0 min, sodium triacetoxy borohydride (0.521 g, 0.00246 mol) was added. The reaction was stirred overnight and treated with 10% NaHCO₃ solution (50 mL). The aqueous layer was extracted with methylene chloride (3×15 mL). The combined organic layers were washed with brine and concentrated in vacuo. The residue was purified using flash silica gel column (1% MeOH in CH₂Cl₂) to give intermediate 27 (580 mg, 85%).

Rf=0.5.

¹H NMR (CDCl₃, 400 MHz) δ=7.84-7.77 (m, 5H, 5Ar—H), 7.58 (s, 1H, Ar—H), 7.5-7.39 (m, 3H, 3Ar—H), 4.28 (q, J=8 Hz, 2H, CH₂), 3.72 (t, J=4 Hz, 4H, 2CH₂), 3.56 (s, 2H, CH₂), 3.21 (t, J=4 Hz, 4H, 2CH₂), 2.42 (bs, 4H, 2CH₂), 1.6 (m, 4H, 2CH₂), 1.46 (bs, 2H, CH₂), 1.33 (t, J=8 Hz, 3H, CH₃).

LCMS=555 (M⁺+1) (M, 554.726 Calcd for C₂₈H₃₄N₄O₄ S₂)

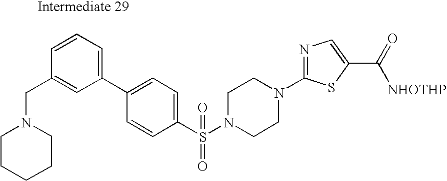

Intermediate 27 (0.53 g, 0.000955 mol) was taken in THF+MeOH (50 mL+50 mL). To the mixture was added NaOH (0.305 g, 0.0764 mol) in water (10 mL). After stirring for overnight, it was carefully neutralized with conc. HCl until a pH=7 was reached. The mixture was concentrated to dryness and crude intermediate 28 (520 mg) was taken as such for the next step.

To a solution of intermediate 28 in DCM (100 mL) was added DIEA (0.37 g, 0.002866 mol), HOBt (0.194 g, 0.00143 mol), EDC (0.366 g, 0.0019 mol) and NH₂OTHP (0.112 g, 0.000955 mol) in methylene chloride (5 mL). After stirring overnight, water (25 mL) was added and the layers were separated. The aqueous layer was extracted with methylene chloride (3×25 mL). The combined organic layers were washed with brine, dried over anhydrous sodium sulfate and concentrated in vacuo. The crude product was purified using flash silica gel column (2% MeOH in methylene chloride) to give intermediate 29 (0.35 g, 58%).

Rf=0.5.

¹H NMR (CDCl₃, 400 MHz) δ=7.84-7.77 (m, 5H, 5Ar—H), 7.60 (bs, 1H, Ar—H), 7.51-7.39 (m, 3H, 3Ar—H), 4.97 (s, 1H, CH), 3.95 (t, J=4 Hz, 4H, 2CH₂), 3.69 (s, 2H, CH₂), 3.21 (t, J=4 Hz, 4H, 4CH₂), 2.44 (bs, 4H, 2CH₂), 1.84 (bs, 4H, 2CH₂), 1.47 (bs, 8H, 4CH₂), 1.27 (bs, 2H, CH₂).

LCMS=626 (M⁺+1) (M, 625.804 Calcd for C₃₁H₃₉N₅O₅S₂).

### Example 73

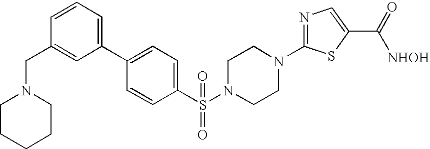

### 2-{4-[(3′-(piperidin-1-ylmethyl)-1,1′-biphenylsulfonyl]piperazin-1-yl}-1,3-thiazole-5-carboxylic acid hydroxyamide

Intermediate 29 (0.31 g, 0.495 mmol) was dissolved in dry MeOH (5 mL) and cooled to 0° C. A saturated solution of HCl in ether (10 mL) was added. After stirring for 3.0 h at 0° C., the reaction was concentrated and the residue was dried. Further purification was done using preparative HPLC to give the title compound (40 mg, 14%).

¹H NMR (DMSO-d₆, 400 MHz) δ=11.0 (bs, 1H, NH or OH), 10.44 (bs, 1H, OH or NH), 8.07-8.01 (m, 3H, 3Ar—H), 7.85 (m, 3H, 3Ar—H), 7.67-7.58 (m, 3H, 3Ar—H), 4.3 (s, 2H, CH₂), 3.65 (bs, 4H, 2CH₂), 3.3 (m, 2H, CH₂), 3.08 (bs, 4H, 2CH₂), 2.8 (m, 2H, CH₂), 1.77-1.35 (m, 6H, 3CH₂).

LCMS=542 (M⁺+1) (M, 578.188 Calcd for C₂₆H₃₂ClN₅O₄S₂).

Examples 74-90 were prepared according to Scheme 9 by the same general procedures described for example 73 and using the appropriate starting materials.

### Example 74

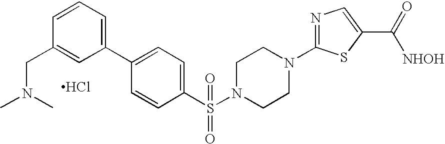

### 2-{4-[(3′-[(dimethylamino)methyl]-1,1′-biphenylsulfonyl]piperazin-1-yl}-1,3-thiazole-5-carboxylic acid hydroxyamide

¹H NMR (DMSO-d₆, 400 MHz) δ=10.92 (bs, 1H, NH or OH), 8.06-8.01 (m, 3H, 3Ar—H), 7.85 (m, 3H, Ar—H), 7.69 (bs, 1H, Ar—H), 7.63-7.58 (m, 2H, Ar—H), 4.33 (s, 2H, CH₂), 3.6 (bs, 4H, 2CH₂), 3.08 (bs, 4H, 2CH₂), 2.71 (s, 6H, N(Me)₂).

¹³C NMR (DMSO-d₆, 100 MHz) δ=144.2, 138.75, 133.93, 131.51, 131.52, 130.09, 129.78, 129.59, 129.26, 128.27, 128.00, 127.85, 127.49, 127.24, 59.25, 47.61, 44.95, 41.52.

MS m/e: 502 (M⁺+1) (M, 501.124 Calcd for C₂₃H₂₇N₅O₄S₂).

### Example 75

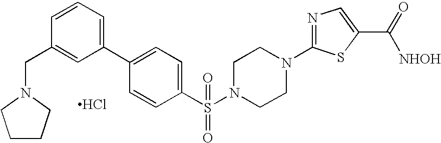

### 2-{4-[(3′-(pyrrolidin-1-ylmethyl)-1,1′-biphenylsulfonyl]piperazin-1-yl}-1,3-thiazole-5-carboxylic acid hydroxyamide

¹H NMR(DMSO-d₆, 400 MHz) d=11.09 (bs, 1H, OH or NH), 8.1 (s, 1H, Ar—H), 8.02 (d, 2H, 2Ar—H), 7.87-7.56 (m, 6H, 6Ar—H, 4.42 (s, 2H, CH2), 3.6 (bs, 4H, 2CH₂), 3.35 (m, 2H, CH₂), 3.07 (bs, 6H, 3CH₂), 2.02-1.88 (m, 4H, 2CH₂). MS m/e: 528 (M⁺+1) (M, 527.661 Calcd. for C₂₅H₂₉N₅O₄S₂).

### Example 76

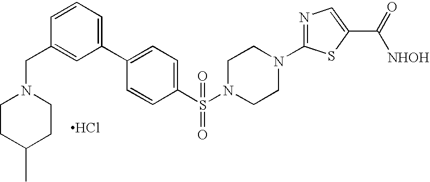

### 2-{4-[(3′-(4-methylpiperidin-1-ylmethyl)-1,1′-biphenylsulfonyl]piperazin-1-yl}-1,3-thiazole-5-carboxylic acid hydroxyamide

¹H NMR (DMSO-d₆, 400 MHz) δ=10.91 (bs, 1H, OH or NH), 9.6 (bs, 1H, OH or NH), 7.99-7.85 (m, 6H, 6Ar—H), 7.66 (m, 3H, 3Ar—H), 4.36 (s, 2H, CH₂), 3.59 (bs, 4H, 2CH₂), 3.34 (d, J=12 Hz, 2H, CH₂), 3.08 (bs, 4H, 2CH₂), 2.93 (m, 2H, CH₂), 1.77 (m, 2H, CH₂), 1.6 (m, 1H, CH), 1.33 (m, 2H, CH₂), 0.09 (d, J=8 Hz, 3H, CH₃).

MS m/e: 556 (M⁺+1) (M, 555.714 Calcd. for C₂₇H₃₃N₅O₄S₂).

### Example 77

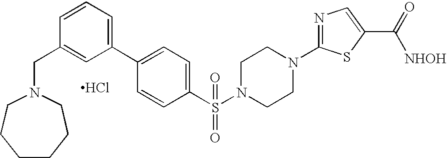

### 2-{4-[(3′-(hexahydroazepin-1-ylmethyl)-1,1′-biphenylsulfonyl]piperazin-1-yl}-1,3-thiazole-5-carboxylic acid hydroxyamide

¹H NMR (DMSO-d₆, 400 MHz) δ=11.0 (bs, 1H, OH or NH), 10.01 (bs, 1H, OH or NH), 8.01-7.59 (m, 9H, 9Ar—H), 4.4 (s, 2H, CH₂), 3.59 (bs, 4H, 2CH₂), 3.08 (bs, 4H, 2CH₂), 1.84 (m, 4H, 2CH₂), 1.62 (m, 4H, 2CH₂).

MS m/e: 556 (M⁺+1) (M, 555.714 Calcd. for C₂₇H₃₃N₅O₄S₂).

### Example 78

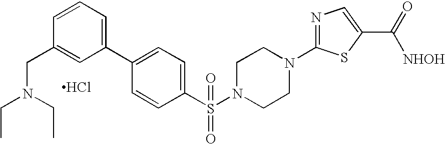

### 2-{4-[(3′-((diethylamino)methyl)-1,1′-biphenylsulfonyl]piperazin-1-yl}-1,3-thiazole-5-carboxylic acid hydroxyamide

¹H NMR (DMSO-d₆, 400 MHz) δ=10.5 (bs, 1H, NH or OH), 9.53 (bs, 1H, OH or NH), 7.99-7.59 (m, 9H, 9 Ar—H), 4.38 (s, 2H, CH₂), 3.59 (bs, 4H, 2 CH₂), 3.11 (m, 8H, 4 CH₂), 1.24 (t, J=8 Hz, 6H, 2 CH₃).

MS m/e: 530 (M⁺+1) (M, 529.677 Calcd. for C₂₅H₃₁N₅O₄S₂).

### Example 79

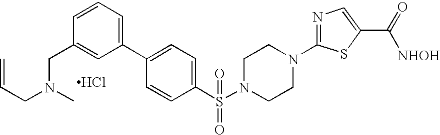

### 2-{4-[(3′-((methyl(3-propenyl)amino)methyl)phenylsulfonyl]piperazin-1-yl]-1,3-thiazole-5-carboxylic acid hydroxyamide

¹H NMR (DMSO-d₆, 400 MHz) δ=10.6 (bs, 1H, OH or NH), 10.7 (bs, 1H, OH or NH), 8.03 (m, 3H, 3Ar—H), 7.88 (m, 3H, 3Ar—H), 7.62 (m, 3H, 3Ar—H), 6.05 (m, 1H, ═CH), 5.55 (m, 2H, ═CH₂), 4.43 (m, 1H, CH), 4.27 (m, 1H, CH), 3.7 (m, 2H, CH₂), 3.5 (bs, 4H, 2CH₂), 3.08 (bs, 4H, 2CH₂), 2.6 (s, 3H, CH3).

MS m/e: 527 (M⁺+1) (M, 527.161 Calcd for C₂₅H₂₉N₅O₄S₂).

### Example 80

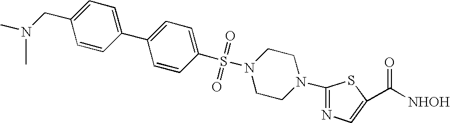

### 2-{4-[(4′-((dimethylamino)methyl)-1,1′-biphenylsulfonyl]piperazin-1-yl}-1,3-thiazole-5-carboxylic acid hydroxyamide

White solid (36 mg), 39% yield.

m/e 502 (M+H⁺).

¹HNMR DMSO-D6 δ: 2.8 (s, 6H), 3.1 (bm, 4H), 3.6 (bm, 4H), 4.3 (s, 2H) 7.55-8 (bm, 9H), 9 (bs, 1H), 10.0 (bs, 1H).

### Example 81

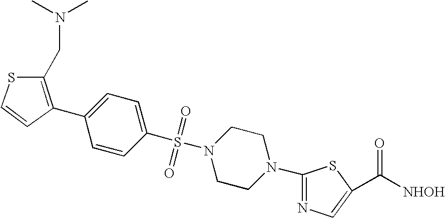

### 2-(4-{4-[(2-((dimethylamino)-methyl)thien-3-yl]phenylsulfonyl}piperazin-1-yl)-1,3-thiazole-5-carboxylic acid hydroxyamide

Gum, m/e 508 (M+H⁺).

### Example 82

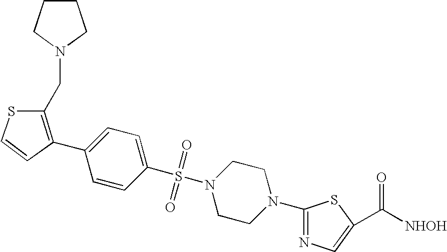

### 2-(4-[4-1 (2-(pyrrolidin-1-ylmethyl)-thien-3-yl)phenylsulfonyl}]piperazin-1-yl)-1,3-thiazole-5-carboxylic acid hydroxyamide

White solid.

¹HNMR DMSO-d₆ 8:1.88 (m, 4H), 2.9 (m, 2H), 3.08 (m, 4H), 3.35-3.8 (m, 6H), 4.63 (m, 2H), 7.24 (d, 1H), 7.69 (m, 3H), 7.83 (m, 3H), 9.85 (bs, 1H),

m/e 535 (M+H⁺).

### Example 83

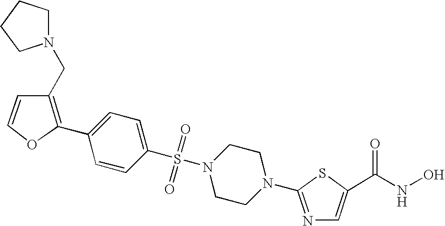

### 2-(4-{4-[(3-(pyrrolidin-1-ylmethyl)-2-furyl]phenylsulfonly}piperazin-1-yl)-1,3-thiazole-5-carboxylic acid hydroxyamide

White solid.

H¹NMR DMSO-d₆ δ: 1.85 (m, 2H), 1.99 (m, 2H), 3.08 (m, 8H), 3.58 (m, 4H), 4.52 (d, 2H), 6.86 (d, 1H), 7.74 (m, 1H), 7.8-7.92 (m, 5H), 9.85 (bs, 1H),

m/e 535 (M+H⁺).

### Example 84

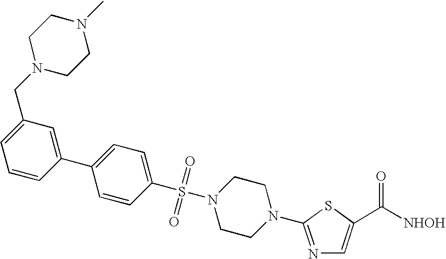

### 2-{4-[(3′-((4-methylpiperazin-1-yl)methyl 1,1′-biphenylsulfonyl]-piperazin-1-yl}-1,3-thiazole-5-carboxylic acid hydroxyamide

Floculant white solid (6 mg),

m/e 557 (M+H⁺)

¹HNMR DMSO-d₆ δ: 2.78 (m, 3H), 3.0-4 (m, 18H), 7.43 (d, 1H), 7.52 (t, 1H), 7.65 (bs, 1H), 7.72 (d, 2H), 7.83 (d, 2H), 7.93 (d, 2H).

### Example 85

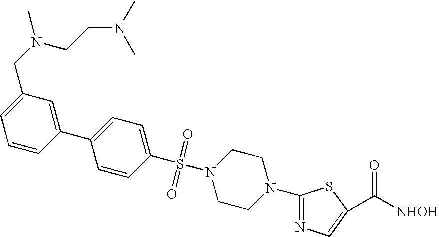

### 2-{4-[3′-{[(2-(dimethylamino)ethyl)(methyl)amino]methyl}-1,1′-biphenylsulfonyl]piperazin-1-yl}-1,3-thiazole-5-carboxylic acid hydroxyamide

Floculant white solid (6 mg),

m/e 557 (M+H⁺).

¹HNMR DMSO-d₆ δ: 2.81 (m, 9H), 3.0-4 (m, 14H), 7.6-7.82 (m, 5H), 7.85 (d, 2H), 7.95 (d, 2H).

### Example 86

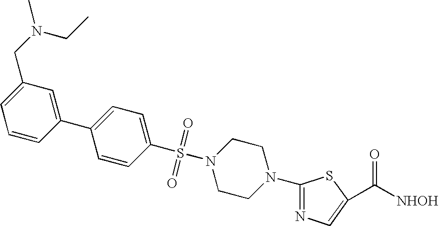

### 2-{4-[(3′-[ethyl(methyl)amino]methyl-1,1′-biphenylsulfonyl]piperazin-1-yl)-1,3-thiazole-5-carboxylic acid hydroxyamide

Floculant white solid (7 mg), m/e 516 (M+H⁺).

### Example 87

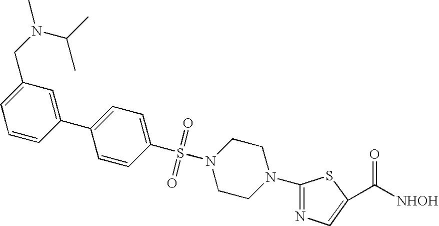

### 2-{4-[(3′-[isopropyl(methyl)amino]methyl]-1,1′-biphenylsulfonyl]-piperazin-1-yl}-1,3-thiazole-5-carboxylic acid hydroxyamide

Floculant white solid (6 mg), m/e 530 (M+H⁺).

### Example 88

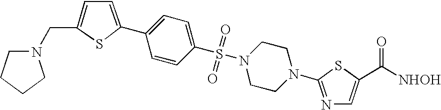

### 2-(4-{4-[(5-(pyrrolidin-1-ylmethyl)-thien-2-yl]phenylsulfonyl}piperazin-1-yl)-1,3-thiazole-5-carboxylic acid hydroxyamide

Floculant white solid (3 mg),

m/e 535 (M+H⁺),

¹HNMR DMSO-d₆ δ:1.88 (m, 2H), 2.05 (m, 2H), 3.1 (m, 8H), 3.6 (m, 4H), 4.65 (m, 2H), 7.38 (m, 1H), 7.65 (m, 2H), 7.78 (d, 2H), 7.9 (d, 2H), 9.95 (bs, 1H), 10.85 (bs, 1H),

m/e 535 (M+H⁺).

### Example 89

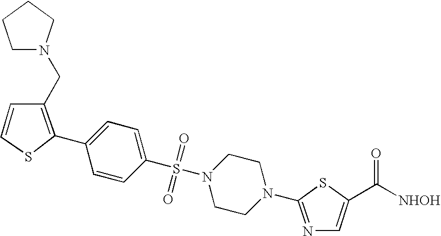

### 2-(4-{4-[(3-(pyrrolidin-1-ylmethyl)-thien-2-yl]phenylsulfonyl}piperazin-1-yl)-1,3-thiazole-5-carboxylic acid hydroxyamide

Floculant white solid (8 mg),

m/e 535 (M+H⁺).

¹HNMR DMSO-d₆ δ:1.80 (m, 4H), 3.85 (m, 2H), 3.1 (m, 4H), 3.4 (m, 2H), 3.65 (m, 4H), 4.45 (d, 2H), 7.38 (d, 1H), 7.66 (bm, 1H), 7.73 (d, 2H), 7.81-7.88 (m, 3H), 7.9 (d, 2H), 9.95 (bs, 1H), 10.85 (bs, 1H), m/e 535 (M+H⁺).

### Example 90

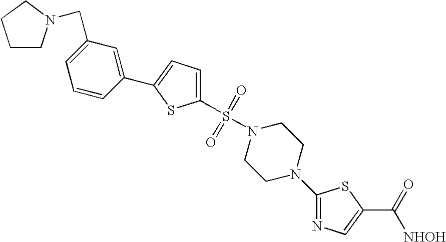

### 2-(4-{5-[3-(pyrrolidin-1-ylmethyl)phenyl]thiophene-2-sulfonyl}piperazin-1-yl)-1,3-thiazole-5-carboxylic acid hydroxyamide

Floculant white solid (3.5 mg),

m/e 534 (M+H⁺).

¹HNMR DMSO-d₆ δ:1.85 (m, 2H), 2.08 (m, 2H), 3.15 (m, 8H), 3.68 (m, 4H), 4.4 (m, 2H), 7.5-7.9 (m, 2H), 7.66 (bm, 1H), 7.71 (m, 2H), 7.86 (m, 1H) 7.91 (s, 1H), 9.95 (bs, 1H), 10.9 (bs, 1H), m/e 534 (M+H⁺).

### BIOLOGICAL EXAMPLES

### Example A

### In Vitro Fluorescent Histone Deacetylase Assay

Histone deacetylase (HDAC) activity assays were performed using the HDAC fluorescent activity assay/drug discovery kit (Biomol Research Laboratories, Plymouth Meeting, Pa.) essentially according to the manufacturer's instructions. The included HeLa cell nuclear extract, which contains a mosaic of HDAC enzymes and other nuclear factors, was used as the source of HDAC activity. The final substrate concentration in the assay mixture was 50 μM. The reaction was allowed to proceed for 10 min at room temperature before stopping the reaction. Test compounds were prepared as 20 mM stock solutions in DMSO (Molecular Biology grade, Sigma-Aldrich Co. St. Louis, Mo.) and stored at −70° C. Serial dilutions of test compounds were prepared in assay buffer immediately prior to testing. DMSO was determined in a separate trial to have no significant effect on the activity of this assay at concentrations up to 5%; the final DMSO concentration in the wells was no more than 2% and therefore DMSO effects were safely neglected. Assays were performed in white polystyrene 96-well half-area assay plates (Corning, Corning, N.Y.) and measured on a Wallace 1420 fluorescent plate reader (Wallac Oy, Turku, Finland) with an excitation wavelength of 355 nm, an emission wavelength of 460 nm, and a 1 sec signal averaging time.

The following table shows the percent inhibition of HDAC produced by some of the examples of the present invention at a concentration of 100 μM.

| Example | % inhibition HDAC | Example | % inhibition HDAC |
|---|---|---|---|
| Number | @ 100 μM | Number | @ 100 μM |
| 1 | 94 | 38 | 100 |
| 3 | 93 | 39 | 98 |
| 4 | 100 | 95 | 98 |
| 9 | 97 | 46 | 98 |
| 5 | 85 | 106 | 99 |
| 2 | 99 | 105 | 99 |
| 10 | 88 | 43 | 99 |
| 7 | 98 | 94 | 98 |
| 11 | 98 | 42 | 98 |
| 99 | 99 | 44 | 98 |
| | | 51 | 100 |
| 6 | 97 | 47 | 96 |
| 8 | 98 | 45 | 95 |
| 13 | 99 | 82 | 99 |
| 12 | 98 | 88 | 99 |
| | | 84 | 100 |
| 48 | 96 | 85 | 100 |
| 15 | 92 | 100 | 96 |
| 18 | 95 | 52 | 96 |
| 17 | 96 | 89 | 99 |
| 20 | 97 | 87 | 99 |
| 16 | 100 | 86 | 99 |
| 49 | 85 | 61 | 95 |
| 14 | 98 | 60 | 99 |
| 19 | 99 | 96 | 100 |
| 21 | 100 | 98 | 98 |
| 22 | 100 | 91 | 99 |
| 74 | 100 | 53 | 94 |
| 23 | 99 | 54 | 100 |
| 24 | 100 | 92 | 100 |
| 25 | 98 | 93 | 100 |
| 26 | 98 | 97 | 99 |
| 68 | 99 | | |
| 70 | 99 | 73 | 100 |
| 80 | 99 | 29 | 100 |
| 27 | 99 | 63 | 98 |
| 28 | 100 | | |
| 71 | 100 | | |
| 75 | 100 | | |
| 50 | 99 | | |
| 101 | 100 | 64 | 48 |
| 102 | 99 | 65 | 99 |
| 69 | 100 | 66 | 99 |
| 81 | 100 | 67 | 100 |
| 103 | 100 | 55 | 9 |
| 104 | 100 | 76 | 99 |
| 56 | 98 | 77 | 100 |
| 32 | 99 | 78 | 100 |
| 30 | 99 | 79 | 99 |
| 31 | 99 | 62 | 98 |
| 72 | 98 | 99 | 99 |
| 35 | 97 | | |
| 34 | 92 | | |
| 33 | 96 | | |
| 37 | 99 | | |
| 57 | 93 | | |
| 58 | 95 | 83 | 100 |
| 59 | 99 | 90 | 99 |
| 36 | 98 | | |
| 41 | 98 | | |
| 40 | 100 | | |

In some assays recombinant HDAC8 (Biomol) was used as the source of the enzyme activity; here the final substrate concentration was 250 μM, the final concentration of HDAC8 was 0.02 Units/μL and the reaction was allowed to proceed at 37° C. for 1 h before stopping. For all curves, IC₅₀ values were calculated with the GraFit curve-fitting program (Erithacus, Horley, Surrey,

### Example B

### Whole Cell Cytotoxicity Assay:Sulforhodamine B

The following procedure can be found on the Developmental Therapeutics Program NCI/NIH web site at http://dtp.nci.nih.gov/brancehes/btb/ivclsp.html.

1. Human tumor cell lines of HT29, A549 and MCF7 are grown in DMEM containing 10% fetal bovine serum and 2 mM L-glutamine. Cells are plated in a 96 well plate at a density of 5000 cells per well in 100 μL of growth medium and incubated at 37° C., 5% CO₂, for 24 hours prior to the addition of experimental compounds.

2. Experimental drugs are solubilized in DMSO for a final concentration of 20 mM immediately prior to use. Drugs are further diluted in growth media for a total of nine drug concentrations and a growth control. At the 24 hour time point, one plate of cells is fixed in situ with TCA as a measurement of the cell population at time zero, or the time of drug addition.

3. The plates are further incubated with drug for an additional 48 hours.

4. The cells are fixed in situ by gently aspirating off the culture media and then adding 50 μL of ice cold 10% TCA per well and incubated at 4° C. for 60 minutes. The plates are washed with tap water five times and allowed to air dry for 5 minute.

5. 50 μL of a 0.4% (w/v) Sulforhodamine B solution in 1% (v/v) acetic acid is added per well and incubated for 30 minutes at room temperature.

6. Following staining, plates are washed five times with 1% acetic acid to remove any unbound dye and then allowed to air dry for 5 minutes.

7. Stain is solubilized with 100 μL of 10 mM Tris pH 10.5 per well and placed on an orbital rotator for 5 minutes.

8. Absorbance is read at 570 nm.

The following table shows the percent inhibition of MCF7 cell growth produced by some of the examples of the present invention at a concentration of 100 μM.

| | % inhibition | | % inhibition |
|---|---|---|---|
| Example | MCF7 cells | Example | MCF7 cells |
| Number | @ 100 μM | Number | @ 100 μM |
| 1 | 95 | 38 | 97.8 |
| 3 | 95.3 | 39 | 97.9 |
| 4 | 95.4 | 95 | 96.8 |
| 9 | 97.7 | 46 | 77.3 |
| 5 | 97.9 | 106 | 82.3 |
| 2 | 98.3 | 105 | 93 |
| 10 | 97.8 | 43 | 91 |
| 7 | 83.3 | 94 | 90.6 |
| 11 | 96.7 | 42 | 90 |
| | | 44 | 91.7 |
| | | 51 | 97 |
| 6 | 95.1 | 47 | 90.2 |
| 8 | 90.1 | 45 | 78.7 |
| 13 | 98.9 | 82 | 92.7 |
| 12 | 95.5 | 88 | 93.8 |
| | | 84 | 96.7 |
| 48 | 89.4 | 85 | 97.5 |
| 15 | 88.5 | 100 | 96.8 |
| 18 | 96.6 | 52 | 97.3 |
| 17 | 97.8 | 89 | 98.3 |
| 20 | 97.9 | 87 | 97.7 |
| 16 | 90.9 | 86 | 97.8 |
| 49 | 95.4 | 61 | 92.2 |
| 14 | 89.8 | 60 | 92.1 |
| 19 | 76.5 | 96 | 85.6 |
| 21 | 97.1 | 98 | 92.2 |
| 22 | 98 | 91 | 94.6 |
| 74 | 97.3 | 53 | 89.9 |
| 23 | 96.7 | 54 | 97.1 |
| 24 | 95.3 | 92 | 88.8 |
| 25 | 90.1 | 93 | 92.7 |
| 26 | 90 | 97 | 83.9 |
| 68 | 84.7 | | |
| 70 | 83.6 | 73 | 96.7 |
| 80 | 97.9 | 29 | 98.5 |
| 27 | 88.5 | 63 | 93.3 |
| 28 | 92.7 | | |
| 71 | 97.2 | | |
| 75 | 98.6 | | |
| 50 | 98.3 | | |
| 101 | 98.1 | 64 | 87 |
| 102 | 84 | 65 | 95 |
| 69 | 87.7 | 66 | 97 |
| 81 | 97.1 | 67 | 98 |
| 103 | 97.5 | 55 | 0 |
| 104 | 97.6 | 76 | 98 |
| 56 | 96.9 | 77 | 98 |
| 32 | 98.1 | 78 | 99 |
| 30 | 97.9 | 79 | 98 |
| 31 | 97.7 | 62 | 75 |
| 72 | 84.5 | 99 | 96 |
| 35 | 97.9 | | |
| 34 | 97.7 | | |
| 33 | 98.4 | | |
| 37 | 98 | | |
| 57 | 68.6 | | |
| 58 | 97.6 | 83 | 98 |
| 59 | 98.9 | 90 | 98 |
| 36 | 98.2 | | |
| 41 | 98.1 | | |
| 40 | 99.1 | | |

1. A compound of formula I: 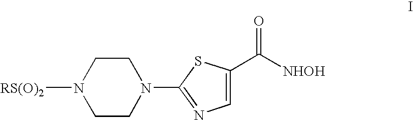
wherein:
R is selected from the group consisting of alkyl, substituted alkyl, alkenyl, substituted alkenyl, amino, substituted amino, aryl, substituted aryl, cycloalkyl, substituted cycloalkyl, heleroaryl, substituted heteroaryl, heterocyclic and substituted heterocyclic;
or tautomers, isomers, prodrugs and pharmaceutically acceptable salts thereof
with the proviso that R is not 4-aminophenyl.

2. The compound of claim 1 wherein R is aryl.

3. The compound of claim 2 wherein R is phenyl or naphthyl.

4. The compound of claim 1 wherein R is substituted aryl.

5. The compound of claim 4 wherein R is substituted phenyl.

6. The compound of claim 5 wherein R is selected from the group consisting of:
3-methoxyphenyl, 4-methoxyphenyl, 3,4-dimethoxyphenyl, 4-trifluoro-methoxyphenyl, 2-trifluroromethylphenyl, 3-trifluroromethylphenyl, 4-tri-fluoromethylphenyl, 4-nitrophenyl, 4-acetylphenyl, 4-[(N-morpholino)-methyl]phenyl, 4-[(N-pyrrolidinyl)methyl]phenyl, 4-(N,N-dimethylamino-methyl)phenyl, 5-(N,N-dimethylamino)naphthyl, 4-pyrrolind-1-ylphenyl, 4-acetamidophenyl, 4-methyl-2,3-dihydrobenzisoxazinyl, 2,3-dihydro-benzofuran-5-yl, 2,1,3-benzothiadiazol-5-yl, 2,3-dihydro-1,4-benzodioxin-6-yl, 2-chlorophenyl, 2-chloro-6-methylphenyl, 3-chloro-2,5-dimethylphenyl, 3-chlorophenyl, 3,4-dichlorophenyl, 3-chloro-2-methylphenyl, 3-chloro-4-methylphenyl, 4-chlorophenyl, 3-cyanophenyl, 2-fluorophenyl, 3-fluorophenyl, 3,4-difluorophenyl, 3,5-difluorophenyl, 4-fluorophenyl, 4-fluoro-2-methylphenyl, 3-fluoro-4-methylphenyl, 5-fluoro-2-methylphenyl, 4-methyl-sulfonylphenyl, 2-methylphenyl, 3-methylphenyl, 3-hydroxymethylphenyl, 3-(N,N-dimethylaminomethyl)phenyl, 3-(pyrrolidin-1-yl)methylphenyl, 4-ethylphenyl, 4-methylphenyl, 3,4-dimethylphenyl, 3,5-dimethylphenyl, 4-iso-propylphenyl, 4-n-propylphenyl, t-butylphenyl, (4-pyrazol-1-yl)phenyl, 3-biphenyl, 4-biphenyl, 4-(3-N,N-dimethylaminomethylphenyl)phenyl, 4-(3-N,N-dimethylaminophenyl)phenyl, 4[(3-pyrrolind-N-ylmethyl)-phenyl]phenyl, 4[(N-morpholinocarbonyl)phenyl]phenyl, 4-(N,N-dimethyl-aminocarbonylphenyl)phenyl, 4-(4-fluorophenyl)phenyl, (pyrid-4-yl)phenyl, 4-(3-chlorophenyl)phenyl, 4-(2-chlorophenyl)phenyl, 4-(3-fluorophenyl)-phenyl, 4-(2-furanyl)phenyl, 4 [2-(pyrrrolidin-N-ylmethyl)thien-3-yl]phenyl, 4-[5-(pyrrrolidin-N-ylmethyl)thien-2-yl]phenyl, 4-[3-(pyrrrolidin-N-ylmethyl)thien-2-yl]phenyl, 4-[3-(4-methylpiperidin-1-yl)phenyl]phenyl, 4-[(3-(N-methyl-N-{2-N,N-dimethyleth-1-yl}aminomethyl)phenyl]phenyl, 4-[(3-(N-methyl-N-ethylaminomethyl)phenyl]phenyl, 4[(3-(N-methyl-N-isopropyl}aminomethyl)phenyl]phenyl, 4-(methylsulfonamidophenyl)phenyl, 4-[1,3-(benzodioxol-5-yl)]phenyl, 4-(pyrimidin-5-yl)phenyl, 2-(2-methyl-thiopyrimidin-4-yl)thien-5-yl, 4-[4-(acetamidophenyl)]phenyl, 4-(2-N,N-dimethylaminothien-3-yl)phenyl, and 4-(pyrid-3-yl)phenyl.

7. The compound according to claim 1 wherein R is heteroaryl.

8. The compound according to claim 7 wherein R is selected from the group consisting of thien-2yl, pyrid-2-yl, pyrid-3-yl, and benzothio-furan-2-yl.

9. The compound according to claim 1 wherein R is substituted heteroaryl.

10. The compound according to claim 9 wherein R is substituted heteroaryl.

11. The compound according to claim 10 wherein R is selected from the group consisuing of 3,5-dimethylisoxazol-4-yl, 2-(4-morpholino)-pyrid-5-yl, and 2-phenoxypyrid-5-yl.

12. The compound according to claim 1 wherein R is alkyl or substituted alkyl.

13. The compound according to claim 12 wherein R is selected from the group consisting n-butyl, benzyl, and 2-phenylethyl.

14. The compound according to claim 1 wherein R is alkenyl or substituted alkenyl.

15. The compound according to claim 14 wherein R is trans-2-phenylethen-1-yl.

16. The compound according to claim 1 wherein R is amino or substituted amino.

17. The compound according to claim 16 wherein R is dimethylamino.

18. The compound according to claim 1 wherein R is a heterocyclic group.

19. The compound according to claim 18 wherein R is 1-methyl-imidazol-4-yl.

20. A compound of formula II: 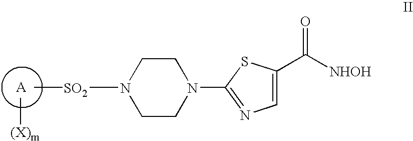
wherein:
A is selected from the group consisting of aryl and heteroaryl;
X is selected from the group consisting of acyl, acylamino, alkyl, substituted alkyl, alkoxy, substituted alkoxy, amino, substituted amino, aminoacyl, aryloxy, substituted aryloxy, cyano, halo, heterocyclic, substituted heterocyclic, nitro, thioalkyl, substituted thioalkyl, and R²—S(O)₂(NH)ₙ— where R² is selected from the group consisting of alkyl, substituted alkyl, aryl, substituted aryl, heteroaryl and substituted heteroaryl;
m is zero, one, two or three; and
n is zero or one;
or tautomers, isomers, prodrugs and pharmaceutically acceptable salts thereof
with the proviso that -A-(X)ₘ is not 4-aminophenyl.

21. The compound according to claim 20 wherein the substituent defined by 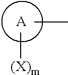
is selected from the group consisting of phenyl, naphthyl, 3-methoxyphenyl, 4-methoxyphenyl, 3,4-dimethoxyphenyl, 4-trifluoromethoxyphenyl, 2-trifluroromethylphenyl, 3-trifluroromethylphenyl, 4-trifluroromethylphenyl, 4-nitrophenyl, 4-acetylphenyl, 4-[(N-morpholino)methyl]phenyl, 4-[(N-pyrrolidinyl)methyl]phenyl, 4-(N,N-dimethylaminomethyl)phenyl, 5-(N,N-dimethylamino)naphthyl, 4-pyrrolind-1-ylphenyl, 4-acetamidophenyl, 4-methyl-2,3-dihydrobenzisoxazinyl, 2,3-dihydrobenzofuran-5-yl, 2,1,3-benzothiadiazol-5-yl, 2,3-dihydro-1,4-benzodioxin-6-yl, 2-chlorophenyl, 2-chloro-6-methylphenyl, 3-chloro-2,5-dimethylphenyl, 3-chlorophenyl, 3,4-dichlorophenyl, 3-chloro-2-methylphenyl, 3-chloro-4-methylphenyl, 4-chlorophenyl, 3-cyanophenyl, 2-fluorophenyl, 3-fluorophenyl, 3,4-difluorophenyl, 3,5-difluorophenyl, 4-fluorophenyl, 4-fluoro-2-methylphenyl, 3-fluoro-4-methylphenyl, 5-fluoro-2-methylphenyl, 4-methylsulfonylphenyl, 2-methylphenyl, 3-methylphenyl, 3-hydroxymethyl-phenyl, 3-(N,N-dimethylaminomethyl)phenyl, 3-(pyrrolidin-1-yl)methylphenyl, 4-ethylphenyl, 4-methylphenyl, 3,4-dimethylphenyl, 3,5-dimethylphenyl, 4-iso-propylphenyl, 4-n-propylphenyl, t-butylphenyl, thien-2-yl, pyrid-2-yl, pyrid-3-yl, benzothiofuran-2-yl, 3,5-dimethylisoxazol-4-yl, 2-(4-morpholino)pyrid-5-yl, and 2-phenoxypyrid-5-yl.

22. A compound of formula III: 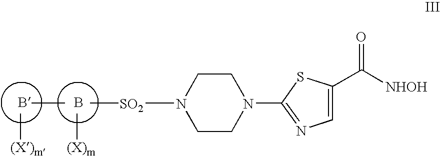
wherein:
B and B′ are independently selected from the group consisting of aryl and heteroaryl;
X and X′ are independently selected from the group consisting of acyl, acylamino, alkyl, substituted alkyl, alkoxy, substituted alkoxy, amino, substituted amino, aminoacyl, aryloxy, substituted aryloxy, cyano, halo, heteroaryl, substituted heteroaryl, heterocyclic, substituted heterocyclic, nitro, thioalkyl, substituted thioalkyl, R²—S(O)₂(NH)ₙ—, where R² is selected from the group consisting of alkyl, substituted alkyl, aryl, substituted aryl, heteroaryl and substituted heteroaryl, and
m′ is zero, one, two or three;
m′ is zero, one or two; and
n is zero or one
or tautomers, isomers, prodrugs and pharmaceutically acceptable salts thereof.

23. The compound according to claim 22 wherein the substituent defined by the formula: 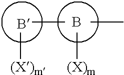
is selected from the group consisting of (4-pyrazol-1-yl)phenyl, 3-biphenyl, 4-bi-phenyl, 4-(3-N,N-dimethylaminomethylphenyl)phenyl, 4-(3-N,N-dimethyl-aminophenyl)phenyl, 4-[(3-pyrrolind-N-ylmethyl)phenyl]phenyl, 4-[(N-morpholinocarbonyl)phenyl]phenyl, 4-(N,N-dimethylaminocarbonyl-phenyl)phenyl, 4-(4-fluorophenyl)phenyl, 4-(pyrid-4-yl)phenyl, 4-(3-chloro-phenyl)phenyl, 4-(2-chlorophenyl)phenyl, 4-(3-fluorophenyl)phenyl, 4-(2-furanyl)phenyl, 4-[2-(pyrrrolidin-N-ylmethyl)thien-3-yl]phenyl, 4-[5-(pyrrrolidin-N-ylmethyl)thien-2-yl]phenyl, 4-[3-(pyrrrolidin-N-ylmethyl)thien-2-yl]phenyl, 4-[3-(4-methylpiperidin-1-yl)phenyl]phenyl, 4[(3-(N-methyl-N-{2-N,N-dimethyleth-1-yl}aminomethyl)phenyl]phenyl, 4[(3-(N-methyl-N-ethylaminomethyl)phenyl]phenyl, 4 [(3-(N-methyl-N-isopropyl}aminomethyl)phenyl]phenyl, 4-(methylsulfonamidophenyl)phenyl, 4-[1,3-(benzodioxol-5-yl)]phenyl, 4-(pyrimidin-5-yl)phenyl, 2-(2-methylthiopyrimidin-4-yl)thien-5-yl, 4-[4-(acetamidophenyl)]phenyl, 4-(2-N,N-dimethylaminothien-3-yl)phenyl, and 4-(pyrid-3-yl)phenyl.

24. A compound selected from the group consisting of:
2-[4-(naphtha-2-yl-4-sulfonyl)-piperazin-1-yl]-thiazole-5-carboxylic acid hydroxyamide
2-[4-(4-trifluoromethoxy-benzene sulfonyl)-piperazin-1-yl]-thiazole-5-carboxylic acid hydroxyamide
2-[4-(4-toluene-4-sulfonyl)-piperazin-1-yl]-thiazole-5-carboxylic acid hydroxyamide
2-[4-(biphenyl-4-sulfonyl)-piperazin-1-yl]-thiazole-5-carboxylic acid hydroxyamide
2-[4-(4-trifluoromethyl-benzenesulfonyl)-piperazin-1-yl]-thiazole-5-carboxylic acid hydroxyamide
2-[4-(3,4-dimethoxy-benzene sulfonyl)-piperazin-1-yl]-thiazole-5-carboxylic acid hydroxyamide
2-[4-(5-dimethylamino-naphthalene-1-sulfonyl)-piperazin-1-yl]-thiazole-5-carboxylic acid hydroxyamide
2-[4-(4-acetyl-benzenesulfonyl)-piperazin-1-yl]-thiazole-5-carboxylic acid hydroxyamide
2-[4-(benzenesulfonyl)-piperazin-1-yl]-thiazole-5-carboxylic acid hydroxyamide
2-[4-(4-nitro-benzenesulfonyl)-piperazin-1-yl]-thiazole-5-carboxylic acid hydroxyamide
2-[4-(4-fluoro-benzenesulfonyl)-piperazin-1-yl]-thiazole-5-carboxylic acid hydroxyamide
2-[4-(4-pyrolidinylbenzenesulfonyl)-piperazin-1-yl]-thiazole-5-carboxylic acid hydroxyamide
2-[4-(thiophene-2-benzenesulfonyl)-piperazin-1-yl]-thiazole-5-carboxylic acid hydroxyamide
2-[4-(N-methyl-2,3-dihydrobenzisoxazinylsulfonyl)-piperazin-1-yl]-thiazole-5-carboxylic acid hydroxyamide
2-[4-(4-isopropylphenylsulfonyl)-piperazin-1-yl]-thiazole-5-carboxylic acid hydroxyamide
2-[4-(trans-2-phenylethanelsulfonyl)-piperazin-1-yl]-thiazole-5-carboxylic acid hydroxyamide
2-[4-(2-chlorophenylsulfonyl)-piperazin-1-yl]-thiazole-5-carboxylic acid hydroxyamide
2-[4-(3-chlorophenylsulfonyl)-piperazin-1-yl]-thiazole-5-carboxylic acid hydroxyamide
2-[4-(3,4-dichlorophenylsulfonyl)piperazin-1-yl]-thiazole-5-carboxylic acid hydroxyamide
2-[4-(N,N-dimethylsulfonyl)-piperazin-1-yl]-thiazole-5-carboxylic acid hydroxyamide
2-[4-(4-methylsufonylphenylsulfonyl)-piperazin-1-yl]-thiazole-5-carboxylic acid hydroxyamide
2-[4-(pyridine-3-sulfonyl)-piperazin-1-yl]-thiazole-5-carboxylic acid hydroxyamide
2-[4-(2-methylphenylsulfonyl)-piperazin-1-yl]-thiazole-5-carboxylic acid hydroxyamide
2-[4-(3-methylphenylsulfonyl)-piperazin-1-yl]-thiazole-5-carboxylic acid hydroxyamide
2-{4-[(3′-[(dimethylamino)methyl]-1,1′-biphenylsulfonyl]piperazin-1-yl}-1,3-thiazole-5-carboxylic acid hydroxyamide
2-[4-(4-n-propylphenylsulfonyl)-piperazin-1-yl]-thiazole-5-carboxylic acid hydroxyamide
2-[4-(3,5-dimethylphenylsulfonyl)-piperazin-1-yl]-thiazole-5-carboxylic acid hydroxyamide
2-[4-(4-t-butylphenylsulfonyl)-piperazin-1-yl]-thiazole-5-carboxylic acid hydroxyamide
2-[4-(benzylsulfonyl)-piperazin-1-yl]-thiazole-5-carboxylic acid hydroxyamide
2-[4-(4′-N,N-dimethylcarboxamido-1,1′-biphenylsulfonyl)piperazin-1-yl]-1,3-thiazole-5-carboxylic acid hydroxyamide
2-[4-(4′-methylsulfonylamino-1,1′-biphenylsulfonyl)piperazin-1-yl]-1,3-thiazole-5-carboxylic acid hydroxyamide
2-{4-[(4′-((dimethylamino)methyl}1,1′-biphenylsulfonyl]piperazin-1-yl}-1,3-thiazole-5-carboxylic acid hydroxyamide
2-[4-(3,5-dimethylisoxazolesulfonyl)piperazin-1-yl]-thiazole-5-carboxylic acid hydroxyamide
2-[4-({4-morpholino}-3-pyridylsulfonyl)-piperazin-1-yl]-thiazole-5-carboxylic acid hydroxyamide
2-[4-(3′-(dimethylamino)-1,1′-biphenylsulfonyl)piperazin-1-yl]-1,3-thiazole-5-carboxylic acid hydroxyamide
2-{4-[(3′-(pyrrolidin-1-ylmethyl 1,1′-biphenylsulfonyl]piperazin-1-yl}-1,3-thiazole-5-carboxylic acid hydroxyamide
2-[4-(4-dimethylaminophenylsulfonyl)-piperazin-1-yl]-thiazole-5-carboxylic acid hydroxyamide
2-[4-(3,4-dimethylphenylsulfonyl)-piperazin-1-yl]-thiazole-5-carboxylic acid hydroxyamide
2-[4-(4′-(morpholin-4-ylcarbonyl)-1,1′-biphenylsulfonyl)piperazin-1-yl]-1,3-thiazole-5-carboxylic acid hydroxyamide
2-(4-{4-[(2-((dimethylamino)-methyl)thien-3-yl]phenylsulfonyl}piperazin-1-yl)-1,3-thiazole-5-carboxylic acid hydroxyamide
2-{4-[(4′-fluoro-1,1′-biphenyl-4-yl)sulfonyl]piperazin-1-yl}-1,3-thiazole-5-carboxylic acid hydroxyamide
2-[4-(3-chloro-2-methylphenylsulfonyl)-piperazin-1-yl]-thiazole-5-carboxylic acid hydroxyamide
2-[4-(3-chloro-4-methylphenylsulfonyl)-piperazin-1-yl]-thiazole-5-carboxylic acid hydroxyamide
2-[4-(4-methoxyphenylsulfonyl)-piperazin-1-yl]-thiazole-5-carboxylic acid hydroxyamide
2-{4-[4-(pyridin-4-yl)phenylsulfonyl]piperazin-1-yl}-1,3-thiazole-5-carboxylic acid hydroxyamide
2-[4-(3-fluorophenylsulfonyl)-piperazin-1-yl]-thiazole-5-carboxylic acid hydroxyamide
2-[4-(2-fluorophenylsulfonyl)-piperazin-1-yl]-thiazole-5-carboxylic acid hydroxyamide
2-[4-(2-methyl-5-fluorophenylsulfonyl)-piperazin-1-yl]-thiazole-5-carboxylic acid hydroxyamide
2-[4-(2-trifluoromethylphenylsulfonyl)-piperazin-1-yl]-thiazole-5-carboxylic acid hydroxyamide
2-{4-[(3′-chloro-1,1′-biphenyl-4-yl)sulfonyl]piperazin-1-yl}-1,3-thiazole-5-carboxylic acid hydroxyamide
2-{4-[(2′-chloro-1,1′-biphenyl-4-yl)sulfonyl]piperazin-1-yl}-1,3-thiazole-5-carboxylic acid hydroxyamide
2-(4-{[4-(2-furyl)phenyl]sulfonyl}piperazin-1-yl)-1,3-thiazole-5-carboxylic acid hydroxyamide
2-[4-(3,4-difluorophenylsulfonyl)-piperazin-1-yl]-thiazole-5-carboxylic acid hydroxyamide
2-[4-(4-fluoro-2-methylphenylsulfonyl)-piperazin-1-yl]-thiazole-5-carboxylic acid hydroxyamide
2-[4-(3-fluoro-4-methylphenylsulfonyl)-piperazin-1-yl]-thiazole-5-carboxylic acid hydroxyamide
2-[4-(2-methyl-6-chlorophenylsulfonyl)piperazin-1-yl]-thiazole-5-carboxylic acid hydroxyamide
2-[4-(2,5-dimethyl-4-chlorophenylsulfonyl)-piperazin-1-yl]-thiazole-5-carboxylic acid hydroxyamide
2-[4-(2,1,3-benzothiadiazole-5-sulfonyl)-piperazin-1-yl]-thiazole-5-carboxylic acid hydroxyamide
2-[4-(2-benzothiphenesulfonyl)-piperazin-1-yl]-thiazole-5-carboxylic acid hydroxyamide
2-[4-(2,3-dihydrobenzofuransulfonyl)-piperazin-1-yl]-thiazole-5-carboxylic acid hydroxyamide
2-[4-(3,4-benzodioxansulfonyl)-piperazin-1-yl]-thiazole-5-carboxylic acid hydroxyamide
2-[4-(3-biphenylsulfonyl)piperazin-1-yl]-thiazole-5-carboxylic acid hydroxyamide
2-[4-(2-phenoxypyridine-5-sulfonyl)-piperazin-1-yl]-thiazole-5-carboxylic acid hydroxyamide
2-[4-(2-{2-methylthiopyrimidine-4-yl}-5-thiophenesulfonyl)-piperazin-1-yl]-thiazole-5-carboxylic acid hydroxyamide
2-(4-[4-{(2-(pyrrolidin-1-ylmethyl)-thien-3-yl)phenylsulfonyl}]-piperazin-1-yl)-1,3-thiazole-5-carboxylic acid hydroxyamide
2-(4-{4-[(5-(pyrrolidin-1-ylmethyl)-thien-2-yl]phenylsulfonyl}-piperazin-1-yl)-1,3-thiazole-5-carboxylic acid hydroxyamide
2-{4-[(3′-((4-methylpiperazin-1-yl)methyl)-1,1′-biphenylsulfonyl]-piperazin-1-yl}-1,3-thiazole-5-carboxylic acid hydroxyamide
2-{4-[3′-{[(2-(dimethylamino)ethyl)(methyl)amino]methyl}-1,1′-biphenylsulfonyl]piperazin-1-yl}-1,3-thiazole-5-carboxylic acid hydroxyamide
2-[4-(3,5-difluorophenylsulfonyl)-piperazin-1-yl]-thiazole-5-carboxylic acid hydroxyamide
2-(4-{4-[(3-(pyrrolidin-1-ylmethyl)-thien-2-yl]phenylsulfonyl}-piperazin-1-yl)-1,3-thiazole-5-carboxylic acid hydroxyamide
2-{4-[(3′-[isopropyl(methyl)amino]methyl]-1,1′-biphenylsulfonyl]-piperazin-1-yl}-1,3-thiazole-5-carboxylic acid hydroxyamide
2-{4-[(3′-[ethyl(methyl)amino]methyl]-1,1′-biphenylsulfonyl]-piperazin-1-yl}-1,3-thiazole-5-carboxylic acid hydroxyamide
2-{4-[(3′-fluoro-1,1′-biphenylsulfonyl]piperazin-1-yl}-1,3-thiazole-5-carboxylic acid hydroxyamide
2-(4-{[4-(1,3-benzodioxol-5-yl)phenyl]sulfonyl}piperazin-1-yl)-1,3-thiazole-5-carboxylic acid hydroxyamide
2-[4-(n-butylsulfonyl)-piperazin-1-yl]-thiazole-5-carboxylic acid hydroxyamide
2-[4-(chlorophenylsulfonyl)-piperazin-1-yl]-thiazole-5-carboxylic acid hydroxyamide
2-{4-[(5-bromothien-2-yl)sulfonyl]piperazin-1-yl}-N-hydroxy-1,3-thiazole-5-carboxamide
2-{4-[(4′-chloro-1,1′-biphenylsulfonyl]piperazin-1-yl}-1,3-thiazole-5-carboxylic acid hydroxyamide
2-{4-[(4′-methoxy-1,1′-biphenylsulfonyl]piperazin-1-yl}-1,3-thiazole-5-carboxylic acid hydroxyamide
2-{4-[(4′-(2,2-dimethylpropyl)-1,1′-biphenylsulfonyl]piperazin-1-yl}-1,3-thiazole-5-carboxylic acid hydroxyamide
2-{4-[(4-thien-2-ylphenyl)sulfonyl]piperazin-1-yl}-1,3-thiazole-5-carboxylic acid hydroxamide
2-(4-{[4-(1-(2,2-dimethylprop-oxycarbonyl 1H-pyrrol-2-yl)phenyl]-sulfonyl}-piperazin-1-yl)-1,3-thiazole-5-carboxylic acid hydroxyamide
2-(4-{[4-(1H-pyrrol-2-yl)phenyl]sulfonyl}piperazin-1-yl)-1,3-thiazole-5-carboxylic acid hydroxyamide
2-{4-[(3′-(piperidin-1-ylmethyl)-1,1′-biphenylsulfonyl]piperazin-1-yl}-1,3-thiazole-5-carboxylic acid hydroxyamide
2-{4-[(3′-(4-methylpiperidin-1-ylmethyl)-1,1′-biphenylsulfonyl]-piperazin-1-yl}-1,3-thiazole-5-carboxylic acid hydroxyamide
2-{4-[(3′-(hexahydroazepin-1-ylmethyl)-1,1′-biphenylsulfonyl]-piperazin-1-yl}-1,3-thiazole-5-carboxylic acid hydroxyamide
2-{4-[(3′-((diethylamino)methyl}1,1′-biphenylsulfonyl]piperazin-1-yl}-1,3-thiazole-5-carboxylic acid hydroxyamide
2-{4-[(3′-((methyl(3-propenyl)amino)methyl)phenylsulfonyl]piperazin-1-yl}-1,3-thiazole-5-carboxylic acid hydroxyamide
2-(4-{4-[(3-(pyrrolidin-1-ylmethyl)-2-furyl]phenylsulfonyl}piperazin-1-yl)-1,3-thiazole-5-carboxylic acid hydroxyamide
2-(4-{5-[3-(pyrrolidin-1-ylmethyl)phenyl]thiophene-2-sulfonyl}piperazin-1-yl)-1,3-thiazole-5-carboxylic acid hydroxyamide
2-[4-(4-pyrzaol-1-yl-4-sulfonyl)-piperazin-1-yl]-thiazole-5-carboxylic acid hydroxyamide
2-[4-(4-{1-methylimidazol-4-yl}-4-sulfonyl)-piperazin-1-yl]-thiazole-5-carboxylic acid hydroxyamide
2-[4-(4-methoxyphenyl-4-sulfonyl)-piperazin-1-yl]-thiazole-5-carboxylic acid hydroxyamide
2-[4-{3-trifluoromethylphenyl}-4-sulfonyl)-piperazin-1-yl]-thiazole-5-carboxylic acid hydroxyamide
2-[4-(4-{N,N-dimethylaminomethylphenyl}-4-sulfonyl)-piperazin-1-yl]-thiazole-5-carboxylic acid hydroxyamide
2-[4-(4-{N-morpholinomethylphenyl}-4-sulfonyl)-piperazin-1-yl]-thiazole-5-carboxylic acid hydroxyamide
2-[4-(4-{N-pyrrolidinylmethylphenyl}-4-sulfonyl)-piperazin-1-yl]-thiazole-5-carboxylic acid hydroxyamide
2-[4-(4-phenethylphenyl-4-sulfonyl)-piperazin-1-yl]-thiazole-5-carboxylic acid hydroxyamide
2-[4-(4-ethylphenyl-4-sulfonyl)-piperazin-1-yl]-thiazole-5-carboxylic acid hydroxyamide
2-[4-(3-hydroxymethylphenyl-4-sulfonyl)-piperazin-1-yl]-thiazole-5-carboxylic acid hydroxyamide
2-[4-(3-pyrrolidin-1-ylmethylphenyl-4-sulfonyl)-piperazin-1-yl]-thiazole-5-carboxylic acid hydroxyamide
2-[4-(4-pyrimid-5-ylphenyl-4-sulfonyl)-piperazin-1-yl]-thiazole-5-carboxylic acid hydroxyamide
2-{4-[(4′-(acetamidophenyl)-phenylsulfonyl]piperazin-1-yl}-1,3-thiazole-5-carboxylic acid hydroxyamide
2-[4-(3-pyridylphenyl-4-sulfonyl)-piperazin-1-yl]-thiazole-5-carboxylic acid hydroxyamide
2-[4-(3-{N,N-dimethylaminomethylphenyl}-4-sulfonyl)-piperazin-1-yl]-thiazole-5-carboxylic acid hydroxyamide
2-[4-(3-methoxymethylbenzenesulfonyl)-piperazin-1-yl]-thiazole-5-carboxylic acid hydroxyamide
2-[4-(4-acetamido-4-sulfonyl)-piperazin-1-yl]-thiazole-5-carboxylic acid hydroxyamide
2-[4-(3-cyanophenyl}-4-sulfonyl)-piperazin-1-yl]-thiazole-5-carboxylic acid hydroxyamide
or tautomers, isomers, prodrugs and pharmaceutically acceptable salts thereof.

25. A pharmaceutical composition comprising an effective amount of one or more compounds according to any of claims 1, 20, 22 or 24 and a pharmaceutically inert carrier.

26. A pharmaceutical composition comprising an effective amount of one or more compounds according to any of claims 1, 20, 22 or 24 and an effective amount of at least one anti-cancer agent, and a pharmaceutically inert carrier.

27. The pharmaceutical composition of claim 26, wherein the anti-cancer agent is selected from platinum coordination compounds, taxane compounds, topoisomerase I inhibitors, topoisomerase II inhibitors, anti-tumour vinca alkaloids, anti-tumour nucleoside derivatives, alkylating agents, anti-tumour anthracycline derivatives, HER2 antibodies, estrogen receptor antagonists, selective estrogen receptor modulators, aromatase inhibitors, retinoids, retinoic acid metabolism blocking agents (RAMBA), DNA methyl transferase inhibitors, kinase inhibitors, farnesyltransferase inhibitors, and other HDAC inhibitors.

28. The pharmaceutical composition of claim 27, wherein the anti-cancer agent is selected from carboplatin, oxalyplatin, paclitaxel, docetaxel, irinotecan, topotecan, etoposide, teniposide, vinblastine, vincristine, vinorelbine, 5-fluorouracil, gemcitabine, capecitabine, cyclophosphamide, chlorambucil, carmustine, lomustine, daunorubicin, doxorubicin, darubicin, mitoxantrone, trastuzuma, tamoxifen, toremifene, droloxifene, faslodex, raloxifene, exemestane, anastrozole, letrazole, vorozole, vitamin D, accutane, azacytidine, flavoperidol, imatinib mesylate, and gefitinib.

29. A method for inhibiting a proliferative disorder in a mammalian patient which method comprises administering to said patient a pharmaceutical composition according to claim 25.

30. A method for inhibiting a proliferative disorder in a mammalian patient which method comprises administering to said patient a pharmaceutical composition according to claim 26.

31. The method of claim 30, wherein the at least one other anti-cancer agent is selected from platinum coordination compounds, taxane compounds, topoisomerase I inhibitors, topoisomerase II inhibitors, anti-tumour vinca alkaloids, anti-tumour nucleoside derivatives, alkylating agents, anti-tumour anthracycline derivatives, HER2 antibodies, estrogen receptor antagonists, selective estrogen receptor modulators, aromatase inhibitors, retinoids, retinoic acid metabolism blocking agents (RAMBA), DNA methyl transferase inhibitors, kinase inhibitors, farnesyltransferase inhibitors, and other HDAC inhibitors.

32. The method of claim 31, wherein the at least one other anti-cancer agent is selected from carboplatin, oxalyplatin, paclitaxel, docetaxel, irinotecan, topotecan, etoposide, teniposide, vinblastine, vincristine, vinorelbine, 5-fluorouracil, gemcitabine, capecitabine, cyclophosphamide, chlorambucil, carmustine, lomustine, daunorubicin, doxorubicin, darubicin, mitoxantrone, trastuzuma, tamoxifen, toremifene, droloxifene, faslodex, raloxifene, exemestane, anastrozole, letrazole, vorozole, vitamin D, accutane, azacytidine, flavoperidol, imatinib mesylate, and gefitinib.
